SEQUENCE LISTING (135 sequences)

SEQ ID NO: 1 - RNA, 21 nt, Homo sapiens
aaauggugcccuagugacuac

SEQ ID NO: 2 - RNA, 24 nt, Homo sapiens
aauccuuggaaccuaggugugagu

SEQ ID NO: 3 - RNA, 65 nt, Homo sapiens
cuugaauccuuggaaccuaggugugagugcuauuucagugcaacacaccuauucaaggauucaaa

SEQ ID NO: 4 - RNA, 22 nt, Homo sapiens
accacugaccguugacuguacc

SEQ ID NO: 5 - RNA, 22 nt, Homo sapiens
ugagguaguagguuguauaguu

SEQ ID NO: 6 - RNA, 80 nt, Homo sapiens
ugggaugagguaguagguuguauaguuuuagggucacacccaccacugggagauaacuauacaaucuacugucuuuccua

SEQ ID NO: 7 - RNA, 22 nt, Homo sapiens
ugagguaguagguuguaugguu

SEQ ID NO: 8 - RNA, 84 nt, Homo sapiens
gcauccggguugagguaguagguuguaugguuuagaguuacacccugggaguuaacuguacaaccuucuagcuuuccuuggagc

SEQ ID NO: 9 - RNA, 22 nt, Homo sapiens
agagguaguagguugcauaguu

SEQ ID NO: 10 - RNA, 87 nt, Homo sapiens
ccuaggaagagguaguagguugcauaguuuuagggcagggauuuugcccacaaggagguaacuauacgaccugcugccuuucuuagg

SEQ ID NO: 11 - RNA, 22 nt, Homo sapiens
ugagguaggagguuguauaguu

SEQ ID NO: 12 - RNA, 79 nt, Homo sapiens
cccgggcugagguaggagguuguauaguugaggaggacacccaaggagaucacuauacggccuccuagcuuuccccagg

SEQ ID NO: 13 - RNA, 22 nt, Homo sapiens
ugagguaguaguuuguacaguu

SEQ ID NO: 14 - RNA, 84 nt, Homo sapiens
aggcugagguaguaguuuguacaguuugagggucuaugauaccacccgguacaggagauaacuguacaggccacugccuugcca

SEQ ID NO: 15 - RNA, 22 nt, Homo sapiens
ugagguaguaguuugugcuguu

SEQ ID NO: 16 - RNA, 84 nt, Homo sapiens
cuggcugagguaguaguuugugcuguuggucggguugugacauugcccgcuguggagauaacugcgcaagcuacugccuugcua

SEQ ID NO: 17 - RNA, 21 nt, Homo sapiens
uaaagugcugacagugcagau

SEQ ID NO: 18 - RNA, 82 nt, Homo sapiens
ccugccggggcuaaagugcugacagugcagauagugguccucuccgugcuaccgcacuguggguacuugcugcuccagcagg

SEQ ID NO: 19 - RNA, 22 nt, Homo sapiens
uucacauugugcuacugucugc

SEQ ID NO: 20 - RNA, 89 nt, Homo sapiens
ugcugcuggccagagcucuuuucacauugugcuacugucugcaccugucacuagcagugcaauguuaaaagggcauuggccguguagug

SEQ ID NO: 21 - RNA, 22 nt, Homo sapiens
cagugcaaugaugaaagggcau

SEQ ID NO: 22 - RNA, 82 nt, Homo sapiens
ggccugcccgacacucuuucccuguugcacuacuauaggccgcugggaagcagugcaaugaugaaagggcaucggucagguc

SEQ ID NO: 23 - RNA, 22 nt, Homo sapiens
uaacagucuacagccauggucg

SEQ ID NO: 24 - RNA, 101 nt, Homo sapiens
ccgcccccgcgucuccagggcaaccguggcuuucgauuguuacugugggaacuggagguaacagucuacagccauggucgccccgcagcacgcccacgcgc

SEQ ID NO: 25 - RNA, 23 nt, Homo sapiens
uguaguguuuccuacuuuaugga

SEQ ID NO: 26 - RNA, 87 nt, Homo sapiens
gacagugcagucacccauaaaguagaaagcacuacuaacagcacuggaggguguaguguuuccuacuuuauggaugaguguacugug

SEQ ID NO: 27 - RNA, 21 nt, Homo sapiens
ugagaugaagcacuguagcuc

SEQ ID NO: 28 - RNA, 106 nt, Homo sapiens
gcgcagcgcccugucucccagccugaggugcagugcugcaucucuggucaguugggagucugagaugaagcacuguagcucaggaagagagaaguuguucugcagc

SEQ ID NO: 29 - RNA, 23 nt, Homo sapiens
guccaguuuucccaggaaucccu

SEQ ID NO: 30 - RNA, 88 nt, Homo sapiens
caccuuguccucacgguccaguuuucccaggaaucccuuagaugcuaagauggggauuccuggaaauacuguucuugaggucaugguu

SEQ ID NO: 31 - RNA, 22 nt, Homo sapiens
ucagugcacuacagaacuuugu

SEQ ID NO: 32 - RNA, 68 nt, Homo sapiens
gaggcaaaguucugagacacuccgacucugaguaugauagaagucagugcacuacagaacuuugucuc

SEQ ID NO: 33 - RNA, 22 nt, Homo sapiens
ucagugcaucacagaacuuugu

SEQ ID NO: 34 - RNA, 99 nt, Homo sapiens
caagcacgauuagcauuugaggugaaguucuguuauacacucaggcuguggcucucugaaagucagugcaucacagaacuuugucucgaaagcuuucua

SEQ ID NO: 35 - RNA, 21 nt, Homo sapiens
ucgaggagcucacagucuagu

SEQ ID NO: 36 - RNA, 90 nt, Homo sapiens
uuuccugcccucgaggagcucacagucuaguaugucucauccccuacuagacugaagcuccuugaggacagggauggucauacucaccuc

SEQ ID NO: 37 - RNA, 22 nt, Homo sapiens
uagcagcacaucaugguuuaca

SEQ ID NO: 38 - RNA, 98 nt, Homo sapiens
uugaggccuuaaaguacuguagcagcacaucaugguuuacaugcuacagucaagaugcgaaucauuauuugcugcucuagaaauuuaaggaaauucau

SEQ ID NO: 39 - RNA, 22 nt, Homo sapiens
uagcagcacguaaauauuggcg

SEQ ID NO: 40 - RNA, 88 nt, Homo sapiens
gucagcagugccuuagcagcacguaaauauuggcguuaagauucuaaaauuaucuccaguauuaacugugcugcugaaguaagguuga

SEQ ID NO: 41 - RNA, 22 nt, Homo sapiens
aacauucauugcugucgguggg

SEQ ID NO: 42 - RNA, 110 nt, Homo sapiens
ccugugcagagauuauuuuuuaaaaggucacaaucaacauucauugcugucgguggguugaacuguguggacaagcucacugaacaaugaaugcaacuguggccccgcuu

SEQ ID NO: 43 - RNA, 21 nt, Homo sapiens
cugaccuaugaauugacagcc

SEQ ID NO: 44 - RNA, 110 nt, Homo sapiens
gccgagaccgagugcacagggcucugaccuaugaauugacagccagugcucucgucuccccucuggcugccaauuccauaggucacagguauguucgccucaaugccagc

SEQ ID NO: 45 - RNA, 22 nt, Homo sapiens
aacuggcccucaaagucccgcu

SEQ ID NO: 46 - RNA, 83 nt, Homo sapiens
guggucucagaaucgggguuuugagggcgagaugaguuuauguuuuauccaacuggcccucaaagucccgcuuuuggggucau

SEQ ID NO: 47 - RNA, 22 nt, Homo sapiens
uguaacagcaacuccaugugga

SEQ ID NO: 48 - RNA, 85 nt, Homo sapiens
augguguuaucaaguguaacagcaacuccauguggacuguguaccaauuuccaguggagaugcuguuacuuuugaugguuaccaa

SEQ ID NO: 49 - RNA, 22 nt, Homo sapiens
uguaacagcaacuccaugugga

SEQ ID NO: 50 - RNA, 85 nt, Homo sapiens; identical to SEQ ID NO: 48

SEQ ID NO: 51 - RNA, 22 nt, Homo sapiens
uagguaguuuccuguuguuggg

SEQ ID NO: 52 - RNA, 84 nt, Homo sapiens
acuggucggugauuuagguaguuuccuguuguugggauccaccuuucucucgacagcacgacacugccuucauuacuucaguug

SEQ ID NO: 53 - RNA, 22 nt, Homo sapiens
uucaccaccuucuccacccagc

SEQ ID NO: 54 - RNA, 75 nt, Homo sapiens
ggcugugccggguagagagggcagugggagguaagagcucuucacccuucaccaccuucuccacccagcauggcc

SEQ ID NO: 55 - RNA, 23 nt, Homo sapiens
cccaguguucagacuaccuguuc

SEQ ID NO: 56 - RNA, 71 nt, Homo sapiens
gccaacccaguguucagacuaccuguucaggaggcucucaauguguacaguagucugcacauugguuaggc

SEQ ID NO: 57 - RNA, 23 nt, Homo sapiens
ugugcaaaucuaugcaaaacuga

SEQ ID NO: 58 - RNA, 82 nt, Homo sapiens
gcaguccucuguuaguuuugcauaguugcacuacaagaagaauguaguugugcaaaucuaugcaaaacugaugguggccugc

SEQ ID NO: 59 - RNA, 23 nt, Homo sapiens
ugugcaaauccaugcaaaacuga

SEQ ID NO: 60 - RNA, 87 nt, Homo sapiens
cacuguucuaugguuaguuuugcagguuugcauccagcugugugauauucugcugugcaaauccaugcaaaacugacugugguagug

SEQ ID NO: 61 - RNA, 22 nt, Homo sapiens
uaacacugucugguaacgaugu

SEQ ID NO: 62 - RNA, 90 nt, Homo sapiens
ccgggccccugugagcaucuuaccggacagugcuggauuucccagcuugacucuaacacugucugguaacgauguucaaaggugacccgc

SEQ ID NO: 63 - RNA, 22 nt, Homo sapiens
uaauacugccugguaaugauga

SEQ ID NO: 64 - RNA, 95 nt, Homo sapiens
ccagcucgggcagccguggccaucuuacugggcagcauuggauggagucaggucucuaauacugccugguaaugaugacggcggagcccugcacg

SEQ ID NO: 65 - RNA, 22 nt, Homo sapiens
gugaaauguuuaggaccacuag

SEQ ID NO: 66 - RNA, 110 nt, Homo sapiens
guguuggggacucgcgcgcuggguccagugguucuuaacaguucaacaguucuguagcgcaauugugaaauguuuaggaccacuagacccggcgggcgcggcgacagcga

SEQ ID NO: 67 - RNA, 23 nt, Homo sapiens
uaaagugcuuauagugcagguag

SEQ ID NO: 68 - RNA, 71 nt, Homo sapiens
guagcacuaaagugcuuauagugcagguaguguuuaguuaucuacugcauuaugagcacuuaaaguacugc

SEQ ID NO: 69 - RNA, 22 nt, Homo sapiens
cugugcgugugacagcggcuga

SEQ ID NO: 70 - RNA, 110 nt, Homo sapiens
acccggcagugccuccaggcgcagggcagccccugcccaccgcacacugcgcugccccagacccacugugcgugugacagcggcugaucugugccugggcagcgcgaccc

SEQ ID NO: 71 - RNA, 22 nt, Homo sapiens
acagcaggcacagacaggcagu

SEQ ID NO: 72 - RNA, 110 nt, Homo sapiens
ggccuggcuggacagaguugucaugugucugccugucuacacuugcugugcagaacauccgcucaccuguacagcaggcacagacaggcagucacaugacaacccagccu

SEQ ID NO: 73 - RNA, 22 nt, Homo sapiens
aagcugccaguugaagaacugu

SEQ ID NO: 74 - RNA, 85 nt, Homo sapiens
ggcugagccgcaguaguucuucaguggcaagcuuuauguccugacccagcuaaagcugccaguugaagaacuguugcccucugcc

SEQ ID NO: 75 - RNA, 21 nt, Homo sapiens
caagucacuagugguuccguu

SEQ ID NO: 76 - RNA, 81 nt, Homo sapiens
gggcuuucaagucacuagugguuccguuuaguagaugauugugcauuguuucaaaauggugcccuagugacuacaaagccc

SEQ ID NO: 77 - RNA, 22 nt, Homo sapiens
cauugcacuugucucggucuga

SEQ ID NO: 78 - RNA, 84 nt, Homo sapiens
ggccaguguugagaggcggagacuugggcaauugcuggacgcugcccugggcauugcacuugucucggucugacagugccggcc

SEQ ID NO: 79 - RNA, 22 nt, Homo sapiens
uucaaguaauccaggauaggcu

SEQ ID NO: 80 - RNA, 77 nt, Homo sapiens
guggccucguucaaguaauccaggauaggcugugcaggucccaaugggccuauucuugguuacuugcacggggacgc

SEQ ID NO: 81 - RNA, 21 nt, Homo sapiens
uucacaguggcuaaguuccgc

SEQ ID NO: 82 - RNA, 78 nt, Homo sapiens
cugaggagcagggcuuagcugcuugugagcaggguccacaccaagucguguucacaguggcuaaguuccgccccccag

SEQ ID NO: 83 - RNA, 21 nt, Homo sapiens
uucacaguggcuaaguucugc

SEQ ID NO: 84 - RNA, 97 nt, Homo sapiens
accucucuaacaaggugcagagcuuagcugauuggugaacagugauugguuuccgcuuuguucacaguggcuaaguucugcaccugaagagaaggug

SEQ ID NO: 85 - RNA, 22 nt, Homo sapiens
uagcaccaucugaaaucgguua

SEQ ID NO: 86 - RNA, 64 nt, Homo sapiens
augacugauuucuuuugguguucagagucaauauaauuuucuagcaccaucugaaaucgguuau

SEQ ID NO: 87 - RNA, 22 nt, Homo sapiens
uguaaacauccucgacuggaag

SEQ ID NO: 88 - RNA, 71 nt, Homo sapiens
gcgacuguaaacauccucgacuggaagcugugaagccacagaugggcuuucagucggauguuugcagcugc

SEQ ID NO: 89 - RNA, 22 nt, Homo sapiens
uguaaacauccuacacucagcu

SEQ ID NO: 90 - RNA, 88 nt, Homo sapiens
accaaguuucaguucauguaaacauccuacacucagcuguaauacauggauuggcugggagguggauguuuacuucagcugacuugga

SEQ ID NO: 91 - RNA, 23 nt, Homo sapiens
uguaaacauccuacacucucagc

SEQ ID NO: 92 - RNA, 89 nt, Homo sapiens
accaugcuguaguguguguaaacauccuacacucucagcugugagcucaagguggcugggagaggguuguuuacuccuucugccaugga

SEQ ID NO: 93 - RNA, 22 nt, Homo sapiens
uguaaacauccccgacuggaag

SEQ ID NO: 94 - RNA, 70 nt, Homo sapiens
guuguuguaaacauccccgacuggaagcuguaagacacagcuaagcuuucagucagauguuugcugcuac

SEQ ID NO: 95 - RNA, 22 nt, Homo sapiens
uguaaacauccuugacuggaag

SEQ ID NO: 96 - RNA, 92 nt, Homo sapiens
gggcagucuuugcuacuguaaacauccuugacuggaagcuguaagguguucagaggagcuuucagucggauguuuacagcggcaggcugcca

SEQ ID NO: 97 - RNA, 21 nt, Homo sapiens
aggcaagaugcuggcauagcu

SEQ ID NO: 98 - RNA, 71 nt, Homo sapiens
ggagaggaggcaagaugcuggcauagcuguugaacugggaaccugcuaugccaacauauugccaucuuucc

SEQ ID NO: 99 - RNA, 22 nt, Homo sapiens
uggcagugucuuagcugguugu

SEQ ID NO: 100 - RNA, 110 nt, Homo sapiens
ggccagcugugaguguuucuuuggcagugucuuagcugguuguugugagcaauaguaaggaagcaaucagcaaguauacugcccuagaagugcugcacguuguggggccc

SEQ ID NO: 101 - RNA, 22 nt, Homo sapiens
uaaugccccuaaaaauccuuau

SEQ ID NO: 102 - RNA, 87 nt, Homo sapiens
accgcagggaaaaugagggacuuuugggggcagauguguuuccauuccacuaucauaaugccccuaaaaauccuuauugcucuugca

SEQ ID NO: 103 - RNA, 21 nt, Homo sapiens
ugguagacuauggaacguagg

SEQ ID NO: 104 - RNA, 67 nt, Homo sapiens
agagaugguagacuauggaacguaggcguuaugauuucugaccuauguaacaugguccacuaacucu

SEQ ID NO: 105 - RNA, 22 nt, Homo sapiens
acuggacuuagggucagaaggc

SEQ ID NO: 106 - RNA, 90 nt, Homo sapiens
gagagaagcacuggacuuagggucagaaggccugagucucucugcugcagaugggcucucugucccugagccaagcuuuguccucccugg

SEQ ID NO: 107 - RNA, 21 nt, Homo sapiens
acuggacuuggagucagaagg

SEQ ID NO: 108 - RNA, 66 nt, Homo sapiens
agggcuccugacuccagguccuguguguuaccuagaaauagcacuggacuuggagucagaaggccu

SEQ ID NO: 109 - RNA, 22 nt, Homo sapiens
cagcagcaauucauguuuugaa

SEQ ID NO: 110 - RNA, 98 nt, Homo sapiens
cgaggggauacagcagcaauucauguuuugaaguguucuaaaugguucaaaacgugaggcgcugcuauacccccucguggggaagguagaaggugggg

SEQ ID NO: 111 - RNA, 22 nt, Homo sapiens
aguggggaacccuuccaugagg

SEQ ID NO: 112 - RNA, 84 nt, Homo sapiens
uugacuuagcuggguaguggggaacccuuccaugaggaguagaacacuccuuaugcaagauucccuucuaccuggcuggguugg

SEQ ID NO: 113 - RNA, 22 nt, Homo sapiens
aaacaaacauggugcacuucuu

SEQ ID NO: 114 - RNA, 82 nt, Homo sapiens
ugguaccugaaaagaaguugcccauguuauuuucgcuuuauaugugacgaaacaaacauggugcacuucuuuuucgguauca

SEQ ID NO: 115 - RNA, 23 nt, Homo sapiens
uagcagcgggaacaguucugcag

SEQ ID NO: 116 - RNA, 71 nt, Homo sapiens
ugcccuagcagcgggaacaguucugcagugagcgaucggugcucugggguauuguuuccgcugccagggua

SEQ ID NO: 117 - RNA, 22 nt, Homo sapiens
uauugcacuugucccggccugu

SEQ ID NO: 118 - RNA, 78 nt, Homo sapiens
cuuucuacacagguugggaucgguugcaaugcuguguuucuguaugguauugcacuugucccggccuguugaguuugg

SEQ ID NO: 119 - RNA, 23 nt, Homo sapiens
uuuggcacuagcacauuuuugcu

SEQ ID NO: 120 - RNA, 78 nt, Homo sapiens
uggccgauuuuggcacuagcacauuuuugcuugugucucuccgcucugagcaaucaugugcagugccaauaugggaaa

SEQ ID NO: 121 - RNA, 23 nt, Mus musculus
uuaaugcuaauugugauaggggu

SEQ ID NO: 122 - RNA, 65 nt, Mus musculus
cuguuaaugcuaauugugauagggguuuuggccucugacugacuccuaccuguuagcauuaacag

SEQ ID NO: 123 - RNA, 21 nt, Mus musculus
cugaccuaugaauugacagcc

SEQ ID NO: 124 - RNA, 89 nt, Mus musculus
cgugcacagggcucugaccuaugaauugacagccaguacucuuuucucuccucuggcugccaauuccauaggucacagguauguucacc

SEQ ID NO: 125 - RNA, 22 nt, Mus musculus
acaguagucugcacauugguua

SEQ ID NO: 126 - RNA, 110 nt, Mus musculus
ccagaggauaccuccacuccgucuacccaguguuuagacuaccuguucaggacucccaaauuguacaguagucugcacauugguuaggcugggcuggguuagacccucgg

SEQ ID NO: 127 - RNA, 21 nt, Mus musculus
uuccuaugcauauacuucuuu

SEQ ID NO: 128 - RNA, 72 nt, Mus musculus
guuccuuuuuccuaugcauauacuucuuuguggaucuggucuaaagagguauagcgcaugggaagauggagc

SEQ ID NO: 129 - RNA, 22 nt, Mus musculus
acucaaacugggggcucuuuug

SEQ ID NO: 130 - RNA, 82 nt, Mus musculus
cagccugugauacucaaacugggggcucuuuuggauuuucaucggaagaaaagugccgccagguuuugagugucaccgguug

SEQ ID NO: 131 - RNA, 21 nt, Mus musculus
gaacggcgucaugcaggaguu

SEQ ID NO: 132 - RNA, 97 nt, Mus musculus
caguguagugagaaguuggggggugggaacggcgucaugcaggaguugauugcacagccauucagcuccuauaugaugccuuucuucacccccuuca

SEQ ID NO: 133 - RNA, 23 nt, Mus musculus
agguuacccgagcaacuuugcau

SEQ ID NO: 134 - RNA, 79 nt, Mus musculus
ugguacucggagagagguuacccgagcaacuuugcaucuggaggacgaauguugcucggugaaccccuuuucgguauca

SEQ ID NO: 135 - RNA, 22 nt, Homo sapiens
uacaguagucugcacauugguu

(19) United States
(12) Patent Application Publication
(10) Pub. No.: US 2009/0258928 A1
(43) Pub. Date: Oct. 15, 2009

(54) METHODS AND COMPOSITIONS FOR DIAGNOSING AND MODULATING HUMAN PAPILLOMAVIRUS (HPV)

(75) Inventors: Sylvie Beaudenon-Huibregtse, Austin, TX (US); Laimonis Laimins, Chicago, IL (US); Laura Langfield, Austin, TX (US); Gu Li, Evanston, IL (US); Emmanuel Labourier, Austin, TX (US)

Correspondence Address: Fullbright & Jaworski L.L.P., 600 Congress Avenue, Suite 2400, Austin, TX, 78701, US

(73) Assignees: Asuragen, Inc., Austin, TX (US); Northwestern University, Evanston, IL (US)

(21) Appl. No.: 12/420,634

(22) Filed: Apr. 8, 2009

Related U.S. Application Data
(60) Provisional application No. 61/043,339, filed on Apr. 8, 2008.

(51) Int. Cl. A61K 31/7088; C12N 5/06; C12Q 1/68; C12Q 1/70

(52) U.S. Cl. 514/44 A; 435/375; 435/6; 435/5

(57) ABSTRACT

The present invention concerns methods and compositions for treating a patient having, suspected of having, or at risk of obtaining a HPV infection.

This application claims priority to U.S. Provisional Application Ser. No. 61/043,339 filed Apr. 8, 2008, which is incorporated herein by reference in its entirety.

### BACKGROUND OF THE INVENTION

I. Field of the Invention

The present invention relates generally to the field of molecular biology. More particularly, it concerns methods and compositions involving microRNA (miRNAs) molecules. Certain aspects of the invention include applications for miRNAs in assessing, diagnosing, and/or treating human papillomavirus-associated diseases.

II. Background

The human papillomavirus (HPV) is now recognized as the major cause of cervical cancer and has been shown to be required for nearly all cases of the disease (Walboomers et al., 1999; Cogliano et al., 2005; Bosch and de Sanjose, 2007). In 2007, an estimated 11,000 women were diagnosed with cervical cancer and almost 4,000 died from the disease (Jemal et al., 2007). HPV has also been implicated in cancers of the oropharynx, including the soft palate, tongue, tonsils, esophagus, and larynx (D'Souza et al., 2007). Currently, no cure is available for HPV infections, but several treatment options are available.

miRNAs are short RNA molecules (16-29 nucleotides in length) that arise from longer precursors, which are transcribed from non-protein-encoding genes. See review of Carrington et al. (2003) and the miRBase database of the Welcome Trust Sanger Institute miRBase (which can be found on the internet at microrna.sanger.ac.uk/). miRNAs are misregulated in several diseases, and recent evidence indicates that some miRNAs have a role in the development of cancer, by acting as oncogenes or tumor suppressors (Calin and Croce, 2006; Esquela-Kerscher and Slack, 2006; He et al., 2007; Zhang et al., 2007). Expression levels of numerous miRNAs are associated specifically with various cancers (reviewed in Esquela-Kerscher and Slack, 2006; Calin and Croce, 2006). miRNAs have also been implicated in regulating cell growth, cell proliferation, and cell and tissue differentiation—cellular processes that are associated with the development of cancer (Harfe, 2005; Sevignani et al., 2006; Wiemer, 2007). Recent studies suggest that microRNAs (miRNAs) have a role in viral pathogenesis (Scaria et al., 2006; Scaria et al., 2007). Indeed, several lines of evidence suggest that host miRNAs exert substantial influence on viral tropism and viral life cycle, and therefore on viral evolution and pathogenesis. By using the cellular machinery for their survival and propagation, viruses are susceptible to host gene-regulatory mechanisms, including host miRNA-mediated post-transcriptional regulation. Lecellier et al. (2005) reported that hsa-miR-32 restricts the accumulation of the retrovirus primate foamy virus type 1 (PFV-1) in human cells, illustrating the role of miRNAs in antiviral defense. In contrast, Jopling et al. (2005) reported that a liver-specific miRNA, hsa-miR-122a, promotes accumulation of hepatitis C-encoded viral RNA in liver cells, suggesting a role in the tropism of hepatitis C virus for liver cells. Although no HPV-encoded miRNA has yet been reported (Cai et al., 2006), several RNA and DNA viruses have been shown to encode miRNAs.

There is a need for additional compositions and methods for the diagnosis and/or treatment of HPV infection.

### SUMMARY OF THE INVENTION

The present invention provides additional compositions and methods for the treatment of HPV infection by identifying miRNAs that are differentially expressed or mis-regulated in various states of diseased, normal, precancerous, and/or abnormal tissues, including but not limited to HPV infected tissue or cells. Further, the invention describes a method for treating HPV infection based on administering selected miRNAs or miRNA inhibitors to a patient, tissue, or cells at risk of infection, suspected of being infected, or infected with HPV.

The term “miRNA” or “miR” is used according to its ordinary and plain meaning and refers to a microRNA molecule found in eukaryotes that is involved in RNA-based gene regulation. See, e.g., Carrington et al., 2003, which is hereby incorporated by reference. The term will be used to refer to the single-stranded RNA molecule processed from a precursor. Names of miRNAs and their sequences related to the present invention are provided herein.

miR sequences can be used to evaluate cells and/or tissue for the possibility of a condition associated with HPV infection, particularly those conditions that will result in uncontrolled or hyperactive cell divisions and lead to the development of a disease or a pathological condition. Hyperproliferative conditions include benign lesions, precancerous lesions, and cancers.

In certain aspects, an miRNA that is differentially expressed between HPV infected cell and a normal cell is administered to a patient having or suspected of having HPV infection. In certain aspects, an inhibitor of a miRNA that is differentially expressed between HPV infected cell and a normal cell is administered to a patient having, suspected of having, or at risk of HPV infection.

Embodiments of the invention include methods of modulating cellular or viral nucleic acids and the processing of these nucleic acids comprising administering to the cell, tissue, or subject an amount of an isolated nucleic acid or mimetic thereof comprising all or part of an miRNA nucleic acid sequence, mimetic, or inhibitor sequence in an amount sufficient to modulate the processing of a cellular or viral nucleic acid by positive or negative modulation. Modulation includes one or more of modulating transcription, mRNA levels, mRNA translation, and/or protein levels in a cell, tissue, or organ. In certain aspects the expression of a gene or level of a gene product, such as mRNA or encoded protein, is down-regulated or up-regulated. A “therapeutic nucleic acid sequence” includes nucleic acids that positively or negatively modulate the processing of a cellular or viral nucleic acid. A therapeutic nucleic acid may include the full length precursor sequence of a miRNA identified herein, or a complement thereof. In certain aspects a therapeutic nucleic acid can comprise all or part of a processed (i.e., mature) miRNA sequence or complement thereof, as well as 5, 6, 7, 8, 9, 10, 11, 12, 13, 14, 15, 16, 17, 18, 19, 20, 21, 22, 23, 24, 25, 26, 27, 28, 29 or more nucleotides of a precursor miRNA or its processed sequence, or complement thereof, including all ranges and integers there between. In certain embodiments, the therapeutic nucleic acid sequence contains a full-length processed miRNA sequence described herein or a complement thereof. In still further aspects, the therapeutic nucleic acid comprises about, at least, or at most a 5, 6, 7, 8, 9, 10, 11, 12, 13, 14, 15, 16, 17, 18, 19, 20, 21, 22, 23, 24, 25, 50 nucleotide segment (including all ranges and integers there between) or complementary segment of a miRNA described herein that is at least 75, 80, 85, 90, 95, 98, 99 or 100% identical to SEQ ID NO:1 to SEQ ID NO:135. In certain aspects, a subset of these miRNAs will be used that include some but not all of the miRNA described herein. In certain aspects, 1, 2, 3, 4, 5, 6, 7, 8, 9, 10, 11, 12, 13, 14, 15, 16, 17, 18, 19, 20 or more miRNAs or segments thereof (including complementary sequences) will be included with all other miRNA sequences excluded. In certain aspects, subsets of these miRNAs will be used that include some but not all of the listed miRNA sequences, segments, or complements.

In certain aspects the invention includes methods of modulating Human Papillomavirus (HPV) infection of a cell comprising administering to the cell an amount of an isolated (a) nucleic acid inhibitor of hsa-miR-199a, hsa-miR-199a-AS, hsa-miR-214, hsa-miR-145, hsa-miR-143, hsa-miR-151, hsa-miR-379, hsa-miR-30a_{—}5p, hsa-miR-200a, hsa-miR-130a, hsa-miR-197, hsa-miR-181b, hsa-miR-503, and/or hsa-miR-130b; or (b) nucleic acid having a hsa-miR-503, hsa-miR-194, hsa-miR-491, hsa-miR-224, hsa-miR-96, hsa-miR-132, hsa-miR-30e-5p, and/or ambi-miR-7027 activity; in an amount sufficient to modulate HPV infection. In a further aspect, a hsa-miR-503 is administered to the cell or subject. In still a further aspect, an inhibitor of hsa-miR-379, hsa-miR-30a_{—}5p, and/or hsa-miR-200a is administered to the cell or subject, in certain instances the cell or subject is suspected of or at risk of an oncogenic HPV infection. Typically, a cell or subject can have, be suspected of having, or be at risk of developing a HPV infection. In certain aspects the HPV infection is associated with a precancerous or cancerous condition. A precancerous condition can include, but is not limited to, cervical intraepithelial neoplasia (CIN). The cell can be, but is not limited to, a cancer cell or a keratinocyte. In certain aspects an HPV infection is prevented, ameliorated, reduced, or eliminated.

In certain embodiments a therapeutic nucleic acid is a recombinant nucleic acid, such as RNA, DNA, or RNA/DNA hybrid. A recombinant nucleic acid of the invention can be comprised in a miRNA expression cassette, which can be further comprised in a viral vector, or plasmid DNA vector. Typically a viral vector is administered at a dose of 1×10⁵ to 1×10¹⁴ viral particles per dose or a plasmid DNA vector is administered at a dose of 100 mg per patient to 4000 mg per patient.

In certain aspects the therapeutic nucleic acid is a synthetic nucleic acid. Typically a nucleic acid is administered at a dose of 0.01 mg/kg of body weight to 10 mg/kg of body weight. The nucleic acid can be administered topically or by other administration methods well known to those skilled in the art. In certain aspects the nucleic acid is comprised in a pharmaceutical formulation, such as a lipid composition or a nanoparticle composition and/or biocompatible and/or biodegradable molecule composition.

In still a further aspect, 2, 3, 4, 5, 6, or more therapeutic nucleic acids may be administered together or sequentially. Typically if the nucleic acids are administered together, they will be in a single composition.

In yet still further aspects a therapy will reduce the viability of the cell, reduce proliferation of the cell, reduce metastasis of the cell, or increase the cell's sensitivity to therapy.

In yet another embodiment the invention includes methods of treating a patient diagnosed with or suspected of having or at risk of being infected with HPV comprising the steps of: (a) administering to the patient an amount of an isolated therapeutic nucleic acid comprising a miRNA sequence in an amount sufficient to modulate a cellular or viral pathway, wherein (i) the nucleic acid is an inhibitor of hsa-miR-199a, hsa-miR-199a-AS, hsa-miR-214, hsa-miR-145, hsa-miR-143, hsa-miR-151, hsa-miR-379, hsa-miR-30a_{—}5p, hsa-miR-200a, hsa-miR-130a, hsa-miR-197, hsa-miR-181b, hsa-miR-503, and/or hsa-miR-130b; or (ii) the nucleic acid has hsa-miR-503, hsa-miR-194, hsa-miR-491, hsa-miR-224, hsa-miR-96, hsa-miR-132, hsa-miR-30e-5p, and/or ambi-miR-7027 activity; and (b) administering a second therapy, wherein the modulation of the cellular or viral pathway sensitizes the patient to a second therapy.

In still another aspect the invention includes methods of selecting a miRNA to be administered to a subject suspected of having, or having a propensity for developing a HPV infection comprising: (a) determining an expression profile of one or more miRNA selected from hsa-miR-199a, hsa-miR-199a-AS, hsa-miR-214, hsa-miR-145, hsa-miR-143, hsa-miR-151, hsa-miR-379, hsa-miR-30a_{—}5p, hsa-miR-200a, hsa-miR-130a, hsa-miR-197, hsa-miR-181b, hsa-miR-503, and/or hsa-miR-130b, hsa-miR-503, hsa-miR-194, hsa-miR-491, hsa-miR-224, hsa-miR-96, hsa-miR-132, hsa-miR-30e-5p, and/or ambi-miR-7027; (b) assessing the sensitivity of the subject to a therapeutic nucleic acid based on the expression profile; and (c) selecting one or more therapeutic nucleic acid for administration to the patient based on the assessed sensitivity. The method can further comprise treating the subject with 1, 2, 3, 4, 5, 6, 7, 8, 9, 10, or more therapeutic nucleic acids administered individually, or in one or more combinations.

Corresponding miRNA sequences that can be used in the context of the invention include, but are not limited to, all or a portion of those sequences in the sequence listing provided herein, as well as the miRNA precursor sequence, or complement of one or more of these miRNAs.

In some embodiments, it may be useful to know whether a cell expresses a particular miRNA endogenously or whether such expression is affected under particular conditions or when it is in a particular disease state, e.g., HPV infection. Thus, in some embodiments of the invention, methods include assaying a cell or a sample containing a cell for the presence of one or more miRNA. In certain aspects, the miRNA evaluated include those differentially expressed when the cell is infected or has been infected with HPV. Consequently, in some embodiments, methods include a step of generating a miRNA profile for a sample. The term “miRNA profile” refers to data regarding the expression pattern of miRNAs in the sample (e.g., one or more miRNA from Table 1-5). It is contemplated that the miRNA profile can be obtained using a set of miRNAs, using for example nucleic acid amplification or hybridization techniques well know to one of ordinary skill in the art. In certain embodiments, expression of one or more miRNA from Table 1-5 is evaluated or reduced prior to or after administration of a therapeutic miRNA.

In some embodiments of the invention, an miRNA profile is generated by steps that include one or more of: (a) labeling miRNA in the sample; (b) hybridizing miRNA to a number of probes, or amplifying a number of miRNA, and/or (c) determining miRNA hybridization to the probes or detecting miRNA amplification products, wherein a miRNA expression is evaluated. See U.S. Provisional Patent Applications 60/575,743 and 60/649,584, and U.S. patent application Ser. Nos. 11/141,707 and 11/855,792, all of which are hereby incorporated by reference.

It is specifically contemplated that miRNA profiles for patients, particularly those suspected of having a particular disease or condition, such as HPV infection or precancerous lesions associated with HPV infection, can be generated by evaluating one or more miRNA or set of miRNAs discussed in this application. The miRNA profile that is generated from the patient will be one that provides information regarding the particular disease, condition, or therapeutic target. In certain aspects, a party evaluating miRNA expression may prepare a recommendation, report and/or summary conveying processed or raw data to a diagnosing physician. In certain aspects, a miRNA profile can be used in conjunction with other diagnostic tests or therapies.

Still a further embodiment includes methods of treating a patient with a pathological condition related to exposure to HPV comprising one or more of step of (a) administering to the patient an amount of a therapeutic nucleic acid comprising all or part of a miRNA nucleic acid sequence or complement thereof in an amount sufficient to modulate the expression of one or more genes, mRNA, and/or protein expression; and (b) administering a second therapy, wherein the modulation of one or more genes, mRNA, and/or protein sensitizes the patient to the second therapy. A second therapy can include administration of a second therapeutic nucleic acid, or may include various standard therapies, chemotherapy, radiation therapy, drug therapy, immunotherapy, and the like. Embodiments of the invention may also include the determination or assessment of a gene expression profile for the selection of an appropriate therapy. A physician may choose to treat a HPV infection or associated precancer or cancer using therapeutic nucleic acids of the invention in combination with standard treatment such as chemotherapy, radiation therapy, medications (e.g., Imiquimod (Aldara), Podofilox (Condylox), Trichloroacetic acid, or TCA) and/or surgery (e.g., freezing with liquid nitrogen (cryotherapy), electrocautery, surgical excision, laser treatments) and/or other methods.

Certain aspects of the invention include methods of treating a subject with a condition related to HPV exposure comprising one or more of the steps of (a) determining an expression profile of one or more miRNA selected from those miRNA described herein (b) assessing the sensitivity or amenability of the subject to therapy based on the expression profile; (c) selecting a therapy based on the assessment of the patient in light of the miRNA profile; and (d) treating the subject using one or more selected therapy.

Because “cancer” refers to a class of diseases, it is unlikely that there will be a single treatment and aspects of the invention can be used to determine which treatment will be most effective or most harmful and provide a guide for the physician in evaluating, assessing and formulating a treatment strategy for a patient.

Therapeutic nucleic acids may also include various heterologous nucleic acid sequences, i.e., those sequences not typically found operatively coupled with miRNA in nature, such as promoters, enhancers, and the like. The therapeutic nucleic acid can be a recombinant nucleic acid, and can be a ribonucleic acid and/or a deoxyribonucleic acid. The recombinant nucleic acid may comprise a therapeutic nucleic acid expression cassette, i.e., a nucleic acid segment that expresses a therapeutic nucleic acid when introduce into an environment containing components for nucleic acid synthesis. In a further aspect, the expression cassette is comprised in a viral vector, or plasmid DNA vector or other therapeutic nucleic acid vector or delivery vehicle, including liposomes and the like. In certain aspects, viral vectors can be administered at 1×10², 1×10³, 1×10⁴ 1×10⁵, 1×10⁶, 1×10⁷, 1×10⁸, 1×10⁹, 1×10¹⁰, 1×10¹¹, 1×10¹², 1×10¹³, 1×10¹⁴ pfu or viral particle (vp). In still further aspects, a therapeutic nucleic acid or a DNA comprising a therapeutic nucleic acid of the invention can be administered at 0.001, 0.01, 0.1, 1, 10, 20, 30, 40, 50, 100, 200, 400, 600, 800, 1000, 2000, to 4000 ng, μg, or mg, including all values and ranges there between.

In certain aspects, the therapeutic nucleic acid is a synthetic nucleic acid. Moreover, nucleic acids of the invention may be fully or partially synthetic. In yet a further aspect, nucleic acids of the invention, including synthetic nucleic acid, can be administered at 0.001, 0.01, 0.1, 1, 10, 20, 30, 40, 50, 100, to 200 μg or mg per kilogram (kg) of body weight. Each of the amounts described herein may be administered over a period of time, including 0.5, 1, 2, 3, 4, 5, 6, 7, 8, 9, 10, minutes, hours, days, weeks, months or years, including all values and ranges there between.

In certain embodiments, administration of the composition(s) can be enteral or parenteral. In certain aspects, enteral administration is oral. In further aspects, parenteral administration is intralesional, intravascular, intracranial, intrapleural, intratumoral, intraperitoneal, intramuscular, intralymphatic, intraglandular, subcutaneous, topical, intrabronchial, intratracheal, intranasal, inhaled, or instilled. Compositions of the invention may be administered regionally or locally and not necessarily directly into a lesion.

A further embodiment of the invention includes methods of detecting HPV infection in a biological sample comprising evaluating expression levels of hsa-mir-199a, hsa-miR-199a-AS, hsa-miR-214, hsa-miR-145, hsa-miR-143, hsa-miR-151, hsa-miR-503, hsa-miR-379, hsa-miR-30a_{—}5p, hsa-miR-200a, or combinations thereof. In certain aspects the biological sample is from a mucous membrane, skin, cervix, anus, rectum, penis, vulva, or vagina. In further aspects the biological sample is a pap smear. An increase in the level of hsa-mir-199a, hsa-miR-199a-AS, hsa-miR-214, hsa-miR-145, hsa-miR-143, hsa-miR-151, hsa-miR-379, hsa-miR-30a_{—}5p, hsa-miR-200a expression is indicative of an oncogenic HPV infection. A decrease in the expression level of hsa-miR-503 is indicative of an oncogenic HPV infection. And a decrease in the expression level of hsa-miR-200a is indicative of a non-oncogenic HPV infection.

The present invention also concerns kits containing compositions of the invention or compositions to implement methods of the invention. In some embodiments, kits can be used to administer one or more therapeutic nucleic acid molecules. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more therapeutic nucleic acids related to miRNAs described herein and include all or part of an miRNA and/or synthetic miRNA molecules and/or miRNA inhibitors, or any range and combination derivable therein. In some embodiments, there are kits for evaluating miRNA activity in a cell and assessing the effectiveness of nucleic acid therapy.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20×, including all values and ranges there between, or more.

Kits for using therapeutic nucleic acids and/or miRNA inhibitors of the invention for therapeutic applications are included as part of the invention. Specifically contemplated are any such molecules corresponding to any miRNA effective against HPV infection, such as those discussed herein.

It is contemplated that any method or composition described herein can be implemented with respect to any other method or composition described herein and that different embodiments may be combined. It is specifically contemplated that any methods and compositions discussed herein with respect to therapeutic nucleic acids, miRNA molecules, and/or miRNA inhibitors may be implemented with respect to synthetic miRNAs. Nucleic acids may be exposed to conditions that allow it to become processed under physiological circumstances. The claims originally filed are contemplated to cover claims that are multiply dependent on any filed claim or combination of filed claims.

Any embodiment of the invention involving specific miRNAs by name is contemplated also to cover embodiments involving miRNAs whose sequences are at least 80, 81, 82, 83, 84, 85, 86, 87, 88, 89, 90, 91, 92, 93, 94, 95, 96, 97, 98, 99% identical to the mature sequence of the specified miRNA. In other aspects miRNA of the invention may include additional nucleotides at the 5′, 3′, or both 5′ and 3′ ends of at least, at most or about 1, 2, 3, 4, 5, 6, 7, 8, 9, 10 or more nucleotides.

Embodiments of the invention include kits for analysis of a pathological sample by assessing miRNA profile for a sample comprising, in suitable container means, two or more miRNA probes and/or amplification primers, wherein the miRNA probes detect or primer amplify one or more miRNA described herein. The kit can further comprise reagents for labeling miRNA in the sample. The kit may also include the labeling reagents include at least one amine-modified nucleotide, poly(A) polymerase, and poly(A) polymerase buffer. Labeling reagents can include an amine-reactive dye.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” may be used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Comparison between array and qRT-PCR data. The graphs show the normalized expression levels of selected miRNAs as determined by Array or qRT-PCR at two time points, one corresponding to normal human keratinocyte (NHK) cells grown as monolayers (Mono) and the other corresponding to NHK cells after 48 hrs of methylcellulose-induced differentiation (48 hr). ANOVA p-values were obtained after comparison of miRNA expression levels between HPV-associated NHK and NHK at the two time points, mono (p) and 48 hr (*p). miR-199a, -145, -143, and -214 were below the background detection level in NHK samples at the two time points, as determined after array normalization and as indicated in the figures with a dashed line. The stippled lines shown in the qRT-PCR graphs for miR-199a, miR-143, and miR-503 indicate the limits of detection for that miRNA. Any point below the ACT value indicated by the stippled line corresponds to at least a 3.3 CT difference (10-fold) in raw CT values between a sample and the non template control.

### DETAILED DESCRIPTION OF THE INVENTION

The present invention is directed to compositions and methods relating to preparation and characterization of miRNAs, as well as use of miRNAs for therapeutic, prognostic, and diagnostic applications, particularly those methods and compositions related to assessing and/or identifying HPV infection and related conditions or diseases.

### I. Human Papillomavirus (HPV)

The human papillomavirus (HPV) is now recognized as the major cause of cervical cancer and has been shown to be required for nearly all cases of the disease (Walboomers et al., 1999; Cogliano et al., 2005; Bosch and de Sanjose, 2007). In 2007, an estimated 11,000 women were diagnosed with cervical cancer and almost 4,000 died from the disease (Jemal et al., 2007). HPV has also been implicated in cancers of the oropharynx, including the soft palate, tongue, tonsils, esophagus, and larynx (D'Souza et al., 2007).

HPVs are small, double stranded, circular DNA viruses of approximately 8,000 base pairs that specifically infect keratinocytes. Over one hundred different HPV types have been identified and characterized. About half of the types so far identified infect the anogenital tract (de Villiers et al., 2004). These sexually-transmitted viruses are further categorized into low-risk and high-risk types, according to their capacity for malignant conversion of the lesions that they induce (Clifford et al., 2003; Cox, 2006; Smith et al., 2007). Low-risk HPV types, (e.g., HPV6, HPV11) are associated with condyloma acuminata (genital warts), low grade squamous intraepithelial lesions (SILs) that do not generally progress toward cancer. In contrast, high-risk HPV types are associated with intraepithelial lesions that may progress to invasive carcinomas. HPV16 and HPV18 are among the high-risk HPV types most frequently observed in squamous cell cervical cancers and account for approximately 50% and 20%, respectively, of all squamous cell cervical cancers. Other high-risk HPV types (e.g., HPV31, HPV33, HPV35, HPV39, and others), account for an additional 25-30% of cervical cancers (Bosch et al., 1995; zur Hausen, 2002; Clifford et al., 2003). High-risk HPV types are also associated with 25% of head and neck tumors, in particular tumors of the mouth, tonsils, esophagus and larynx (Gillison et al., 2000; Rose et al., 2006).

HPV infection is believed to occur through micro-wounds in the epithelium that expose the only dividing cells of the epithelium, the basal cells (Longworth and Laimins, 2004). Establishment of infection and maintenance of HPV genomes are associated with expression of early HPV proteins. HPV genomes are maintained as episomes (20-100 copies per cell) in infected basal cells, and their replication is synchronized with host cellular DNA replication (Stubenrauch and Laimins, 1999). As infected cells divide, viral genomes are distributed into daughter cells. One daughter cell then leaves the basal cell layer and migrates to upper layers of the epithelium while undergoing differentiation (Doorbar et al., 1991). Normally, uninfected daughter cells exit the cell cycle at this point, but HPV-infected cells continue to divide, due to action of the viral oncoprotein E7. Cellular differentiation of HPV-infected cells is accompanied by replication of the viral genome to approximately 1,000 copies per cell, expression of late viral proteins, assembly of viral capsid proteins, and release of infectious virions (Lambert, 1991; Frattini et al., 1996). In low grade cervical lesions, HPV genomes are present as episomes. However, in high grade lesions and carcinomas, HPV genomes are often integrated into the cellular genome (Doorbar, 2006).

Currently, no cure is available for HPV infections, but several treatment options are available. Low grade SILs, or cervical intraepithelial neoplasias (CIN), that do not generally progress toward cancer will frequently regress within a few months. So, physicians often choose no treatment in this situation, and patients will have frequent follow-up visits to monitor lesions. If lesions do not spontaneously regress, physicians may recommend treatment for external genital warts or for CINs. Available treatments include cryotherapy (freezing the abnormal cells with liquid nitrogen), conization to excise the abnormal areas (also known as a cone biopsy), laser vaporization or excision, and loop electrosurgical excision procedure (LEEP) (abnormal cells are removed with electrical current). In addition, topical chemical treatment may include Imiquimod (Aldara), a prescription medication applied as a cream that may enhance the immune system's ability to fight HPV or Podofilox (Condylox), also applied as a cream, which may destroy the genital wart tissue. Physicians frequently recommend removal of high grade pre-malignant lesions, because these may develop into cancerous tumors.

Current treatments include cryotherapy, electrocautery, surgical removal, or laser surgery. All these procedures have failure rates in the range of 5 to 20%, and cellular changes may persist after treatment, leading to recurrences and repetitive interventions. Adjunctive agents, such as interferon, have not proved to be very effective, have potential adverse side-effects, and are costly. Therefore, additional therapies for treatment of HPV infection are needed.

Recent studies suggest that microRNAs (miRNAs) have a role in viral pathogenesis (Scaria et al., 2006; Scaria et al., 2007). miRNAs have been implicated in regulating cell growth, cell proliferation, and cell and tissue differentiation—cellular processes that are associated with the development of cancer (Harfe, 2005; Sevignani et al., 2006; Wiemer, 2007). Indeed, several lines of evidence suggest that host miRNAs exert substantial influence on viral tropism and viral life cycle, and therefore on viral evolution and pathogenesis. By using the cellular machinery for their survival and propagation, viruses are susceptible to host gene-regulatory mechanisms, including host miRNA-mediated post-transcriptional regulation. Lecellier et al. (2005) reported that hsa-miR-32 restricts the accumulation of the retrovirus primate foamy virus type 1 (PFV-1) in human cells, illustrating the role of miRNAs in antiviral defense. In contrast, Jopling et al. (2005) reported that a liver-specific miRNA, hsa-miR-122a, promotes accumulation of hepatitis C-encoded viral RNA in liver cells, suggesting a role in the tropism of hepatitis C virus for liver cells. Although no HPV-encoded miRNA has yet been reported (Cai et al., 2006), several RNA and DNA viruses have been shown to encode miRNAs.

The development of organotypic tissue culture systems for human keratinocytes has enabled in vitro replication of the differentiation program of keratinocytes (Hummel et al., 1992; Meyers et al., 1992; Meyers, 1996; Meyers et al., 1997), thus enabling the study of HPV infection in vitro. These systems now enable the study of mechanisms that control viral DNA replication and gene expression and constitute unique models for elucidating HPV pathogenicity. The current invention provides additional compositions and methods for treating and investigating HPV and its resulting pathology by describing miRNAs that are altered in human cells, e.g., keratinocytes, during HPV infection. These miRNAs, alone or in combination with other modalities, represent novel therapeutics for treatment of HPV infection.

### II. Therapeutic Methods

Certain embodiments of the invention concern nucleic acids that perform the activities of or inhibit endogenous miRNAs, mRNA or other cellular components when introduced into cells. In certain aspects, therapeutic nucleic acids (also referred to as nucleic acids) can be synthetic, non-synthetic, or a combination of synthetic and non-synthetic miRNA sequences. Sequence-specific miRNA inhibitors can be used to inhibit sequentially or in combination the activities of one or more miRNAs in cells, as well those genes and associated pathways (including those pathways modified or used by viral components) modulated by the miRNA.

The present invention concerns, in certain aspects, short nucleic acid molecules (therapeutic nucleic acids) that function as miRNAs or as inhibitors of miRNA in a cell. The term “short” refers to a length of a single polynucleotide that is 10, 15, 16, 17, 18, 19, 20, 21, 22, 23, 24, 25, 50, 100, or 150 nucleotides or fewer, including all integers or ranges derivable there between. The nucleic acid molecules are typically synthetic. The term “synthetic” refers to nucleic acid molecule that is isolated and not produced naturally in a cell. In certain aspects all or part of the sequence and/or chemical structure deviates from a naturally-occurring nucleic acid molecule (e.g., an endogenous precursor miRNA or miRNA molecule).

In certain aspects, nucleic acids of the invention do not have an entire sequence that is identical or complementary to a sequence of a naturally-occurring nucleic acid (e.g., miRNA), such molecules may encompass all or part of a naturally-occurring sequence or a complement thereof. It is contemplated, however, that a synthetic nucleic acid administered to a cell may subsequently be modified or altered in the cell such that its structure or sequence is the same or substantially the same as all or part of a non-synthetic or naturally occurring miRNA, such as a mature miRNA sequence. For example, a synthetic nucleic acid may have a sequence that differs from the sequence of a precursor miRNA, but that sequence may be altered once in a cell to be the same as an endogenous, processed miRNA or an inhibitor thereof.

The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many aspects of the invention, a nucleic acid is isolated by virtue of it having been synthesized in vitro separate from endogenous nucleic acids in a cell. It will be understood, however, that isolated nucleic acids may be subsequently mixed or pooled together.

In certain aspects, synthetic miRNA of the invention are RNA or RNA analogs. miRNA inhibitors may be DNA and/or RNA, or analogs thereof. miRNA and miRNA inhibitors of the invention are collectively referred to as “synthetic nucleic acids.”

In some embodiments, a therapeutic nucleic acid can have a miRNA or a synthetic miRNA sequence of between 10 and 200 to 17 and 130 residues, including all values and ranges there between. The present invention concerns miRNA or synthetic miRNA molecules that are, are at least, or are at most 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 145, 150, 160, 170, 180, 190, 200 or more residues in length, including any integer or any range there between.

In certain aspects, synthetic nucleic acids have (a) a “miRNA region” whose sequence or binding region from 5′ to 3′ is identical or complementary to all or a segment of a mature miRNA sequence, and (b) a “complementary region” whose sequence from 5′ to 3′ is between 60% and 100% complementary to the miRNA sequence in (a). In certain embodiments, these synthetic nucleic acids are also isolated, as defined above. The term “miRNA region” refers to a region on the synthetic nucleic acid that is at least 75, 80, 85, 90, 95, or 100% identical, including all integers there between, to the entire sequence of a mature, naturally occurring miRNA sequence or a complement thereof. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA or complement thereof.

The term “complementary region” or “complement” refers to a region of a nucleic acid or mimetic that is or is at least 60% complementary to the mature, naturally occurring miRNA sequence. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are synthetic nucleic acids that are miRNA inhibitors. A miRNA inhibitor is between about 10 to 30 or 17 to 25 nucleotides in length and comprises a 5′ to 3′ sequence that is at least 90% complementary to the 5′ to 3′ sequence of a mature miRNA. In certain embodiments, a miRNA inhibitor molecule is 10, 15, 16, 17, 18, 19, 20, 21, 22, 23, 24, or 25 nucleotides in length, or any range or value there between. Moreover, an miRNA inhibitor may have a sequence (from 5′ to 3′) that is or is at least 70, 75, 80, 85, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. A portion of a nucleic acid sequence can be altered so that it comprises an appropriate percentage of complementarity to the sequence of a mature miRNA.

In some embodiments of the invention a synthetic miRNA contains one or more design element(s). These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or, (iii) noncomplementarity between one or more nucleotides in the last 1 to 5 residues at the 3′ end of the complementary region and the corresponding nucleotides of the miRNA region. A variety of design modifications are known in the art, see below.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′oxygen-methyl), DMTO (4,4′-dimethoxytrityl with oxygen), fluoroscein, a thiol, or acridine, though other replacement groups are well known to those of skill in the art and can be used as well. This design element can also be used with a miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there are one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me modification, a 2′F modification, a 2′H modification, a 2′amino modification, a 4′thioribose modification or a phosphorothioate modification on the carboxy group linked to the carbon at position 6′ or combinations thereof. In further embodiments, there are one or more sugar modifications in the first or last 2 to 4 residues of the complementary region or the first or last 4 to 6 residues of the complementary region. This design element can also be used with a miRNA inhibitor. Thus, a miRNA inhibitor can have this design element and/or a replacement group on the nucleotide at the 5′ terminus, as discussed above.

In other embodiments of the invention, there is a synthetic miRNA in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that therapeutic nucleic acids of the invention have one or more of the replacement, sugar modification, or noncomplementarity designs. In certain cases, synthetic nucleic acid molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there can be a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA or inhibitor region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

Methods of the invention include reducing or eliminating activity of one or more miRNAs in a cell comprising introducing into a cell a therapeutic nucleic acid, such as a miRNA inhibitor, (the description of miRNA, where appropriate, also will refer to a miRNA inhibitor); or supplying or enhancing the activity of one or more miRNAs in a cell. The present invention also concerns inducing certain cellular characteristics by providing to a cell a particular nucleic acid, such as a specific therapeutic nucleic acid molecule or a miRNA inhibitor molecule. The therapeutic nucleic acid or miRNA inhibitor need not be synthetic. They may have a sequence that is identical to a naturally occurring miRNA or they may not have any design modifications. In certain embodiments, the therapeutic nucleic acid and/or the miRNA inhibitor are synthetic, as discussed above.

The particular nucleic acid molecule provided to the cell is understood to correspond to a particular miRNA in the cell, and thus, the miRNA in the cell is referred to as the “corresponding miRNA.” In situations in which a named miRNA molecule is introduced into a cell, the corresponding miRNA will be understood to be the induced or inhibited miRNA function. It is contemplated, however, that the therapeutic nucleic acid introduced into a cell is not a mature miRNA but is capable of becoming or functioning as a mature miRNA under the appropriate physiological conditions. In cases in which a particular corresponding miRNA is being inhibited by a miRNA inhibitor, the particular miRNA will be referred to as the “targeted miRNA.” It is contemplated that multiple corresponding or targeted or combinations of miRNAs may be involved. In particular embodiments, more than one therapeutic nucleic acid is introduced into a cell. Moreover, in other embodiments, more than one miRNA inhibitor is introduced into a cell. Furthermore, a combination of therapeutic nucleic acid(s) and miRNA inhibitor(s) may be introduced into a cell. The inventors contemplate that a combination of therapeutic nucleic acids may act at one or more points in cellular pathways of cells and that such combination may have increased efficacy on the target cell while not adversely effecting normal or non-targeted cells. Thus, a combination of therapeutic nucleic acids may have a minimal adverse effect on a subject or patient while supplying a sufficient therapeutic effect, such as amelioration of a condition, growth inhibition of a cell, death of a targeted cell, alteration of cell phenotype or physiology, slowing of cellular growth, sensitization to a second therapy, sensitization to a particular therapy, and the like.

Methods include identifying a cell or patient in need of inducing those cellular characteristics. Also, it will be understood that an amount of a therapeutic nucleic acid that is provided to a cell or organism is an “effective amount,” which refers to an amount needed (or a sufficient amount) to achieve a desired goal, such as inducing a particular cellular characteristic(s) or modulating HPV infection or aberrant phenotypes resulting from HPV infection.

In certain aspects methods can include providing or introducing to a cell a nucleic acid molecule corresponding to a mature miRNA in the cell in an amount effective to achieve a desired physiological result. Moreover, methods can involve providing synthetic or nonsynthetic therapeutic nucleic acids. It is contemplated that in these embodiments, that methods may or may not be limited to providing only one or more synthetic molecules or only one or more nonsynthetic molecules. Thus, in certain embodiments, methods may involve providing both synthetic and nonsynthetic therapeutic nucleic acids. In this situation, a cell or cells are most likely provided a synthetic molecule corresponding to a particular miRNA and a nonsynthetic molecule corresponding to a different miRNA or an inhibitor thereof. Furthermore, any method articulated using a list of miRNA targets using Markush group language may be articulated without the Markush group language and a disjunctive article (i.e., or) instead, and vice versa.

In some embodiments, there is a method for reducing or inhibiting cell proliferation, or viral replication or propagation in a cell comprising introducing into or providing to the cell an effective amount of (i) a therapeutic nucleic acid or (ii) a synthetic or nonsynthetic molecule that corresponds to a miRNA sequence. In certain embodiments the methods involve introducing into the cell an effective amount of (i) a miRNA inhibitor molecule having a 5′ to 3′ sequence that is at least 90% complementary to the 5′ to 3′ sequence of one or more miRNA.

Certain aspects of the invention include methods of treating a pathologic condition, such as a HPV related precancer or cancer. In one aspect, the method comprises contacting a target cell with one or more nucleic acid comprising at least one nucleic acid segment having all or a portion of a miRNA sequence or a complement thereof. The segment may be 5, 6, 7, 8, 9, 10, 11, 12, 13, 14, 15, 16, 17, 18, 19, 20, 21, 22, 23, 24, 25, 30 or more nucleotides or nucleotide analog, including all integers there between. An aspect of the invention includes the modulation of gene expression, miRNA expression or function or mRNA expression or function within a target cell, such as a HPV infected cell.

Typically, an endogenous gene, miRNA or mRNA is modulated in the cell. In certain aspects, a therapeutic nucleic acid sequence comprises at least one segment that is at least 70, 75, 80, 85, 90, 95, or 100% identical in nucleic acid sequence to one or more miRNA or gene sequence or complement thereof. Modulation of the expression or processing of a gene, miRNA, or mRNA of the cell or a virus can be through modulation of the processing of a nucleic acid, such processing including transcription, transportation and/or translation with in a cell. Modulation may also be effected by the inhibition or enhancement of miRNA activity with a cell, tissue, or organ. Such processing may affect the expression of an encoded product or the stability of the mRNA.

It will be understood in methods of the invention that a cell or other biological matter such as an organism (including patients) can be provided a therapeutic nucleic acid corresponding to or targeting a particular miRNA by administering to the cell or organism a nucleic acid molecule that functions as the corresponding miRNA once inside the cell. Thus, it is contemplated that a nucleic acid is provided such that it becomes processed into a mature and active miRNA once it has access to the cell's processing machinery. In certain aspects, it is specifically contemplated that the miRNA molecule provided is not a mature molecule but a nucleic acid molecule that can be processed into the mature miRNA or its functional equivalent once it is accessible to processing machinery.

The term “nonsynthetic” in the context of miRNA means that the miRNA is not “synthetic,” as defined herein. Furthermore, it is contemplated that in embodiments of the invention that concern the use of synthetic miRNAs, the use of corresponding nonsynthetic miRNAs is also considered an aspect of the invention, and vice versa. It will be understood that the term “providing” an agent is used to include “administering” the agent to a patient.

In certain embodiments, methods also include targeting a miRNA to modulate in a cell or organism. The term “targeting a miRNA to modulate” means a nucleic acid of the invention will be employed so as to modulate the selected miRNA. In some embodiments the modulation is achieved with a synthetic or non-synthetic nucleic acid that corresponds to the targeted miRNA, which effectively provides the function of the targeted miRNA to the cell or organism (positive modulation). In other embodiments, the modulation is achieved with a miRNA inhibitor, which effectively inhibits the targeted miRNA in the cell or organism (negative modulation).

In certain methods of the invention, there is a further step of administering a therapeutic nucleic acid to a cell, tissue, organ, or organism (collectively “biological matter”) in need of treatment related to modulation of the targeted miRNA or in need of the physiological or biological results discussed herein (such as with respect to a particular cellular pathway involved in HPV infection or HPV life cycle). Consequently, in some methods of the invention there is a step of identifying a patient in need of treatment that can be provided by the therapeutic nucleic acids of the invention. It is contemplated that an effective amount of a therapeutic nucleic acid can be administered in some embodiments. In certain aspects, there is a therapeutic benefit conferred on the biological matter, where a “therapeutic benefit” refers to an improvement in the one or more conditions or symptoms associated with a disease or condition or an improvement in the prognosis, duration, or status with respect to the disease. It is contemplated that a therapeutic benefit includes, but is not limited to, a decrease in pain, a decrease in morbidity, a decrease in a symptom. For example, with respect to cancer, it is contemplated that a therapeutic benefit can be inhibition of tumor growth, prevention of metastasis, reduction in number of metastases, inhibition of cancer cell proliferation, induction of cell death in cancer cells, inhibition of angiogenesis near cancer cells, induction of apoptosis of cancer cells, reduction in pain, reduction in risk of recurrence, induction of chemo- or radiosensitivity in cancer cells, prolongation of life, and/or delay of death directly or indirectly related to cancer.

Furthermore, it is contemplated that the nucleic acid compositions may be provided as part of a therapy to a patient, in conjunction with traditional therapies or preventative agents. Moreover, it is contemplated that any method discussed in the context of therapy may be applied as preventatively, particularly in a patient identified to be potentially in need of the therapy or at risk of the condition or disease for which a therapy is needed.

In addition, methods of the invention concern employing one or more nucleic acid corresponding to a miRNA and a therapeutic drug. The nucleic acid can enhance the effect or efficacy of the drug, reduce any side effects or toxicity, modify its bioavailability, and/or decrease the dosage or frequency needed. In certain embodiments, the therapeutic drug is a cancer therapeutic. Consequently, in some embodiments, there is a method of treating a HPV related precancer or cancer in a patient comprising administering to the patient a cancer therapeutic (i.e., a second therapeutic) and an effective amount of at least one nucleic acid molecule that improves the efficacy of the cancer therapeutic or protects non-cancer cells. Cancer therapies also include a variety of combination therapies with both chemical and radiation based treatments.

Generally, inhibitors of miRNAs can be given to decrease the activity of a miRNA and further modulate a nucleic acid targeted by the miRNA. Similarly, nucleic acid molecules corresponding to the mature miRNA can be given to achieve the opposite effect as compared to when inhibitors of the miRNA are given. Methods of the invention are generally contemplated to include providing or introducing one or more different nucleic acid molecules corresponding to one or more different miRNA molecules. It is contemplated that the following, at least the following, or at most the following number of different nucleic acid or miRNA molecules may be provided or introduced: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, including any value or range derivable there between.

### III. Pharmaceutical Formulations and Delivery

Methods of the present invention include the delivery of an effective amount of a therapeutic nucleic acid or an expression construct comprising or encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease.

A. Administration

In certain embodiments, it is desired to kill cells, inhibit cell growth, inhibit metastasis, decrease tumor or tissue size, and/or reverse or reduce the malignant or disease phenotype of cells. The routes of administration will vary, naturally, with the location and nature of the lesion or site to be targeted, and include, e.g., intradermal, subcutaneous, regional, parenteral, intravenous, intramuscular, intranasal, systemic, and oral administration and formulation. Direct topical or perfusion of a therapeutic nucleic acid is specifically contemplated for discrete, solid, accessible precancers or cancers, or other accessible target areas. Local, regional, or systemic administration also may be appropriate.

In the case of surgical intervention, the present invention may be used preoperatively, to render an inoperable lesion subject to resection. Alternatively, the present invention may be used at the time of surgery, and/or thereafter, to treat residual or metastatic disease. For example, a resected tumor bed may be injected or perfused with a formulation comprising a therapeutic nucleic acid or combinations thereof. Administration may be continued post-resection, for example, by leaving a catheter implanted at the site of the surgery. Periodic post-surgical treatment also is envisioned. Continuous perfusion of an expression construct or a viral construct also is contemplated.

Continuous administration also may be applied where appropriate, for example, where a tumor or other undesired affected area is excised and the tumor bed or targeted site is treated to eliminate residual, microscopic disease. Delivery via syringe or catherization is contemplated. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well and often depend on the type of lesion, location, immune condition, target site, disease progression, and health and age of the patient. Certain tumor types will require more aggressive treatment. The clinician will be best suited to make such decisions based on the known efficacy and toxicity (if any) of the therapeutic formulations.

In certain embodiments, the lesion or affected area being treated may not, at least initially, be resectable. Treatments with compositions of the invention may increase the resectability of the lesion due to shrinkage at the margins or by elimination of certain particularly invasive portions. Following treatments, resection may be possible. Additional treatments subsequent to resection may serve to eliminate microscopic residual disease at the tumor or targeted site.

Treatments may include various “unit doses.” A unit dose is defined as containing a predetermined quantity of a therapeutic composition(s). The quantity to be administered, and the particular route and formulation, are within the skill of those in the clinical arts. A unit dose need not be administered as a single injection but may comprise continuous infusion over a set period of time. With respect to a viral component of the present invention, a unit dose may conveniently be described in terms of μg or mg of miRNA or miRNA mimetic. Alternatively, the amount specified may be the amount administered as the average daily, average weekly, or average monthly dose.

A therapeutic nucleic acid can be administered to the patient in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 10000□g or mg, or more, or any range derivable therein. Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area).

B. Injectable Compositions and Formulations

In some embodiments, the method for the delivery of a therapeutic nucleic acid or an expression construct encoding such or combinations thereof is via topical or systemic administration. However, the pharmaceutical compositions disclosed herein may also be administered parenterally, subcutaneously, directly, intratracheally, intravenously, intradermally, intramuscularly, or even intraperitoneally as described in U.S. Pat. Nos. 5,543,158; 5,641,515 and 5,399,363 (each specifically incorporated herein by reference in its entirety).

Injection of nucleic acids may be delivered by syringe or any other method used for injection of a solution, as long as the nucleic acid and any associated components can pass through the particular gauge of needle required for injection. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, mixtures thereof, and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid-based. In particular embodiments of the invention, a composition comprising a miRNA or an miRNA inhibitor is in a water-based formulation. In other embodiments, the formulation is lipid based.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral, intralesional, and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

As used herein, a “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human.

The nucleic acid(s) are administered in a manner compatible with the dosage formulation, and in such amount as will be therapeutically effective. The quantity to be administered depends on the subject to be treated, including, e.g., the aggressiveness of the disease or cancer, the size of any tumor(s) or lesions, the previous or other courses of treatment. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

C. Combination Treatments

In certain embodiments, the compositions and methods of the present invention involve a therapeutic nucleic acid, or expression construct encoding such. These compositions can be used in combination with a second therapy to enhance the effect of the miRNA therapy, or increase the therapeutic effect of another therapy being employed. These compositions would be provided in a combined amount effective to achieve the desired effect, such as the killing of a cancer cell and/or the inhibition of cellular hyperproliferation. This process may involve contacting the cells with the therapeutic nucleic acid or second therapy at the same or different time. This may be achieved by contacting the cell with one or more compositions or pharmacological formulation that includes or more of the agents, or by contacting the cell with two or more distinct compositions or formulations, wherein one composition provides (1) therapeutic nucleic acid; and/or (2) a second therapy. A second composition or method may be administered that includes a chemotherapy, radiotherapy, surgical therapy, immunotherapy or gene therapy.

It is contemplated that one may provide a patient with the miRNA therapy and the second therapy within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed, for example miRNA therapy is “A” and a second therapy is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the vector or any protein or other agent. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a second therapy, such as chemotherapy, radiotherapy, immunotherapy, surgical therapy or other gene therapy, is employed in combination with the miRNA therapy, as described herein.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

a. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents.

b. Antimetabolites

Antimetabolites disrupt DNA and RNA synthesis. Unlike alkylating agents, they specifically influence the cell cycle during S phase. They have been used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

c. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms, these compounds are administered through bolus injections intravenously at doses ranging from 25-75 mg/m² at 21 day intervals for adriamycin, to 35-100 mg/m² for etoposide intravenously or orally.

d. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

e. Nitrosureas

Nitrosureas, like alkylating agents, inhibit DNA repair proteins. They are used to treat non-Hodgkin's lymphomas, multiple myeloma, and malignant melanoma, in addition to brain tumors. Examples include carmustine and lomustine.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. Radiotherapy may comprise the use of radiolabeled antibodies to deliver doses of radiation directly to the cancer site (radioimmunotherapy). Once injected into the body, the antibodies actively seek out the cancer cells, which are destroyed by the cell-killing (cytotoxic) action of the radiation. This approach can minimize the risk of radiation damage to healthy cells.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment a specially made metal frame is attached to the head. Then, several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor or disease cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. Nos. 5,830,880 and 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). A non-limiting list of several known anti-cancer immunotherapeutic agents and their targets includes (Generic Name/Target) Cetuximab/EGFR, Panitumuma/EGFR, Trastuzumab/erbB2 receptor, Bevacizumab/VEGF, Alemtuzumab/CD52, Gemtuzumab ozogamicin/CD33, Rituximab/CD20, Tositumomab/CD20, Matuzumab/EGFR, Ibritumomab tiuxetan/CD20, Tositumomab/CD20, HuPAM4/MUC1, MORAb-009/Mesothelin, G250/carbonic anhydrase IX, mAb 8H9/8H9 antigen, M195/CD33, Ipilimumab/CTLA4, HuLuc63/CSI, Alemtuzumab/CD53, Epratuzumab/CD22, BC8/CD45, HuJ591/Prostate specific membrane antigen, hA20/CD20, Lexatumumab/TRAIL receptor-2, Pertuzumab/HER-2 receptor, Mik-beta-1/IL-2R, RAV12/RAAG12, SGN-30/CD30, AME-133v/CD20, HeFi-1/CD30, BMS-663513/CD137, Volociximab/anti-α5β1 integrin, GC1008/TGFβ, HCD122/CD40, Siplizumab/CD2, MORAb-003/Folate receptor alpha, CNTO 328/IL-6, MDX-060/CD30, Ofatumumab/CD20, and SGN-33/CD33. It is contemplated that one or more of these therapies may be employed with the miRNA therapies described herein.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

4. Gene Therapy

In yet another embodiment, a combination treatment involves gene therapy in which a therapeutic polynucleotide is administered before, after, or at the same time as one or more therapeutic miRNA. Delivery of a therapeutic polypeptide or encoding nucleic acid in conjunction with a miRNA may have a combined therapeutic effect on target tissues. A variety of proteins are encompassed within the invention, some of which are described below. Various genes that may be targeted for gene therapy of some form in combination with the present invention include, but are not limited to inducers of cellular proliferation, inhibitors of cellular proliferation, regulators of programmed cell death, cytokines and other therapeutic nucleic acids or nucleic acid that encode therapeutic proteins.

The tumor suppressor oncogenes function to inhibit excessive cellular proliferation. The inactivation of these genes destroys their inhibitory activity, resulting in unregulated proliferation. The tumor suppressors (e.g., therapeutic polypeptides) p53, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus may regulate Rb phosphorylation (Serrano et al., 1993; Serrano et al., 1995). Since the p16INK4 protein is a CDK4 inhibitor (Serrano, 1993), deletion of this gene may increase the activity of CDK4, resulting in hyperphosphorylation of the Rb protein. p16 also is known to regulate the function of CDK6.

p16INK4 belongs to a newly described class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p16INK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increased intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al., 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

This application incorporates U.S. application Ser. No. 11/349,727 filed on Feb. 8, 2006 claiming priority to U.S. Provisional Application Ser. No. 60/650,807 filed Feb. 8, 2005 herein by references in its entirety.

### IV. Therapeutic Nucleic Acids

Therapeutic nucleic acids typically include segments of sequence or complementary sequences to microRNA (“miRNA” or “miR”) molecules, which are generally 21 to 22 nucleotides in length, though lengths of 16 and up to 35 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enable them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) becomes part of a large complex to down-regulate a particular target gene. Examples of animal miRNAs include those that imperfectly basepair with the target, which halts translation (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through a RNA-induced silencing complex (RISC) (Denli et al., 2003).

In certain aspects, therapeutic nucleic acids of the invention are RNA or RNA analogs. miRNA inhibitors may be DNA or RNA, or analogs thereof. In other aspects, an miRNA inhibitor can be a protein or a polypeptide that interacts with an endogenous miRNA or processing.

In some embodiments, there is a synthetic or isolated miRNA having a length of between 17 and 130 residues. The present invention concerns synthetic miRNA molecules that are, are at least, or are at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 145, 150, 160, 170, 180, 190, 200 or more residues in length, including any integer or any range derivable therein.

In certain embodiments, therapeutic nucleic acids have (a) a “miRNA region” whose sequence from 5′ to 3′ is identical to all or a segment of a mature miRNA sequence, and (b) a “complementary region” whose sequence from 5′ to 3′ is between 60% and 100% complementary to the miRNA sequence. In certain embodiments, these synthetic miRNA are also isolated, as defined above. The term “miRNA region” refers to a region on the synthetic miRNA that is at least 75, 80, 85, 90, 95, or 100% identical, including all integers there between, to the entire sequence of a mature, naturally occurring miRNA sequence. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA. Alternatively, the miRNA region can comprise 18, 19, 20, 21, 22, 23, 24, 25, 26, 27 or more nucleotide positions in common with a naturally-occurring miRNA as compared by sequence alignment algorithms and methods well known in the art.

The term “complementary region” refers to a region of a synthetic miRNA that is or is at least 60% complementary to the mature, naturally occurring miRNA sequence that the miRNA region is identical to. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are therapeutic nucleic acids that are miRNA inhibitors. A miRNA inhibitor is between about 17 to 27 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, 25, 26, or 27 nucleotides in length, or any range derivable therein. Moreover, a miRNA inhibitor has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use a portion of the probe sequence that is complementary to the sequence of a mature miRNA as the sequence for a miRNA inhibitor. Moreover, that portion of the probe sequence can be altered so that it is still 90% complementary to the sequence of a mature miRNA.

In some embodiments, of the invention, a therapeutic nucleic acid contains one or more design elements. These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or, (iii) noncomplementarity between one or more nucleotides in the last 1 to 5 residues at the 3′ end of the complementary region and the corresponding nucleotides of the miRNA region.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′oxygen-methyl), DMTO (4,4′-dimethoxytrityl with oxygen), fluorescein, a thiol, or acridine, though other replacement groups are well known to those of skill in the art and can be used as well. This design element can also be used with a miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there are one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me modification, a 2′F modification, a 2′H modification, a 2′amino modification, a 4′thioribose modification or a phosphorothioate modification on the carboxy group linked to the carbon at position 6′ or combinations thereof. In further embodiments, there are one or more sugar modifications in the first or last 2 to 4 residues of the complementary region or the first or last 4 to 6 residues of the complementary region. This design element can also be used with a miRNA inhibitor. Thus, a miRNA inhibitor can have this design element and/or a replacement group on the nucleotide at the 5′ terminus, as discussed above.

In other embodiments of the invention, there is a synthetic miRNA in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there is a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

In some embodiments of the invention, methods and compositions involving miRNA may concern miRNA and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA-containing vectors, control nucleic acids, and other probes and primers. In many embodiments, miRNA are 19-24 nucleotides in length, while miRNA probes are 5, 10, 15, 19, 20, 25, 30, to 35 nucleotides in length, including all values and ranges there between, depending on the length of the processed miRNA and any flanking regions added. miRNA precursors are generally between 62 and 110 nucleotides in humans.

Nucleic acids of the invention may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or 110 contiguous nucleotides. It is further understood that the length of complementarity within a precursor miRNA or between a miRNA probe and a miRNA or a miRNA gene are such lengths. Moreover, the complementarity may be expressed as a percentage, meaning that the complementarity between a probe and its target is 90% identical or greater over the length of the probe. In some embodiments, complementarity is or is at least 90%, 95% or 100% identical. In particular, such lengths may be applied to any nucleic acid comprising a nucleic acid sequence identified in any of SEQ ID NOs disclosed herein.

The term “recombinant” may be used and this generally refers to a molecule that has been manipulated in vitro or that is a replicated or expressed product of such a molecule.

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary. miRNA probes or nucleic acids of the invention can include, can be or can be at least 60, 65, 70, 75, 80, 85, 90, 95, 96, 97, 98, 99 or 100% complementary to their target.

Nucleic acids of the invention may be made by any technique known to one of ordinary skill in the art, such as for example, chemical synthesis, enzymatic production or biological production. It is specifically contemplated that miRNA probes of the invention are chemically synthesized.

In some embodiments of the invention, miRNAs are recovered or isolated from a biological sample. The miRNA may be recombinant or it may be natural or endogenous to the cell (produced from the cell's genome). It is contemplated that a biological sample may be treated in a way so as to enhance the recovery of small RNA molecules such as miRNA. U.S. patent application Ser. No. 10/667,126 describes such methods and it is specifically incorporated by reference herein. Generally, methods involve lysing cells with a solution having guanidinium and a detergent.

A. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased, such as from C.B.S. Scientific Co., Inc. or Scie-Plas.

Methods may involve the use of organic solvents and/or alcohol to isolate nucleic acids, particularly miRNA used in methods and compositions of the invention. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column has worked particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for form a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried down and resuspended in a liquid and volume appropriate for subsequent manipulation.

B. Preparation of Nucleic Acids

Alternatively, nucleic acid synthesis is performed according to standard methods. See, for example, Itakura and Riggs (1980). Additionally, U.S. Pat. Nos. 4,704,362, 5,221,619, and 5,583,013 each describe various methods of preparing synthetic nucleic acids. Non-limiting examples of a synthetic nucleic acid (e.g., a synthetic oligonucleotide), include a nucleic acid made by in vitro chemically synthesis using phosphotriester, phosphite, or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al., 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Recombinant methods for producing nucleic acids in a cell are well known to those of skill in the art. These include the use of vectors (viral and non-viral), plasmids, cosmids, and other vehicles for delivering a nucleic acid to a cell, which may be the target cell (e.g., a cancer cell) or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

In certain embodiments, the present invention concerns nucleic acid molecules that are not synthetic. In some embodiments, the nucleic acid molecule has a chemical structure of a naturally occurring nucleic acid and a sequence of a naturally occurring nucleic acid, such as the exact and entire sequence of a single stranded primary miRNA (see Lee 2002), a single-stranded precursor miRNA, or a single-stranded mature miRNA. In addition to the use of recombinant technology, such non-synthetic nucleic acids may be generated chemically, such as by employing technology used for creating oligonucleotides.

C. Labels and Labeling Techniques

In some embodiments, the present invention concerns miRNA that are directly or indirectly labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In many embodiments of the invention, the label is non-radioactive. Generally, nucleic acids may be labeled by adding labeled nucleotides (one-step process) or adding nucleotides and labeling the added nucleotides (two-step process).

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide or nucleotides is catalytically added to a miRNA, and the unlabeled nucleotide is modified with a chemical moiety that enables it to be subsequently labeled. In embodiments of the invention, the chemical moiety is a reactive amine such that the nucleotide is an amine-modified nucleotide. Examples of amine-modified nucleotides are well known to those of skill in the art, many being commercially available such as from Ambion, Sigma, Jena Bioscience, and TriLink.

In contrast to labeling of cDNA during its synthesis, the issue for labeling miRNA is how to label the already existing molecule. The present invention concerns the use of an enzyme capable of using a di- or tri-phosphate ribonucleotide or deoxyribonucleotide as a substrate for its addition to a miRNA. Moreover, in specific embodiments, it involves using a modified di- or tri-phosphate ribonucleotide, which is added to the 3′ end of a miRNA. The source of the enzyme is not limiting. Examples of sources for the enzymes include yeast, gram-negative bacteria such as E. coli, Lactococcus lactis, and sheep pox virus.

Enzymes capable of adding such nucleotides include, but are not limited to, poly(A) polymerase, terminal transferase, and polynucleotide phosphorylase. In specific embodiments of the invention, a ligase is contemplated as not being the enzyme used to add the label, and instead, a non-ligase enzyme is employed.

Terminal transferase catalyzes the addition of nucleotides to the 3′ terminus of a nucleic acid.

Polynucleotide phosphorylase can polymerize nucleotide diphosphates without the need for a primer.

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phycoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP.

Examples of fluorescently labeled deoxyribonucleotides include Dinitrophenyl (DNP)-11-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP.

It is contemplated that nucleic acids may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and the other ligands, include ligands for an antibody.

A number of techniques for visualizing or detecting labeled nucleic acids are readily available. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996), spectroscopy; mass spectroscopy; radiological techniques; and mass balance techniques.

When two or more differentially colored labels are employed, fluorescent resonance energy transfer (FRET) techniques may be employed to characterize association of one or more nucleic acid. Furthermore, a person of ordinary skill in the art is well aware of ways of visualizing, identifying, and characterizing labeled nucleic acids, and accordingly, such protocols may be used as part of the invention. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

V. Evaluation of miRNA Levels

It is contemplated that a number of assays could be employed to analyze miRNAs, their activities, and their effects. Such assays include, but are not limited to, array hybridization, solution hybridization, nucleic amplification, polymerase chain reaction, quantitative PCR, RT-PCR, in situ hybridization, Northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Oligo Ligation Assay (OLA), hybridization, and array analysis.

U.S. patent application Ser. Nos. 11/141,707, filed May 31, 2005; 11/857,948, filed Sep. 19, 2007; 11/273,640, filed Nov. 14, 2005 and provisional patent application 60/869,295, filed Dec. 8, 2006 are incorporated by reference in their entirety.

A. Sample Preparation

While endogenous miRNA is contemplated for use with compositions and methods of the invention, recombinant miRNA—including nucleic acids that are complementary or identical to endogenous miRNA or precursor miRNA—can also be handled and analyzed as described herein. Samples may be biological samples, in which case, they can be from lavage, biopsy, fine needle aspirates, exfoliates, blood, sputum, tissue, organs, semen, saliva, tears, urine, cerebrospinal fluid, body fluids, hair follicles, skin, or any sample containing or constituting biological cells. In certain embodiments, samples may be, but are not limited to, fresh, frozen, fixed, formalin fixed, preserved, RNAlater preserved, paraffin embedded, or formalin fixed and paraffin embedded. Alternatively, the sample may not be a biological sample, but be a chemical mixture, such as a cell-free reaction mixture (which may contain one or more biological enzymes).

B. Differential Expression Analyses

Methods of the invention can be used to detect differences in miRNA expression or levels between two samples, or a sample and a reference (e.g., a tissue reference or a digital reference representative of a non-cancerous state). Specifically contemplated applications include identifying and/or quantifying differences between miRNA from a sample that is normal and from a sample that is not normal, between a cancerous condition and a non-cancerous condition, or between two differently treated samples (e.g., a pretreatment versus a posttreatment sample). Also, miRNA may be compared between a sample believed to be susceptible to a particular therapy, disease, or condition and one believed to be not susceptible or resistant to that therapy, disease, or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of a disease or condition of which a component is or may or may not be genetic or caused by a hyperproliferative or neoplastic cell or cells.

It is specifically contemplated that the invention can be used to evaluate differences between stages of disease, such as between hyperplasia, neoplasia, pre-cancer and cancer, or between a primary tumor and a metastasized tumor.

Phenotypic traits also include characteristics such as longevity, morbidity, susceptibility or receptivity to particular drugs or therapeutic treatments (drug efficacy), and risk of drug toxicity.

In certain embodiments, miRNA profiles may be generated to evaluate and correlate those profiles with pharmacokinetics. For example, miRNA profiles may be created and evaluated for patient tumor and blood samples prior to the patient's being treated or during treatment to determine if there are miRNAs whose expression correlates with the outcome of treatment. Identification of differential miRNAs can lead to a diagnostic assay involving them that can be used to evaluate tumor and/or blood samples to determine what drug regimen the patient should be provided. In addition, it can be used to identify or select patients suitable for a particular clinical trial. If a miRNA profile is determined to be correlated with drug efficacy or drug toxicity that may be relevant to whether that patient is an appropriate patient for receiving the drug or for a particular dosage of the drug.

In addition to the above assay, blood samples from patients can be evaluated to identify a disease or a condition based on miRNA levels, such as metastatic disease. A diagnostic assay can be created based on the profiles that doctors can use to identify individuals with a disease or who are at risk to develop a disease. Alternatively, treatments can be designed based on miRNA profiling. Examples of such methods and compositions are described in the U.S. Provisional Patent Application entitled “Methods and Compositions Involving miRNA and miRNA Inhibitor Molecules” filed on May 23, 2005, which is hereby incorporated by reference in its entirety.

C. Amplification

Many methods exist for evaluating miRNA levels by amplifying all or part of miRNA nucleic acid sequences such as mature miRNAs, precursor miRNAs, and primary miRNAs. Suitable nucleic acid polymerization and amplification techniques include reverse transcription (RT), polymerase chain reaction (PCR), real-time PCR (quantitative PCR (q-PCR)), nucleic acid sequence-base amplification (NASBA), ligase chain reaction, multiplex ligatable probe amplification, invader technology (Third Wave), rolling circle amplification, in vitro transcription (IVT), strand displacement amplification, transcription-mediated amplification (TMA), RNA (Eberwine) amplification, and other methods that are known to persons skilled in the art. In certain embodiments, more than one amplification method may be used, such as reverse transcription followed by real time PCR (Chen et al., 2005 and/or U.S. patent application Ser. No. 11/567,082, filed Dec. 5, 2006, which are incorporated herein by reference in its entirety).

A typical PCR reaction includes multiple amplification steps, or cycles that selectively amplify target nucleic acid species. A typical PCR reaction includes three steps: a denaturing step in which a target nucleic acid is denatured; an annealing step in which a set of PCR primers (forward and reverse primers) anneal to complementary DNA strands; and an elongation step in which a thermostable DNA polymerase elongates the primers. By repeating these steps multiple times, a DNA fragment is amplified to produce an amplicon, corresponding to the target DNA sequence. Typical PCR reactions include 20 or more cycles of denaturation, annealing, and elongation. In many cases, the annealing and elongation steps can be performed concurrently, in which case the cycle contains only two steps. Since mature miRNAs are single stranded, a reverse transcription reaction (which produces a complementary cDNA sequence) is performed prior to PCR reactions. Reverse transcription reactions include the use of, e.g., a RNA-based DNA polymerase (reverse transcriptase) and a primer.

In PCR and q-PCR methods, for example, a set of primers is used for each target sequence. In certain embodiments, the lengths of the primers depends on many factors, including, but not limited to, the desired hybridization temperature between the primers, the target nucleic acid sequence, and the complexity of the different target nucleic acid sequences to be amplified. In certain embodiments, a primer is about 15 to about 35 nucleotides in length. In other embodiments, a primer is equal to or fewer than 15, 20, 25, 30, or 35 nucleotides in length. In additional embodiments, a primer is at least 35 nucleotides in length.

In a further aspect, a forward primer can comprise at least one sequence that anneals to a target miRNA and alternatively can comprise an additional 5′ noncomplementary region. In another aspect, a reverse primer can be designed to anneal to the complement of a reverse transcribed miRNA. The reverse primer may be independent of the miRNA sequence, and multiple miRNAs may be amplified using the same reverse primer. Alternatively, a reverse primer may be specific for a miRNA.

In some embodiments, two or more miRNAs or nucleic acids are amplified in a single reaction volume or multiple reaction volumes. In certain aspects, one or more miRNA or nucleic may be used as a normalization control or a reference nucleic acid for normalization. Normalization may be performed in separate or the same reaction volumes as other amplification reactions. One aspect includes multiplex q-PCR, such as qRT-PCR, which enables simultaneous amplification and quantification of at least one miRNA of interest and at least one reference nucleic acid in one reaction volume by using more than one pair of primers and/or more than one probe. The primer pairs comprise at least one amplification primer that uniquely binds each nucleic acid, and the probes are labeled such that they are distinguishable from one another, thus allowing simultaneous quantification of multiple miRNAs. Multiplex qRT-PCR has research and diagnostic uses, including but not limited to detection of miRNAs for diagnostic, prognostic, and therapeutic applications.

A single combined reaction for q-PCR, may be used to: (1) decreased risk of experimenter error, (2) reduction in assay-to-assay variability, (3) decreased risk of target or product contamination, and (4) increased assay speed. The qRT-PCR reaction may further be combined with the reverse transcription reaction by including both a reverse transcriptase and a DNA-based thermostable DNA polymerase. When two polymerases are used, a “hot start” approach may be used to maximize assay performance (U.S. Pat. Nos. 5,411,876 and 5,985,619). For example, the components for a reverse transcriptase reaction and a PCR reaction may be sequestered using one or more thermoactivation methods or chemical alteration to improve polymerization efficiency (U.S. Pat. Nos. 5,550,044, 5,413,924, and 6,403,341).

To assess the expression of microRNAs, real-time RT-PCR detection can be used to screen nucleic acids or RNA isolated from samples of interest and a related reference such as normal adjacent tissue (NAT) samples.

A panel of amplification targets is chosen for real-time RT-PCR quantification. The selection of the panel or targets can be based on the results of microarray expression analyses, such as mirVana™ miRNA Bioarray V1, Ambion. In one aspect, the panel of targets includes one or more miRNA described herein. One example of a normalization target is 5S rRNA and others can be included. Reverse transcription (RT) reaction components are typically assembled on ice prior to the addition of RNA template. Total RNA template is added and mixed. RT reactions are incubated in an appropriate PCR System at an appropriate temperature (15-30° C., including all values and ranges there between) for an appropriate time, 15 to 30 minutes or longer, then at a temperature of 35 to 42 to 50° C. for 10 to 30 to 60 minutes, and then at 80 to 85 to 95° C. for 5 minutes, then placed on wet ice. Reverse Transcription reaction components typically include nuclease-free water, reverse transcription buffer, dNTP mix, RT Primer, RNase Inhibitor, Reverse Transcriptase, and RNA.

PCR reaction components are typically assembled on ice prior to the addition of the cDNA from the RT reactions. Following assembly of the PCR reaction components a portion of the RT reaction is transferred to the PCR mix. PCR reaction are then typically incubated in an PCR system at an elevated temperature (e.g., 95° C.) for 1 minute or so, then for a number of cycles of denaturing, annealing, and extension (e.g., 40 cycles of 95° C. for 5 seconds and 60° C. for 30 seconds). Results can be analyzed, for example, with SDS V2.3 (Applied Biosystems). Real-time PCR components typically include Nuclease-free water, MgCl₂, PCR Buffer, dNTP mix, one or more primers, DNA Polymerase, cDNA from RT reaction and one or more detectable label.

Software tools such as NormFinder (Andersen et al., 2004) are used to determine targets for normalization with the targets of interest and tissue sample set. For normalization of the real-time RT-PCR results, the cycle threshold (Cₜ) value (a log value) for the microRNA of interest is subtracted from the geometric mean Cₜ value of normalization targets. Fold change can be determined by subtracting the dCₜ normal reference (N) from the corresponding dCₜ sample being evaluated (T), producing a ddCₜ(T-N) value for each sample. The average ddCₜ(T-N) value across all samples is converted to fold change by 2^{ddCt}. The representative p-values are determined by a two-tailed paired Student's t-test from the dCₜ values of sample and normal reference.

D. Nucleic Acid Arrays

Certain aspects of the present invention concerns the preparation and use of miRNA arrays or miRNA probe arrays, which are ordered macroarrays or microarrays of nucleic acid molecules (probes) that are fully or nearly complementary or identical to a plurality of miRNA molecules or precursor miRNA molecules and are positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO 0138580; WO 0168255; WO 03020898; WO 03040410; WO 03053586; WO 03087297; WO 03091426; WO 03100012; WO 04020085; WO 04027093; EP 373 203; EP 785 280; EP 799 897 and UK 8 803 000; the disclosures of which are all herein incorporated by reference. Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

E. Hybridization

After an array or a set of miRNA probes is prepared and the miRNA in the sample is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

### VI. Kits

Any of the compositions or components described herein may be comprised in a kit. In a non-limiting example, reagents for isolating miRNA, labeling miRNA, and/or evaluating a miRNA population using an array, nucleic acid amplification, and/or hybridization can be included in a kit, as well reagents for preparation of samples from colon samples. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. In certain aspects, the kit can include amplification reagents. In other aspects, the kit may include various supports, such as glass, nylon, polymeric beads, magnetic beads, and the like, and/or reagents for coupling any probes and/or target nucleic acids. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits for implementing methods of the invention described herein are specifically contemplated. In some embodiments, there are kits for preparing miRNA for multi-labeling and kits for preparing miRNA probes and/or miRNA arrays. In these embodiments, kit comprise, in suitable container means, 1, 2, 3, 4, 5, 6, 7, 8, 9, 10, 11, 12 or more of the following: (1) poly(A) polymerase; (2) unmodified nucleotides (G, A, T, C, and/or U); (3) a modified nucleotide (labeled or unlabeled); (4) poly(A) polymerase buffer; and, (5) at least one microfilter; (6) label that can be attached to a nucleotide; (7) at least one miRNA probe; (8) reaction buffer; (9) a miRNA array or components for making such an array; (10) acetic acid; (11) alcohol; (12) solutions for preparing, isolating, enriching, and purifying miRNAs or miRNA probes or arrays. Other reagents include those generally used for manipulating RNA, such as formamide, loading dye, ribonuclease inhibitors, and DNase.

In specific embodiments, kits of the invention include an array containing miRNA probes, as described in the application. An array may have probes corresponding to all known miRNAs of an organism or a particular tissue or organ in particular conditions, or to a subset of such probes. The subset of probes on arrays of the invention may be or include those identified as relevant to a particular diagnostic, therapeutic, or prognostic application. For example, the array may contain one or more probes that is indicative or suggestive of (1) a disease or condition (colon cancer), (2) susceptibility or resistance to a particular drug or treatment; (3) susceptibility to toxicity from a drug or substance; (4) the stage of development or severity of a disease or condition (prognosis); and (5) genetic predisposition to a disease or condition.

For any kit embodiment, including an array, there can be nucleic acid molecules that contain or can be used to amplify a sequence that is a variant of, identical to or complementary to all or part of any of SEQ ID NOs described herein. Any nucleic acid discussed above may be implemented as part of a kit.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Such kits may also include components that facilitate isolation of the labeled miRNA. It may also include components that preserve or maintain the miRNA or that protect against its degradation. Such components may be RNase-free or protect against RNases. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following: Control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits of the invention. Such kits, however, are not limited to the particular items identified above and may include any reagent used for the manipulation or characterization of miRNA.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Keratinocyte Culture and Differentiation, HPV Transfection, and RNA Isolation

Normal human keratinocytes (NHK) were isolated from pooled, neonatal foreskins obtained following the circumcision of infant male patients. Isolation of NHK from foreskins was performed according to published methods (McLaughlin-Drubin et al., 2003; Fehrmann and Laimins, 2005; Wilson and Laimins, 2005). NHK were grown in keratinocyte growth medium (Clonetics®KGM®, cat. no. CC-3001, Lonza Research Products, Allendale, N.J.; USA) and maintained as monolayer cell cultures with changes in culture medium every other day. After reaching approximately 80% confluence, cells were passaged at a ratio of 1:5, as previously described (Wilson and Laimins, 2005).

Recombinant plasmids for transfection, containing the entire genomes of HPV11, HPV18, and HPV31, were supplied by the German Cancer Research Center (DKFZ, Heidelberg, Germany). HPV genomes were excised from recombinant plasmids, purified, and re-ligated. Re-ligated, circular HPV genomes were transfected into NHK in the presence of a plasmid carrying a gene for resistance to neomycin, as previously described (Wilson and Laimins, 2005). Transfected cells were selected using G418 (an antibiotic for the selection and maintenance of eukaryotic cells expressing the neo gene) also as described previously (Wilson and Laimins, 2005). Keratinocyte cell lines containing HPV genomes were maintained as monolayer cell cultures in E medium in the presence of mitomycin C-treated J2 3T3 fibroblast feeder cells and were passaged when cells reached approximately 80-90% confluence (Wilson and Laimins, 2005).

Viral genomes are maintained as free episomes in HPV-positive NHK. Furthermore, as a result of the cooperative action of the viral oncoproteins E6 and E7, HPV18- and HPV31-positive NHK (NHK18, NHK31) are immortal cell lines. In contrast, due to the low oncogenicity of HPV11, HPV11-positive NHK (NHK11) are not immortal and cannot be maintained in culture indefinitely. Cultures of NHK11 must be regularly renewed from frozen, low-passage stocks.

For induction of NHK cell differentiation, monolayer cultures of NHK, NHK31, NHK18, and NHK11 were transferred in suspension in semisolid E medium containing 1.5% methylcellulose, which induces epithelial differentiation and stimulates viral late functions, including genomic amplification and late transcription (Wilson and Laimins, 2005). Cells were harvested at 24 hr and 48 hr after induction of differentiation.

The cervical intraepithelial neoplasia (CIN)-derived cell line, CIN-612 clone 9E was established from a biopsy specimen of a patient with a low-grade cervical intraepithelial lesion (CIN-1) (Rader et al., 1990). CIN-612 9E cells harbor episomal copies (approximately 50 per cell) of HPV31 (subtype HPV31b), a type associated with cervical cancer. It has been reported that CIN-612 9E cells, when allowed to stratify in raft cultures, differentiate in a manner that is histologically similar to that seen in CIN-1 biopsy lesions (Bedell et al., 1991; Hummel et al., 1992). Therefore, these cells represent a control of choice for this study.

All cells were grown in 100 mm Petri dishes and were collected when cultures reached approximately 80-90% confluence. Cells were trypsinized, harvested, and pelleted as described previously (Wilson and Laimins, 2005). Total RNA was extracted from cell pellets using the mirVana™ miRNA Isolation Kit (cat. no. AM1560; Ambion, Inc., Austin, Tex., USA), according to the manufacturer's protocol. Purified total RNA from all samples was quantified using a NanoDrop® ND-1000 spectrophotometer (NanoDrop Technologies; Wilmington, Del., USA).

### Example 2

### Microrna Expression Profile for Normal Human Keratinocytes (NHK), HPV-Associated NHK, and HPV31-Associated Cell Line (CIN-612 9E)

The effect of HPV infection on the miRNA expression profile of its host cells, human keratinocytes, is currently unknown. Therefore, the inventors first established the miRNA expression profiles of NHK, HPV-associated NHK (NHK18, NHK31, and NHK11), and the HPV31-associated cell line, CIN-612 9E. Two independent cell cultures were profiled for each cell type, except for HPV11-associated NHK and CIN-612 9E, for which only one sample of each was profiled.

Cells were cultured and harvested as described above in Example 1. miRNA expression profiling was performed as previously described (Shingara et al., 2005) with the following modifications. To isolate miRNA fractions, total RNA samples (10 μg for each sample) were fractionated and purified using the flashPAGE™ fractionator and reagents (cat. no. AM13100; Ambion) according to the manufacturer's recommendations. Purified small RNAs were labeled with Cy5 (GE Healthcare Life Sciences; Piscataway, N.J., USA) and hybridized to mirVana miRNA Bioarrays V1 (Ambion) according to the manufacturer's instructions. The arrays contained 377 individual miRNA probes, including 281 human miRNAs from the miRBase Sequence Database (microrna.sanger.ac.uk) (Griffiths-Jones et al., 2006), 33 new human miRNAs (ambi-miRs) and 63 mouse or rat miRNAs from the miRBase Sequence Database. Following hybridization, the arrays were scanned using the Axon® GenePix 400B scanner and associated GenePix software (Molecular Devices Corporation; Sunnyvale, Calif., USA).

Array Data Processing. The background corrected array signals were normalized with the VSN method (Huber et al., 2002). VSN is a global normalization process that stabilizes the variance evenly across the entire range of expression. It involves calibration of signal followed by a transformation to a generalized natural logarithmic space in lieu of the traditional logarithm base 2 transformation. Absolute and differences in VSN transformed expression are denoted by H and ΔH, respectively, and were used for all subsequent data analyses. Differences in normalized expression values between samples (ΔH) can be transformed to a generalized fold change via exponentiation base e. These values will exhibit a compression for small differences in expression. For an overview of miRNA processing and analysis (Davison et al., 2006). For each array, the minimum observable threshold was determined by examining the foreground minus background median intensities for ‘EMPTY’ spots. The minimum threshold was defined as 5% symmetric mean plus the MAX standard deviations across all ‘EMPTY’ spots on individual array.

Statistical Analysis. As the experimental design contained two independent variables, the analysis was performed using a two-way ANOVA method analysis. Seventeen samples, corresponding to five distinct groups, were analyzed on the arrays. Each group contained two time points corresponding to monolayer (M) and 48 hr post-differentiation (48). Two replicates of each group of samples were analyzed for NHK cells (2×NHKM and 2×NHK48), NHK31 cells (2×NHK31M and 2×NHK3148), and NHK18 cells (2×NHK18M and 2×NHK1848) and NHK 11 at the 48 hr time point (2×NHK 1148) but only one sample of each group was available for the CIN-612-9E cell line (1×CIN-612-9EM and 1×CIN-612-9E48) and NHK11 cells (1×NHK11M). Statistical analysis of the data was performed using a fixed-effects model (Model I) two-way ANOVA using Partek Genomic Solutions 6.2 (Partek Inc., St. Louis, Mo., USA). In this model, cell type (NHK, NHK11, NHK18, NHK31, and CIN-612-9E) and treatment (mono and 48 hr) were treated as main effect factors, with interaction effects comprising all biologically relevant pair-wise interaction terms of cell type * treatment. Differentially expressed genes (DEG) were identified as follows (Draghici, 2003): (1) identify significant differences in means of the main factors and primary interaction term followed by (2) subsequent pair-wise t-tests with correction for multiple testing for all significant differences identified in (1) in order to identify the significant pair-wise interaction terms under the control of a “protected Least Significant Difference test” (LSD) to control the false discovery rate (FDR) at 0.05 (Benjamini, 1995). The FDR report lists the adjusted p-values for all main factors and interaction terms along with the respective number of features passing the threshold, based on the FDR method for multiple test correction (Benjamini, 1995).

Unsupervised clustering of samples and miRNA expression levels at the two time points (monolayer and 48 hr) showed a clear segregation between NHK and HPV-associated cell lines. The samples clustered first according to the absence or presence of an HPV type and then separated according to the oncogenic potential of the HPV type and the response to the treatment (induction of differentiation) of the different cell types. Importantly, the low grade HPV31-associated CIN1-derived cell line, CIN-612-9E, clustered together with the high-risk HPV type-associated cell lines (NHK31 and NHK18), clearly indicating that the CIN1-derived cell line and the HPV-immortalized NHKs share common characteristics. This observation underscores the validity of using HPV-stably transfected NHK as models to study miRNA expression changes occurring in early HPV-infected cells.

Results of the miRNA expression analyses are shown in Table 1. On average, 108 miRNAs were detected above background threshold signal (>3.40) in NHKM (Table 1) corresponding to 29% of the miRNA probes present on the array. These included 102 well characterized human miRNAs, three miRNAs identified in mouse or rat, and five new human miRNAs. In the HPV31-associated NHK (NHK31M), approximately 152 miRNAs were detected above background signal (>3.07) (Table 1), corresponding to 40% of the miRNA probes present on the array. These included 130 characterized miRNAs, fifteen mouse or rat miRNAs, and eight new human miRNAs. In the HPV18-associated NHK (NHK18M), approximately 153 miRNAs were detected above background signal (>3.00) (Table 1), corresponding to 41% of the probes present on the array. These included 137 characterized miRNAs, fifteen mouse or rat miRNAs, and seven new human miRNAs. In addition, approximately 130 miRNAs were detected above background signal in the HPV11-associated NHK sample (NHK11M), corresponding to 35% of the miRNA probes on the array. These included 119 characterized miRNAs, eleven mouse or rat miRNAs, and six new human miRNAs (Table 1). Finally, over 180 miRNAs were detected above background signal in the HPV31-associated CIN-612 9E cell line (CIN-612-9EM), corresponding to 48% of the miRNA probes present on the array. These included 150 characterized human miRNAs, fifteen mouse or rat miRNAs, and fourteen new human miRNAs.

TABLE 1
MicroRNA expression profiles for monolayer cell cultures of normal human keratinocytes (NHK), HPV-associated NHK
(NHK31, NHK18, NHK11), and HPV31-associated cell line (CIN-612-9E). M, monolayer cells; 48, cells at 48 hours post-
differentiation.

| | | | | | | | | | | CIN- | CIN- |
|---|---|---|---|---|---|---|---|---|---|---|---|
| | | | NHK | NHK | NHK | NHK | NHK | NHK | NHK | 612- | 612- |
| | | NHK M | 48 | 31M | 3148 | 18M | 1848 | 11M | 1148 | 9EM | 9E48 |
| | | Threshold Value | | | | | | | | | |
| | | 3.40 | 3.07 | 3.07 | 2.91 | 3.00 | 2.62 | 3.15 | 3.32 | 2.57 | 2.60 |
| | | miRNAs Above Threshold Value | | | | | | | | | |
| | | 108.20 | 140.03 | 151.78 | 157.16 | 153.25 | 173.81 | 130.2 | 143.46 | 181.2 | 172.3 |
| | | % of miRNAs Detected | | | | | | | | | |
| | | | 37.14 | 40.26 | 41.69 | 40.65 | 46.10 | | 38.05 | 48 | 46 |
| | | 28.70 | Mean | Mean | Mean | Mean | Mean | 35 | Mean | CIN- | CIN- |
| | miR | Mean | NHK | NHK | NHK | NHK | NHK | NHK | NHK | 612- | 612- |
| BA ID | Name | NHK M | 48 | 31M | 3148 | 18M | 1848 | 11M | 1148 | 9EM | 9E48 |
| BA10196 | Hsa_miR_205 | 10.65 | 10.18 | 10.22 | 10.19 | 10.38 | 9.94 | 9.92 | 10.03 | 10.40 | 10.40 |
| BA10199 | Hsa_miR_21 | 10.05 | 10.44 | 9.39 | 10.08 | 9.95 | 9.92 | 8.79 | 9.70 | 9.85 | 10.17 |
| BA10216 | Hsa_miR_23a | 9.68 | 9.38 | 8.96 | 9.07 | 9.60 | 9.21 | 9.01 | 8.72 | 9.17 | 9.05 |
| BA10102 | Hsa_let_7b | 9.49 | 8.62 | 8.47 | 8.34 | 8.84 | 8.41 | 8.67 | 8.56 | 8.68 | 8.50 |
| BA10101 | Hsa_let_7a | 9.39 | 9.40 | 9.00 | 9.32 | 9.21 | 9.08 | 7.69 | 8.95 | 9.32 | 9.17 |
| BA10193 | Hsa_miR_200c | 9.14 | 9.27 | 8.52 | 8.84 | 9.01 | 9.14 | 8.48 | 8.82 | 8.81 | 9.10 |
| BA10244 | Hsa_miR_31 | 9.14 | 9.24 | 9.01 | 9.10 | 8.89 | 8.99 | 8.81 | 9.27 | 9.12 | 9.33 |
| BA10217 | Hsa_miR_23b | 8.94 | 9.03 | 8.41 | 8.68 | 8.88 | 8.68 | 8.34 | 8.36 | 8.73 | 8.62 |
| BA10159 | Hsa_miR_16 | 8.92 | 8.70 | 8.57 | 8.88 | 8.86 | 9.08 | 7.90 | 8.07 | 8.86 | 9.21 |
| BA10103 | Hsa_let_7c | 8.88 | 8.81 | 8.25 | 8.47 | 8.47 | 8.27 | 7.12 | 8.23 | 8.74 | 8.30 |
| BA10227 | Hsa_miR_29a | 8.78 | 9.18 | 8.49 | 8.87 | 8.71 | 8.91 | 7.98 | 8.49 | 8.69 | 8.58 |
| BA10213 | Hsa_miR_222 | 8.66 | 8.17 | 8.24 | 8.29 | 8.44 | 8.12 | 8.14 | 7.95 | 8.70 | 8.38 |
| BA10218 | Hsa_miR_24 | 8.60 | 8.84 | 8.42 | 8.63 | 9.07 | 9.09 | 8.78 | 9.18 | 8.61 | 8.85 |
| BA10212 | Hsa_miR_221 | 8.45 | 8.69 | 8.17 | 8.86 | 8.52 | 8.74 | 7.67 | 8.53 | 8.72 | 8.80 |
| BA10222 | Hsa_miR_27a | 8.29 | 8.95 | 8.47 | 8.46 | 8.76 | 8.79 | 8.93 | 8.77 | 8.39 | 8.82 |
| BA10114 | Hsa_miR_106a | 8.18 | 7.82 | 8.42 | 7.94 | 8.02 | 7.79 | 7.50 | 7.46 | 8.32 | 8.03 |
| BA10104 | Hsa_let_7d | 8.14 | 8.50 | 7.96 | 8.31 | 8.04 | 8.00 | 6.79 | 7.68 | 8.31 | 8.01 |
| BA10246 | Hsa_miR_320 | 8.11 | 7.37 | 7.16 | 7.04 | 7.49 | 7.24 | 7.11 | 6.97 | 7.22 | 6.97 |
| BA10161 | Hsa_miR_17_5p | 7.96 | 7.56 | 8.23 | 7.70 | 8.04 | 7.69 | 7.39 | 7.12 | 8.34 | 7.87 |
| BA10301 | Hsa_miR_92 | 7.81 | 7.41 | 7.34 | 7.69 | 7.18 | 6.99 | 6.94 | 6.89 | 7.23 | 7.11 |
| BA10158 | Hsa_miR_15b | 7.80 | 6.93 | 7.34 | 7.23 | 7.60 | 7.08 | 6.15 | 5.97 | 7.92 | 7.77 |
| BA10220 | Hsa_miR_26a | 7.75 | 8.24 | 7.63 | 7.96 | 7.95 | 8.24 | 7.41 | 7.62 | 7.42 | 7.69 |
| BA10192 | Hsa_miR_200b | 7.74 | 8.22 | 6.80 | 7.80 | 7.43 | 7.63 | 6.96 | 7.22 | 7.20 | 7.53 |
| BA10122 | Hsa_miR_125b | 7.71 | 7.39 | 7.50 | 7.65 | 7.64 | 7.37 | 7.94 | 7.70 | 7.32 | 6.74 |
| BA10164 | Hsa_miR_181b | 7.53 | 6.21 | 6.40 | 6.12 | 6.73 | 6.09 | 6.17 | 5.82 | 6.41 | 6.17 |
| BA10140 | Hsa_miR_141 | 7.48 | 7.41 | 7.21 | 7.01 | 7.28 | 7.27 | 8.34 | 6.90 | 7.19 | 7.48 |
| BA10310 | mmu_miR_106a | 7.41 | 7.19 | 7.68 | 7.30 | 7.52 | 7.14 | 6.35 | 6.65 | 7.81 | 7.37 |
| BA10163 | Hsa_miR_181a | 7.39 | 6.41 | 6.46 | 6.20 | 6.70 | 6.22 | 7.15 | 6.30 | 6.27 | 6.29 |
| BA10464 | Hsa_miR_503 | 7.37 | 5.29 | 5.00 | 3.86 | 5.53 | 4.23 | 6.72 | 5.33 | 5.01 | 4.39 |
| BA10112 | Hsa_miR_103 | 7.36 | 7.09 | 7.07 | 7.23 | 7.50 | 7.64 | 7.19 | 7.06 | 7.52 | 7.63 |
| BA10128 | Hsa_miR_130a | 7.35 | 6.51 | 6.94 | 6.34 | 7.28 | 6.98 | 6.94 | 6.45 | 6.45 | 6.02 |
| BA10110 | Hsa_miR_100 | 7.32 | 6.42 | 6.90 | 6.67 | 7.12 | 6.69 | 6.97 | 6.56 | 6.75 | 6.32 |
| BA10194 | Hsa_miR_203 | 7.30 | 10.62 | 7.85 | 10.16 | 8.02 | 9.95 | 8.60 | 10.08 | 7.02 | 9.86 |
| BA10116 | Hsa_miR_107 | 7.24 | 7.09 | 6.95 | 7.20 | 7.48 | 7.57 | 7.22 | 7.08 | 7.43 | 7.44 |
| BA10210 | Hsa_miR_22 | 7.13 | 7.73 | 7.11 | 7.81 | 7.61 | 8.06 | 8.21 | 8.24 | 7.34 | 7.78 |
| BA10302 | Hsa_miR_93 | 7.12 | 6.98 | 7.14 | 7.58 | 7.35 | 7.63 | 6.60 | 6.72 | 7.51 | 7.80 |
| BA10189 | Hsa_miR_19b | 7.10 | 7.49 | 7.83 | 7.62 | 7.83 | 7.56 | 7.04 | 6.95 | 7.65 | 7.50 |
| BA10409 | Hsa_miR_193b | 7.08 | 7.66 | 5.84 | 7.46 | 6.61 | 7.55 | 7.28 | 7.46 | 5.79 | 7.36 |
| BA10121 | Hsa_miR_125a | 7.07 | 6.69 | 6.52 | 6.52 | 6.95 | 6.80 | 6.83 | 6.68 | 6.90 | 6.93 |
| BA10105 | Hsa_let_7e | 7.02 | 7.74 | 6.70 | 7.53 | 7.03 | 7.39 | 5.86 | 6.89 | 7.13 | 7.22 |
| BA10223 | Hsa_miR_27b | 6.96 | 7.56 | 7.61 | 7.23 | 7.66 | 7.51 | 7.77 | 7.43 | 7.67 | 7.71 |
| BA10238 | Hsa_miR_30a_5p | 6.90 | 7.39 | 7.19 | 7.75 | 7.59 | 7.89 | 7.02 | 7.24 | 7.28 | 7.64 |
| BA10107 | Hsa_let_7g | 6.87 | 7.32 | 6.44 | 7.18 | 6.38 | 6.61 | 3.86 | 6.40 | 6.81 | 6.55 |
| BA10190 | Hsa_miR_20a | 6.87 | 6.86 | 6.90 | 6.88 | 7.04 | 6.79 | 4.83 | 6.00 | 7.14 | 6.78 |
| BA10240 | Hsa_miR_30c | 6.85 | 7.16 | 6.86 | 7.49 | 7.09 | 7.51 | 6.55 | 6.74 | 6.91 | 7.27 |
| BA10176 | Hsa_miR_191 | 6.77 | 6.35 | 6.70 | 6.60 | 6.99 | 6.74 | 7.07 | 6.57 | 6.71 | 6.42 |
| BA10106 | Hsa_let_7f | 6.72 | 7.95 | 6.61 | 7.56 | 6.38 | 6.78 | 4.29 | 6.78 | 6.86 | 6.82 |
| BA10241 | Hsa_miR_30d | 6.69 | 7.02 | 6.56 | 7.27 | 6.77 | 7.24 | 6.42 | 6.78 | 6.87 | 7.39 |
| BA10108 | Hsa_let_7i | 6.68 | 6.66 | 6.85 | 7.12 | 7.13 | 7.12 | 5.40 | 6.48 | 7.05 | 6.65 |
| BA10191 | Hsa_miR_200a | 6.42 | 6.87 | 5.65 | 6.59 | 6.27 | 6.55 | 6.34 | 6.10 | 5.89 | 6.41 |
| BA10115 | Hsa_miR_106b | 6.37 | 6.27 | 6.56 | 6.63 | 6.44 | 6.55 | 6.22 | 5.73 | 6.56 | 6.57 |
| BA10228 | Hsa_miR_29b | 6.34 | 7.21 | 6.33 | 6.49 | 6.19 | 6.33 | 5.93 | 6.21 | 6.22 | 6.02 |
| BA10215 | Hsa_miR_224 | 6.34 | 7.63 | 6.21 | 7.16 | 5.92 | 7.33 | 5.91 | 6.60 | 6.18 | 6.87 |
| BA10290 | Hsa_miR_422b | 6.28 | 5.71 | 6.46 | 6.47 | 5.98 | 6.36 | 5.96 | 5.67 | 6.43 | 6.88 |
| BA10166 | Hsa_miR_182 | 6.26 | 6.62 | 5.49 | 6.06 | 6.29 | 6.39 | 5.99 | 6.16 | 5.88 | 6.22 |
| BA10264 | Hsa_miR_34a | 6.22 | 6.83 | 6.27 | 6.89 | 6.27 | 6.81 | 7.27 | 7.37 | 6.78 | 6.93 |
| BA10219 | Hsa_miR_25 | 6.16 | 6.45 | 5.83 | 6.76 | 6.16 | 6.62 | 4.27 | 5.36 | 6.44 | 6.52 |
| BA10129 | Hsa_miR_130b | 6.06 | 4.70 | 5.23 | 4.77 | 5.61 | 5.42 | 5.09 | 4.32 | 5.01 | 5.05 |
| BA10307 | Hsa_miR_99b | 6.02 | 6.03 | 5.90 | 6.27 | 6.31 | 6.51 | 5.94 | 5.97 | 6.53 | 6.42 |
| BA10455 | ambi_miR_7083 | 6.00 | 5.30 | 5.28 | 4.98 | 5.28 | 4.86 | 5.42 | 4.74 | 5.18 | 5.02 |
| BA10239 | Hsa_miR_30b | 5.90 | 7.25 | 5.42 | 7.38 | 5.42 | 6.33 | 4.72 | 6.00 | 5.89 | 6.54 |
| BA10181 | Hsa_miR_196a | 5.86 | 5.91 | 5.33 | 5.40 | 5.57 | 5.60 | 4.52 | 5.28 | 3.75 | 3.61 |
| BA10430 | ambi_miR_7058 | 5.84 | 6.07 | 5.52 | 6.19 | 5.45 | 5.73 | 5.56 | 5.98 | 5.68 | 5.73 |
| BA10243 | Hsa_miR_30e_5p | 5.78 | 6.50 | 6.32 | 6.78 | 6.50 | 6.93 | 6.21 | 6.38 | 6.15 | 6.69 |
| BA10291 | Hsa_miR_423 | 5.76 | 5.54 | 5.94 | 5.73 | 5.87 | 5.58 | 5.64 | 5.11 | 6.06 | 6.08 |
| BA10359 | Rno_miR_151_AS | 5.73 | 5.42 | 5.90 | 5.64 | 6.32 | 5.81 | 5.66 | 5.33 | 6.63 | 6.30 |
| BA10157 | Hsa_miR_15a | 5.72 | 5.73 | 5.57 | 5.60 | 5.65 | 5.72 | 4.60 | 4.85 | 5.31 | 5.97 |
| BA10261 | Hsa_miR_342 | 5.71 | 4.86 | 6.02 | 4.90 | 6.07 | 5.66 | 5.79 | 5.51 | 6.57 | 5.98 |
| BA10162 | Hsa_miR_18a | 5.69 | 5.44 | 6.23 | 5.56 | 6.49 | 6.11 | 5.10 | 4.82 | 6.52 | 6.17 |
| BA10306 | Hsa_miR_99a | 5.60 | 5.62 | 5.66 | 5.80 | 5.01 | 5.00 | 5.12 | 5.44 | 5.16 | 4.70 |
| BA10200 | Hsa_miR_210 | 5.55 | 6.46 | 5.45 | 7.05 | 4.44 | 6.79 | 6.49 | 6.82 | 5.34 | 7.38 |
| BA10267 | Hsa_miR_361 | 5.42 | 5.67 | 5.34 | 5.77 | 5.71 | 5.82 | 4.23 | 5.44 | 5.72 | 5.74 |
| BA10148 | Hsa_miR_148a | 5.36 | 6.03 | 4.67 | 5.80 | 4.58 | 4.76 | 3.77 | 4.68 | 4.66 | 5.10 |
| BA10289 | Hsa_miR_422a | 5.20 | 5.15 | 5.03 | 5.83 | 4.97 | 5.64 | 3.26 | 3.96 | 5.68 | 6.09 |
| BA10188 | Hsa_miR_19a | 5.09 | 5.48 | 5.10 | 5.18 | 5.01 | 4.74 | 3.09 | 3.08 | 5.43 | 4.91 |
| BA10170 | Hsa_miR_185 | 4.97 | 4.85 | 4.74 | 4.56 | 5.17 | 5.30 | 5.18 | 5.07 | 5.17 | 5.25 |
| BA10183 | Hsa_miR_197 | 4.77 | 3.48 | 3.92 | 3.23 | 4.18 | 3.65 | 4.01 | 3.57 | 4.08 | 4.13 |
| BA10126 | Hsa_miR_128a | 4.75 | 5.02 | 4.98 | 4.75 | 4.98 | 4.61 | 4.76 | 4.07 | 5.32 | 5.12 |
| BA10168 | Hsa_miR_183 | 4.70 | 4.85 | 3.57 | 4.58 | 4.51 | 4.44 | 4.32 | 4.14 | 4.28 | 4.49 |
| BA10292 | Hsa_miR_424 | 4.53 | 5.31 | 3.03 | 2.88 | 2.99 | 2.77 | 3.15 | 3.58 | 2.97 | 2.87 |
| BA10229 | Hsa_miR_29c | 4.50 | 5.72 | 4.66 | 5.05 | 4.65 | 5.16 | 3.15 | 4.59 | 4.66 | 4.96 |
| BA10153 | Hsa_miR_152 | 4.44 | 4.41 | 4.44 | 5.11 | 5.01 | 5.30 | 4.88 | 4.96 | 4.96 | 4.98 |
| BA10437 | Hsa_miR_494 | 4.40 | 6.53 | 5.71 | 5.96 | 5.55 | 5.95 | 5.87 | 7.12 | 5.92 | 5.16 |
| BA10448 | ambi_miR_7076 | 4.38 | 4.68 | 3.92 | 4.64 | 4.28 | 5.06 | 4.31 | 4.06 | 3.79 | 4.01 |
| BA10221 | Hsa_miR_26b | 4.36 | 6.12 | 4.55 | 5.76 | 4.25 | 5.04 | 2.48 | 4.54 | 5.02 | 5.21 |
| BA10156 | Hsa_miR_155 | 4.33 | 4.22 | 4.64 | 5.35 | 5.08 | 5.29 | 3.15 | 4.35 | 5.76 | 5.15 |
| BA10173 | Hsa_miR_188 | 4.28 | 2.65 | 3.66 | 2.80 | 3.38 | 3.51 | 4.46 | 3.70 | 3.15 | 2.94 |
| BA10432 | Hsa_miR_452 | 4.25 | 5.20 | 4.71 | 4.98 | 4.12 | 5.02 | 4.46 | 4.54 | 4.28 | 4.35 |
| BA10152 | Hsa_miR_151 | 4.23 | 4.74 | 4.84 | 4.82 | 5.11 | 5.25 | 5.11 | 4.99 | 5.28 | 5.43 |
| BA10224 | Hsa_miR_28 | 4.11 | 4.49 | 4.00 | 4.37 | 4.37 | 4.17 | 3.67 | 3.91 | 4.83 | 4.51 |
| BA10370 | Rno_miR_352 | 4.11 | 4.62 | 4.05 | 4.60 | 4.02 | 4.01 | 2.83 | 3.60 | 4.72 | 4.13 |
| BA10255 | Hsa_miR_331 | 4.01 | 4.21 | 4.33 | 4.09 | 5.51 | 4.47 | 4.39 | 4.29 | 4.43 | 4.56 |
| BA10123 | Hsa_miR_126 | 3.98 | 3.97 | 3.29 | 3.76 | 3.67 | 3.96 | 3.09 | 3.20 | 3.95 | 4.09 |
| BA10294 | Hsa_miR_429 | 3.93 | 5.25 | 3.97 | 5.04 | 4.39 | 4.82 | 3.31 | 4.19 | 4.48 | 4.78 |
| BA10304 | Hsa_miR_96 | 3.92 | 5.21 | 3.63 | 4.25 | 4.05 | 4.17 | 2.37 | 3.69 | 4.22 | 4.08 |
| BA10298 | Hsa_miR_7 | 3.87 | 3.36 | 3.97 | 3.30 | 4.13 | 3.25 | 1.99 | 2.22 | 5.21 | 3.72 |
| BA10248 | Hsa_miR_324_3p | 3.84 | 4.44 | 4.25 | 4.98 | 4.61 | 5.05 | 4.53 | 4.66 | 4.65 | 5.02 |
| BA10411 | ambi_miR_7039 | 3.83 | 3.25 | 3.75 | 3.32 | 4.04 | 3.73 | 4.27 | 3.89 | 3.45 | 4.11 |
| BA10305 | Hsa_miR_98 | 3.80 | 4.85 | 3.68 | 4.78 | 3.62 | 4.08 | 2.75 | 3.60 | 4.33 | 4.03 |
| BA10160 | Hsa_miR_17_3p | 3.79 | 3.50 | 4.23 | 3.54 | 4.04 | 3.68 | 4.41 | 3.55 | 3.82 | 3.48 |
| BA10208 | Hsa_miR_218 | 3.76 | 4.47 | 4.38 | 4.72 | 4.64 | 4.68 | 2.57 | 3.66 | 4.66 | 3.81 |
| BA10182 | Hsa_miR_196b | 3.76 | 4.26 | 3.50 | 3.81 | 3.95 | 3.85 | 1.34 | 2.31 | 4.33 | 3.87 |
| BA10149 | Hsa_miR_148b | 3.73 | 3.87 | 3.73 | 4.14 | 3.72 | 3.33 | 2.83 | 2.75 | 4.08 | 3.88 |
| BA10150 | Hsa_miR_149 | 3.70 | 4.06 | 3.73 | 4.78 | 3.93 | 4.57 | 4.55 | 4.22 | 4.53 | 5.25 |
| BA10268 | Hsa_miR_365 | 3.64 | 4.82 | 3.07 | 3.61 | 3.09 | 3.28 | 3.53 | 3.20 | 3.05 | 3.04 |
| BA10237 | Hsa_miR_30a_3p | 3.64 | 3.64 | 3.93 | 4.24 | 4.44 | 4.38 | 3.09 | 3.33 | 3.85 | 3.72 |
| BA10476 | Hsa_miR_500 | 3.59 | 3.55 | 3.58 | 3.90 | 3.87 | 4.36 | 4.04 | 3.63 | 3.69 | 3.67 |
| BA10458 | ambi_miR_7086 | 3.44 | 3.06 | 2.71 | 2.83 | 2.24 | 2.73 | 2.97 | 2.75 | 1.88 | 2.26 |
| BA10281 | Hsa_miR_378 | 3.43 | 3.21 | 3.70 | 3.33 | 3.45 | 3.57 | 3.44 | 3.03 | 3.63 | 4.06 |
| BA10259 | Hsa_miR_339 | 3.40 | 3.45 | 4.20 | 3.41 | 4.34 | 4.02 | 3.77 | 3.66 | 4.75 | 4.88 |
| BA10242 | Hsa_miR_30e_3p | 3.37 | 3.38 | 3.28 | 3.66 | 3.05 | 3.55 | 2.90 | 3.35 | 3.23 | 3.05 |
| BA10312 | mmu_miR_140_AS | 3.37 | 3.52 | 4.09 | 4.62 | 3.85 | 4.54 | 3.77 | 3.89 | 3.99 | 4.02 |
| BA10230 | Hsa_miR_301 | 3.33 | 3.43 | 3.52 | 3.41 | 3.94 | 3.77 | 3.40 | 2.62 | 4.28 | 4.06 |
| BA10137 | Hsa_miR_138 | 3.31 | 3.80 | 3.78 | 4.53 | 3.29 | 3.68 | 2.66 | 3.17 | 4.33 | 4.34 |
| BA10336 | mmu_miR_424 | 3.30 | 3.83 | 2.82 | 2.44 | 2.47 | 2.23 | 2.90 | 3.30 | 2.63 | 2.23 |
| BA10130 | Hsa_miR_132 | 3.28 | 4.43 | 3.58 | 4.58 | 4.29 | 4.75 | 2.97 | 3.99 | 4.22 | 5.25 |
| BA10477 | ambi_miR_7105 | 3.27 | 3.17 | 3.11 | 3.32 | 2.90 | 3.40 | 3.70 | 3.83 | 2.90 | 3.25 |
| BA10468 | Hsa_miR_505 | 3.26 | 2.89 | 3.49 | 3.23 | 3.85 | 3.43 | 2.97 | 2.40 | 3.40 | 3.26 |
| BA10276 | Hsa_miR_373_AS | 3.24 | 3.93 | 3.33 | 4.26 | 2.93 | 3.94 | 4.11 | 4.05 | 2.82 | 4.18 |
| BA10293 | Hsa_miR_425 | 3.24 | 2.72 | 3.37 | 3.11 | 3.29 | 3.07 | 3.26 | 2.80 | 3.09 | 3.20 |
| BA10178 | Hsa_miR_193a | 3.22 | 3.11 | 3.93 | 3.82 | 3.65 | 4.04 | 4.65 | 4.30 | 3.88 | 4.51 |
| BA10249 | Hsa_miR_324_5p | 3.20 | 2.62 | 2.99 | 3.06 | 2.69 | 2.86 | 3.56 | 3.50 | 2.98 | 3.30 |
| BA10118 | Hsa_miR_10b | 3.19 | 3.32 | 4.02 | 4.42 | 3.68 | 3.81 | 5.25 | 3.91 | 5.09 | 4.58 |
| BA10254 | Hsa_miR_330 | 3.17 | 2.64 | 2.87 | 2.78 | 3.48 | 2.90 | 3.03 | 1.40 | 3.92 | 3.30 |
| BA10402 | Hsa_miR_491 | 3.16 | 4.59 | 3.62 | 5.03 | 3.39 | 4.20 | 4.21 | 5.15 | 3.43 | 4.15 |
| BA10475 | ambi_miR_7103 | 3.15 | 2.47 | 3.34 | 2.64 | 3.23 | 2.77 | 2.83 | 2.14 | 3.93 | 2.69 |
| BA10180 | Hsa-miR_195 | 3.10 | 3.62 | 3.19 | 3.32 | 3.33 | 3.66 | 2.37 | 3.23 | 3.54 | 3.20 |
| BA10262 | Hsa_miR_345 | 3.09 | 3.24 | 2.94 | 3.26 | 2.98 | 3.04 | 2.57 | 2.39 | 3.27 | 3.74 |
| BA10333 | mmu_miR_298 | 3.05 | 3.68 | 4.53 | 4.31 | 4.26 | 4.39 | 4.86 | 4.56 | 4.69 | 3.94 |
| BA10364 | Rno_miR_336 | 3.03 | 2.92 | 3.06 | 2.89 | 2.53 | 2.59 | 3.03 | 3.20 | 2.33 | 2.79 |
| BA10315 | mmu_miR_17_3p | 3.01 | 2.76 | 3.57 | 3.09 | 3.25 | 3.04 | 3.40 | 2.90 | 3.48 | 3.07 |
| BA10428 | Hsa_miR_498 | 2.99 | 2.91 | 2.38 | 3.00 | 2.26 | 2.60 | 2.37 | 2.45 | 2.07 | 2.77 |
| BA10179 | Hsa_miR_194 | 2.99 | 4.94 | 3.43 | 4.68 | 4.35 | 5.37 | 3.83 | 4.74 | 4.48 | 5.85 |
| BA10456 | ambi_miR_7084 | 2.98 | 2.79 | 3.22 | 3.34 | 2.72 | 2.80 | 3.09 | 3.25 | 3.53 | 2.55 |
| BA10225 | Hsa_miR_296 | 2.96 | 2.40 | 2.69 | 2.59 | 2.47 | 2.35 | 4.53 | 3.20 | 2.38 | 2.32 |
| BA10447 | ambi_miR_7075 | 2.96 | 2.76 | 3.13 | 2.33 | 3.23 | 2.91 | 2.37 | 2.45 | 3.03 | 2.65 |
| BA10177 | Hsa_miR_192 | 2.96 | 5.10 | 2.99 | 4.84 | 3.80 | 5.28 | 3.09 | 4.40 | 4.07 | 5.46 |
| BA10313 | mmu_miR_151 | 2.94 | 3.94 | 3.78 | 3.98 | 4.07 | 4.13 | 2.97 | 3.26 | 4.43 | 4.06 |
| BA10450 | Hsa_miR_502 | 2.90 | 3.01 | 3.09 | 3.10 | 3.00 | 3.62 | 2.90 | 2.96 | 2.84 | 2.75 |
| BA10449 | Hsa_miR_501 | 2.87 | 2.29 | 2.47 | 2.78 | 3.27 | 2.81 | 2.57 | 2.69 | 3.05 | 2.65 |
| BA10429 | Hsa_miR_513 | 2.86 | 4.76 | 4.25 | 4.31 | 3.70 | 3.96 | 4.56 | 5.60 | 3.66 | 3.44 |
| BA10184 | Hsa_miR_198 | 2.86 | 3.78 | 4.54 | 4.47 | 4.12 | 4.49 | 4.59 | 4.67 | 4.80 | 3.56 |
| BA10265 | Hsa_miR_34b | 2.85 | 4.21 | 3.25 | 3.78 | 3.05 | 3.51 | 3.70 | 3.90 | 3.88 | 3.88 |
| BA10117 | Hsa_miR_10a | 2.76 | 2.65 | 2.62 | 2.69 | 2.11 | 2.30 | 1.51 | 2.44 | 4.31 | 3.43 |
| BA10314 | mmu_miR_155 | 2.76 | 3.07 | 4.91 | 5.99 | 5.21 | 6.17 | 2.57 | 5.29 | 6.24 | 5.48 |
| BA10165 | Hsa_miR_181c | 2.76 | 3.19 | 2.47 | 2.64 | 3.08 | 2.95 | 2.66 | 2.60 | 2.28 | 2.75 |
| BA10202 | Hsa_miR_212 | 2.76 | 2.61 | 2.24 | 2.69 | 2.37 | 2.62 | 1.84 | 2.53 | 2.55 | 2.73 |
| BA10343 | mmu_miR_345 | 2.76 | 2.83 | 3.50 | 3.74 | 3.07 | 3.40 | 3.15 | 3.09 | 3.56 | 3.21 |
| BA10270 | Hsa_miR_368 | 2.73 | 2.94 | 2.07 | 1.79 | 3.09 | 3.01 | 2.90 | 3.81 | 2.73 | 2.92 |
| BA10324 | mmu_miR_290 | 2.70 | 3.20 | 3.33 | 3.84 | 3.04 | 3.90 | 4.38 | 4.57 | 3.12 | 3.76 |
| BA10272 | Hsa_miR_370 | 2.67 | 2.70 | 3.95 | 3.46 | 3.43 | 3.39 | 4.63 | 4.51 | 3.85 | 3.30 |
| BA10436 | Hsa_miR_495 | 2.66 | 2.73 | 1.39 | 2.27 | 2.18 | 2.28 | 3.21 | 2.47 | 2.59 | 1.99 |
| BA10266 | Hsa_miR_34c | 2.59 | 2.76 | 3.83 | 3.84 | 3.70 | 4.21 | 3.67 | 3.91 | 4.47 | 3.87 |
| BA10316 | mmu_miR_192 | 2.59 | 4.96 | 3.14 | 4.70 | 3.38 | 5.03 | 2.97 | 4.02 | 3.93 | 5.20 |
| BA10171 | Hsa_miR_186 | 2.59 | 3.48 | 3.36 | 3.67 | 2.95 | 2.99 | 1.51 | 3.20 | 3.38 | 3.49 |
| BA10146 | Hsa_miR_146a | 2.56 | 3.45 | 3.12 | 3.64 | 3.33 | 4.06 | 3.26 | 3.85 | 3.20 | 3.31 |
| BA10139 | Hsa_miR_140 | 2.55 | 2.62 | 1.90 | 3.02 | 2.62 | 3.00 | 2.48 | 2.08 | 2.43 | 2.65 |
| BA10143 | Hsa_miR_143 | 2.51 | 1.91 | 4.51 | 4.53 | 4.58 | 4.84 | 4.55 | 4.49 | 5.20 | 4.37 |
| BA10453 | ambi_miR_7081 | 2.50 | 2.64 | 3.02 | 2.69 | 2.44 | 2.17 | 3.21 | 2.69 | 2.61 | 2.45 |
| BA10461 | ambi_miR_7089 | 2.47 | 2.59 | 2.09 | 2.54 | 1.92 | 2.28 | 1.84 | 2.57 | 2.48 | 2.60 |
| BA10174 | Hsa_miR_189 | 2.47 | 2.47 | 2.73 | 2.36 | 3.03 | 2.86 | 2.66 | 1.59 | 3.07 | 3.08 |
| BA10451 | ambi_miR_7079 | 2.40 | 1.82 | 1.29 | 1.99 | 2.34 | 1.81 | 2.75 | 1.51 | 2.95 | 3.02 |
| BA10452 | ambi_miR_7080 | 2.38 | 2.38 | 2.51 | 2.20 | 2.64 | 2.13 | 2.75 | 2.53 | 2.65 | 2.32 |
| BA10457 | ambi_miR_7085 | 2.38 | 2.44 | 2.81 | 2.59 | 2.39 | 2.25 | 2.75 | 2.79 | 2.63 | 1.88 |
| BA10172 | Hsa_miR_187 | 2.33 | 2.30 | 1.95 | 2.48 | 3.15 | 2.72 | 2.83 | 2.50 | 2.45 | 2.48 |
| BA10399 | ambi_miR_7027 | 2.33 | 3.57 | 2.62 | 3.25 | 2.15 | 3.11 | 2.48 | 2.88 | 2.57 | 2.73 |
| BA10311 | mmu_miR_129_3p | 2.33 | 2.49 | 3.03 | 2.42 | 2.47 | 2.17 | 2.25 | 2.57 | 2.57 | 2.03 |
| BA10256 | Hsa_miR_335 | 2.29 | 3.14 | 2.86 | 3.11 | 3.12 | 3.01 | 1.68 | 2.04 | 4.71 | 4.00 |
| BA10434 | ambi_miR_7062 | 2.23 | 2.24 | 2.75 | 2.05 | 2.99 | 2.63 | 2.83 | 2.52 | 2.78 | 3.11 |
| BA10362 | Rno_miR_327 | 2.22 | 3.01 | 3.29 | 3.29 | 3.30 | 3.44 | 3.36 | 3.33 | 3.79 | 2.73 |
| BA10463 | Hsa_miR_452_AS | 2.22 | 3.35 | 2.92 | 3.03 | 2.68 | 3.17 | 2.48 | 2.89 | 2.89 | 2.94 |
| BA10435 | Hsa_miR_432 | 2.22 | 2.26 | 3.23 | 2.56 | 3.13 | 2.99 | 3.53 | 3.17 | 3.89 | 2.73 |
| BA10169 | Hsa_miR_184 | 2.22 | 2.48 | 3.45 | 2.63 | 3.60 | 3.42 | 3.49 | 3.10 | 3.90 | 2.45 |
| BA10416 | Hsa_miR_518c_AS | 2.18 | 2.59 | 3.40 | 2.89 | 3.57 | 3.21 | 3.40 | 3.76 | 3.72 | 2.77 |
| BA10319 | mmu_miR_202 | 2.10 | 3.11 | 4.24 | 2.99 | 4.50 | 4.36 | 3.67 | 4.10 | 4.51 | 3.58 |
| BA10347 | mmu_miR_351 | 2.09 | 2.48 | 3.00 | 2.61 | 2.84 | 2.50 | 1.99 | 2.41 | 3.08 | 2.32 |
| BA10111 | Hsa_miR_101 | 2.05 | 2.70 | 2.22 | 2.49 | 1.99 | 2.29 | 1.68 | 2.53 | 2.75 | 2.45 |
| BA10309 | mmu_miR_101b | 2.05 | 2.00 | 1.92 | 2.55 | 2.43 | 2.46 | 2.25 | 2.40 | 2.89 | 2.16 |
| BA10346 | mmu_miR_350 | 2.03 | 2.18 | 2.99 | 3.37 | 3.00 | 3.04 | 3.03 | 2.78 | 3.46 | 2.60 |
| BA10423 | Hsa_miR_527 | 2.03 | 2.91 | 2.44 | 2.92 | 2.34 | 2.50 | 3.09 | 3.18 | 2.52 | 2.03 |
| BA10300 | Hsa_miR_9_AS | 1.94 | 2.02 | 1.87 | 1.89 | 2.01 | 1.96 | 2.66 | 2.28 | 3.03 | 2.90 |
| BA10119 | Hsa_miR_122a | 1.91 | 3.37 | 2.91 | 3.32 | 3.15 | 3.75 | 3.09 | 3.92 | 3.29 | 2.92 |
| BA10353 | mmu_miR_409 | 1.90 | 2.40 | 2.52 | 2.05 | 2.58 | 2.54 | 3.56 | 3.00 | 2.33 | 2.55 |
| BA10317 | mmu_miR_199b | 1.90 | 1.48 | 4.66 | 5.38 | 4.52 | 5.31 | 4.04 | 5.11 | 5.34 | 4.95 |
| BA10252 | Hsa_miR_328 | 1.90 | 2.05 | 2.42 | 2.55 | 2.76 | 2.27 | 2.90 | 2.65 | 2.61 | 2.45 |
| BA10405 | Hsa_miR_509 | 1.86 | 1.56 | 2.25 | 1.98 | 2.60 | 2.18 | 2.90 | 2.28 | 2.59 | 2.16 |
| BA10322 | mmu_miR_215 | 1.84 | 1.50 | 1.81 | 1.85 | 2.02 | 1.62 | 1.00 | 2.19 | 1.80 | 2.85 |
| BA10363 | Rno_miR_333 | 1.81 | 2.58 | 1.91 | 2.96 | 1.92 | 2.19 | 2.48 | 1.27 | 1.27 | 2.16 |
| BA10235 | Hsa_miR_302c_AS | 1.78 | 2.29 | 2.95 | 2.61 | 2.68 | 2.76 | 3.03 | 3.14 | 2.59 | 2.48 |
| BA10418 | Hsa_miR_526b | 1.72 | 1.55 | 2.83 | 2.04 | 2.53 | 2.52 | 2.83 | 2.70 | 2.65 | 1.56 |
| BA10332 | mmu_miR_297 | 1.66 | 2.81 | 1.57 | 3.83 | 1.46 | 2.74 | 1.00 | 3.00 | 1.80 | 2.16 |
| BA10465 | Hsa_miR_485_5p | 1.61 | 2.38 | 2.81 | 2.29 | 2.48 | 2.20 | 3.21 | 2.71 | 2.50 | 2.34 |
| BA10328 | mmu_miR_292_5p | 1.61 | 2.43 | 2.55 | 2.63 | 2.30 | 2.56 | 3.26 | 2.53 | 2.22 | 2.45 |
| BA10345 | mmu_miR_34b | 1.23 | 2.50 | 3.08 | 3.31 | 2.75 | 3.11 | 2.83 | 2.84 | 3.28 | 2.90 |
| BA10141 | Hsa_miR_142_3p | 1.22 | 1.66 | 2.49 | 1.37 | 2.81 | 2.41 | 2.66 | 2.71 | 2.85 | 2.51 |

### Example 3

### miRNAs Differentially Expressed Between NHK, HPV-Associated NHK, And HPV31-Associated cell line (CIN-612 9E)

Next, the inventors sought to determine if the presence of an HPV genome alters the miRNA expression profile of NHK. The inventors performed a global comparison of micro RNA expression data between NHK, HPV-associated NHK, and the HPV31-associated cell line (CIN-612 9E). Cells were grown and total RNA was prepared as described above in Example 1. MicroRNAs were purified and analyzed on microarrays as described above in Example 2.

HPV31-associated NHK monolayer cells vs NHK monolayer cells. Fifteen human miRNAs exhibited an average expression value that was significantly different between NHK monolayer cells (NHKM) and HPV31-associated NHK monolayer cells (NHK31M) (p-value (NHK31M-NHKM)<0.00220779). Among these, seven were up-regulated and six were down-regulated by at least 2-fold in NHK31M as compared to NHKM (|ΔH|(NHK31M vs NHKM)≧0.69) (Table 2). Among the up-regulated miRNAs, hsa-miR-199a, -199a_AS, and -214 were expressed at levels over 50-fold higher in NHK31M cells; hsa-miR-145 was up-regulated by over 15-fold, and hsa-miR-143 was expressed at least 5-fold higher in NHK31M compared to NHKM cells. Among the miRNAs down-regulated in NHK31M compared to NHKM cells, hsa-miR-503 was down-regulated by at least 10-fold, and hsa-miR-495 and -193b were down-regulated by at least 3-fold in NHK31M.

TABLE 2
miRNAs significantly differentially expressed between HPV31-associated NHK
monolayer cells (NHK31M) and NHK monolayer cells (NHKM).

| | | | p-value | | | p-value | ΔH | |
|---|---|---|---|---|---|---|---|---|
| | p-value | p-value | (Type * | Mean | Mean | (NHK31M − | (NHK31M − | |
| miRNA | (Type) | (Treatment) | Treatment) | (NHK M) | (NHK31M) | NHKM) | NHKM) | Fold Change |
| hsa-miR-199a | 5.94E−06 | 6.57E−01 | 6.47E−01 | 1.47 | 5.80 | 2.25E−05 | 4.33 | 76.0 |
| hsa-miR- | 4.17E−07 | 7.72E−02 | 2.54E−02 | 1.95 | 6.17 | 3.00E−06 | 4.22 | 68.2 |
| 199a_AS | | | | | | | | |
| hsa-miR-214 | 3.04E−05 | 2.22E−01 | 5.15E−01 | 1.75 | 5.83 | 5.96E−05 | 4.07 | 58.8 |
| hsa-miR-145 | 4.39E−05 | 5.01E−01 | 5.29E−01 | 1.95 | 4.75 | 2.99E−04 | 2.79 | 16.3 |
| hsa-miR-143 | 1.22E−04 | 2.36E−01 | 4.03E−01 | 2.51 | 4.51 | 9.48E−04 | 2.00 | 7.4 |
| hsa-miR-379 | 5.06E−03 | 3.90E−01 | 4.37E−02 | 1.64 | 3.11 | 1.99E−03 | 1.47 | 4.4 |
| hsa-miR-19b | 2.36E−03 | 4.41E−01 | 9.77E−02 | 7.10 | 7.83 | 1.94E−03 | 0.73 | 2.1 |
| hsa-miR-151 | 1.47E−04 | 5.04E−02 | 4.54E−02 | 4.23 | 4.84 | 8.36E−04 | 0.61 | 1.8 |
| hsa-miR-30a-5p | 6.75E−06 | 2.48E−06 | 2.42E−02 | 6.90 | 7.19 | 1.19E−03 | 0.29 | 1.3 |
| hsa-miR-200a | 3.03E−05 | 3.75E−06 | 5.47E−05 | 6.42 | 5.65 | 3.58E−06 | −0.77 | 2.2 |
| hsa-miR-197 | 1.12E−02 | 1.75E−04 | 1.35E−02 | 4.77 | 3.92 | 1.00E−03 | −0.85 | 2.3 |
| hsa-miR-200b | 1.94E−04 | 1.18E−04 | 1.30E−02 | 7.74 | 6.80 | 8.65E−05 | −0.94 | 2.6 |
| hsa-miR-193b | 8.00E−03 | 6.56E−05 | 2.42E−02 | 7.08 | 5.84 | 9.76E−04 | −1.24 | 3.4 |
| hsa-miR-495 | 2.26E−04 | 4.70E−01 | 1.29E−03 | 2.66 | 1.39 | 4.85E−05 | −1.27 | 3.6 |
| hsa-miR-503 | 3.25E−04 | 9.22E−05 | 1.91E−01 | 7.37 | 5.00 | 1.50E−04 | −2.37 | 10.7 |

HPV18-associated NHK monolayer cells vs NHK monolayer cells. Fourteen human miRNAs exhibited an average expression value that was significantly different between NHKM cells and HPV18-associated NHK monolayer cells (NHK18M) (p-value (NHK18M-NHKM)<0.00220779). Among these, eleven were up-regulated and three were down-regulated by at least 2-fold in NHK18M as compared to NHKM (Table 3). Three miRNAs (hsa-miR-199, -199a, and -214) were expressed at levels over 50-fold higher in NHK18M; hsa-miR-145 was up-regulated by more than 20-fold, and hsa-miR-143 was up-regulated by over 5-fold. Among the miRNAs down-regulated in NHK18M, hsa-miR-503 was down-regulated by over 5-fold (ΔH(NHK18M-NHKM)<−1.6).

TABLE 3
MicroRNAs significantly differentially expressed between HPV18-associated NHK
monolayer cells (NHK18M) and NHK monolayer cells (NHKM).

| | | | p-value | | | p-value | ΔH | |
|---|---|---|---|---|---|---|---|---|
| | p-value | p-value | (Type * | Mean | Mean | (NHK18M − | (NHK18M − | |
| miRNA | (Type) | (Treatment) | Treatment) | (NHK M) | (NHK18M) | NHKM) | NHKM) | Fold Change |
| hsa-miR-199a | 5.94E−06 | 6.57E−01 | 6.47E−01 | 1.47 | 5.71 | 2.57E−05 | 4.25 | 69.8 |
| hsa-miR-199a_AS | 4.17E−07 | 7.72E−02 | 2.54E−02 | 1.95 | 6.07 | 3.58E−06 | 4.11 | 61.1 |
| hsa-miR-214 | 3.04E−05 | 2.22E−01 | 5.15E−01 | 1.75 | 5.73 | 7.03E−05 | 3.97 | 53.0 |
| hsa-miR-145 | 4.39E−05 | 5.01E−01 | 5.29E−01 | 1.95 | 4.97 | 1.87E−04 | 3.01 | 20.3 |
| hsa-miR-143 | 1.22E−04 | 2.36E−01 | 4.03E−01 | 2.51 | 4.58 | 7.75E−04 | 2.07 | 7.9 |
| hsa-miR-142-3p | 2.40E−03 | 1.05E−01 | 5.84E−02 | 1.22 | 2.81 | 9.27E−04 | 1.60 | 4.9 |
| hsa-miR-132 | 5.94E−04 | 2.35E−05 | 1.90E−01 | 3.28 | 4.29 | 9.65E−04 | 1.01 | 2.7 |
| hsa-miR-151 | 1.47E−04 | 5.04E−02 | 4.54E−02 | 4.23 | 5.11 | 8.47E−05 | 0.88 | 2.4 |
| hsa-miR-19b | 2.36E−03 | 4.41E−01 | 9.77E−02 | 7.10 | 7.83 | 1.95E−03 | 0.73 | 2.1 |
| hsa-miR-30e-5p | 4.31E−03 | 2.57E−04 | 2.38E−01 | 5.78 | 6.50 | 1.07E−03 | 0.72 | 2.1 |
| hsa-miR-30a-5p | 6.75E−06 | 2.48E−06 | 2.42E−02 | 6.90 | 7.59 | 4.62E−06 | 0.69 | 2.0 |
| hsa-miR-375 | 1.21E−03 | 9.30E−01 | 1.25E−03 | 2.87 | 2.03 | 7.35E−04 | −0.85 | 2.3 |
| ambi-miR-7086 | 6.04E−03 | 5.49E−01 | 1.98E−01 | 3.44 | 2.24 | 1.73E−03 | −1.21 | 3.3 |
| hsa-miR-503 | 3.25E−04 | 9.22E−05 | 1.91E−01 | 7.37 | 5.53 | 7.06E−04 | −1.84 | 6.3 |

HPV11-associated NHK monolayer cells vs NHK monolayer cells. Thirty-one human miRNAs exhibited an average expression value that was significantly different between NHK monolayer cells and HPV11-associated NHK monolayer cells (NHK11M) (p-value (NHK11M-NHKM)<0.00441558). Among these, 11 were up-regulated and 18 were down-regulated by at least 2-fold in NHK11M as compared to NHKM (|ΔH|(NHK11M vs NHKM)≧0.69) (Table 4). Two miRNAs (hsa-miR-199a and -214) were up-regulated by over 50-fold, two (hsa-miR-199a_AS and -145) were up-regulated by 10 to 50-fold, and two (hsa-miR-143 and -379) were up-regulated by 5 to 10-fold in NHK11M. Among the miRNAs down-regulated in NHK11M cells, hsa-let-7g was down-regulated by over 20-fold, hsa-miR-196b was down-regulated by 10- to 20-fold, and 7 miRNAs (hsa-miR-20a, -19a, -422a, -25, -7c, -7a, and -15b) were down-regulated by 5- to 10-fold.

TABLE 4
MicroRNAs significantly differentially expressed between HPV11-associated NHK
monolayer cells (NHK11M) and NHK monolayer cells (NHKM).

| | | | p-value | | | p-value | ΔH | |
|---|---|---|---|---|---|---|---|---|
| | p-value | p-value | (Type * | Mean | Mean | (NHK11M − | (NHK11M − | |
| miRNA | (Type) | (Treatment) | Treatment) | (NHK M) | (NHK11M) | NHKM) | NHKM) | Fold Change |
| hsa-miR-199a | 5.94E−06 | 6.57E−01 | 6.47E−01 | 1.47 | 5.68 | 1.00E−04 | 4.22 | 67.7 |
| hsa-miR-214 | 3.04E−05 | 2.22E−01 | 5.15E−01 | 1.75 | 5.87 | 2.01E−04 | 4.12 | 61.5 |
| hsa-miR- | 4.17E−07 | 7.72E−02 | 2.54E−02 | 1.95 | 5.30 | 5.36E−05 | 3.35 | 28.5 |
| 199a_AS | | | | | | | | |
| hsa-miR-145 | 4.39E−05 | 5.01E−01 | 5.29E−01 | 1.95 | 4.90 | 7.28E−04 | 2.95 | 19.1 |
| hsa-miR-143 | 1.22E−04 | 2.36E−01 | 4.03E−01 | 2.51 | 4.55 | 2.70E−03 | 2.04 | 7.7 |
| hsa-miR-379 | 5.06E−03 | 3.90E−01 | 4.37E−02 | 1.64 | 3.44 | 1.97E−03 | 1.81 | 6.1 |
| hsa-miR-203 | 5.34E−02 | 2.52E−07 | 1.15E−02 | 7.30 | 8.60 | 3.17E−03 | 1.30 | 3.7 |
| hsa-miR-22 | 2.00E−03 | 9.48E−04 | 2.00E−01 | 7.13 | 8.21 | 8.23E−04 | 1.08 | 2.9 |
| hsa-miR-34a | 3.42E−03 | 2.49E−03 | 2.81E−01 | 6.22 | 7.27 | 1.58E−03 | 1.06 | 2.9 |
| hsa-miR-151 | 1.47E−04 | 5.04E−02 | 4.54E−02 | 4.23 | 5.11 | 3.06E−04 | 0.88 | 2.4 |
| hsa-miR-27b | 6.03E−02 | 6.16E−01 | 2.02E−02 | 6.96 | 7.77 | 4.11E−03 | 0.81 | 2.2 |
| hsa-miR-27a | 3.10E−02 | 1.82E−02 | 1.72E−02 | 8.29 | 8.93 | 3.91E−03 | 0.65 | 1.9 |
| hsa-miR-30d | 6.33E−05 | 2.84E−07 | 7.65E−03 | 6.69 | 6.42 | 2.87E−03 | −0.27 | 1.3 |
| hsa-miR-200b | 1.94E−04 | 1.18E−04 | 1.30E−02 | 7.74 | 6.96 | 9.14E−04 | −0.78 | 2.2 |
| hsa-miR-29a | 1.21E−04 | 3.47E−04 | 3.39E−02 | 8.78 | 7.98 | 1.09E−04 | −0.81 | 2.2 |
| hsa-miR-92 | 2.46E−03 | 3.18E−01 | 7.70E−02 | 7.81 | 6.94 | 2.19E−03 | −0.87 | 2.4 |
| hsa-miR-16 | 6.47E−04 | 7.69E−02 | 2.18E−01 | 8.92 | 7.90 | 1.06E−03 | −1.02 | 2.8 |
| hsa-miR-32 | 4.82E−04 | 7.91E−01 | 1.04E−01 | 2.14 | 1.00 | 3.27E−03 | −1.14 | 3.1 |
| hsa-let-7e | 7.90E−03 | 1.21E−03 | 2.26E−01 | 7.02 | 5.86 | 4.23E−03 | −1.15 | 3.2 |
| hsa-let-7i | 2.21E−04 | 5.46E−02 | 7.97E−03 | 6.68 | 5.40 | 2.39E−04 | −1.28 | 3.6 |
| hsa-let-7d | 2.04E−03 | 3.84E−02 | 7.50E−02 | 8.14 | 6.79 | 7.97E−04 | −1.35 | 3.9 |
| hsa-miR-96 | 2.78E−04 | 3.38E−04 | 7.62E−03 | 3.92 | 2.37 | 3.30E−04 | −1.55 | 4.7 |
| hsa-miR-15b | 3.57E−04 | 1.29E−02 | 1.91E−01 | 7.80 | 6.15 | 4.42E−04 | −1.65 | 5.2 |
| hsa-let-7a | 2.58E−04 | 8.01E−03 | 2.63E−03 | 9.39 | 7.69 | 2.15E−05 | −1.70 | 5.5 |
| hsa-let-7c | 2.12E−03 | 2.75E−01 | 2.28E−02 | 8.88 | 7.12 | 2.03E−04 | −1.77 | 5.8 |
| hsa-miR-25 | 6.37E−04 | 3.55E−03 | 2.13E−01 | 6.16 | 4.27 | 5.75E−04 | −1.89 | 6.6 |
| hsa-miR-422a | 2.45E−04 | 7.87E−03 | 2.76E−01 | 5.20 | 3.26 | 6.00E−04 | −1.95 | 7.0 |
| hsa-miR-19a | 1.40E−03 | 7.37E−01 | 6.79E−01 | 5.09 | 3.09 | 4.30E−03 | −2.00 | 7.4 |
| hsa-miR-20a | 1.54E−03 | 4.82E−01 | 9.71E−02 | 6.87 | 4.83 | 6.88E−04 | −2.04 | 7.7 |
| hsa-miR-196b | 5.90E−05 | 9.15E−02 | 8.27E−02 | 3.76 | 1.34 | 8.75E−05 | −2.42 | 11.3 |
| hsa-let-7g | 1.90E−03 | 4.43E−03 | 1.93E−02 | 6.87 | 3.86 | 2.09E−04 | −3.02 | 20.4 |

HPV31-associated CIN-612 9E monolayer cells vs NHK monolayer cells. Thirteen human miRNAs were expressed with a significant differential expression between the CIN-derived cell line CIN-612-9EM and NHK monolayer cells (NHKM) (p-value (CIN-612-9EM-NHKM)<0.00181818), including seven up-regulated and four down-regulated miRNAs in CIN-612-9EM compared to NHKM cells, by at least 2-fold (|ΔH|(CIN-612-9EM vs NHKM)≧0.69) (Table 5). Among the miRNAs up-regulated in CIN-612-9EM, 3 miRNAs (hsa-miR-199aAS, -199a, and -214) were overexpressed by more than 100-fold; two miRNAs (hsa-miR-145 and -143) were overexpressed by more than 10-fold; and one miRNA (hsa-miR-379) was up-regulated by more than 5-fold. Among the miRNAs that were down-regulated in CIN-612-9EM versus NHKM, hsa-miR-503 was expressed with a level at least 10-fold lower, and hsa-miR-196a was down-regulated by at least 5-fold (Table 5).

TABLE 5
miRNAs significantly differentially expressed between HPV31-associated CIN-612 9E
monolayer cells (CIN-612-9EM) and NHK monolayer cells (NHKM).

| | | | | | | p-value | ΔH | |
|---|---|---|---|---|---|---|---|---|
| | p-value | p-value | p-value | Mean | Mean | (CIN-612_-9EM − | (CIN-612-9EM − | Fold |
| miRNA | (Type) | (Treatment) | (Type * Treatment) | (NHK M) | (CIN-612-9EM) | NHK-M) | NHKM) | Change |
| hsa-miR-199a_AS | 4.17E−07 | 7.72E−02 | 2.54E−02 | 1.95 | 7.09 | 3.14E−06 | 5.14 | 170.1 |
| hsa-miR-199a | 5.94E−06 | 6.57E−01 | 6.47E−01 | 1.47 | 6.37 | 3.80E−05 | 4.90 | 134.0 |
| hsa-miR-214 | 3.04E−05 | 2.22E−01 | 5.15E−01 | 1.75 | 6.64 | 6.87E−05 | 4.88 | 131.8 |
| hsa-miR-145 | 4.39E−05 | 5.01E−01 | 5.29E−01 | 1.95 | 5.53 | 2.27E−04 | 3.58 | 35.8 |
| hsa-miR-143 | 1.22E−04 | 2.36E−01 | 4.03E−01 | 2.51 | 5.20 | 5.48E−04 | 2.69 | 14.7 |
| hsa-miR-379 | 5.06E−03 | 3.90E−01 | 4.37E−02 | 1.64 | 3.80 | 7.00E−04 | 2.16 | 8.7 |
| hsa-miR-151 | 1.47E−04 | 5.04E−02 | 4.54E−02 | 4.23 | 5.28 | 9.94E−05 | 1.05 | 2.9 |
| hsa-miR-30a-5p | 6.75E−06 | 2.48E−06 | 2.42E−02 | 6.90 | 7.28 | 7.18E−04 | 0.38 | 1.5 |
| hsa-miR-200a | 3.03E−05 | 3.75E−06 | 5.47E−05 | 6.42 | 5.89 | 1.46E−04 | −0.54 | 1.7 |
| hsa-miR-375 | 1.21E−03 | 9.30E−01 | 1.25E−03 | 2.87 | 1.42 | 9.41E−05 | −1.45 | 4.3 |
| ambi-miR-7086 | 6.04E−03 | 5.49E−01 | 1.98E−01 | 3.44 | 1.88 | 1.24E−03 | −1.57 | 4.8 |
| hsa-miR-196a | 3.36E−04 | 3.02E−01 | 4.45E−01 | 5.86 | 3.75 | 4.25E−04 | −2.10 | 8.2 |
| hsa-miR-503 | 3.25E−04 | 9.22E−05 | 1.91E−01 | 7.37 | 5.01 | 5.36E−04 | −2.36 | 10.6 |

Common and different miRNAs differentially expressed between HPV-associated cell lines and HPV-negative NHK cells grown as monolayers. Further analysis of the array data was performed to identify the miRNAs whose expression was commonly altered in NHK11M, NHK18M, NHK31M, and CIN-612-9EM, compared to NHKM cells. In particular, as the CIN-612 9E cell line is associated with HPV31, it was of interest to directly compare the miRNAs differentially expressed between CIN-612-9EM and NHKM with the miRNAs differentially expressed between NHK31M and NHKM. The comparison revealed that eight miRNAs, (hsa-miR-199a_AS, -199a, -214, -145, -143, -379, -151, and -30a_{—}5p) were over-expressed in both HPV31-associated cell lines (NHK31M and CIN-612 9EM) compared to their expression in primary keratinocytes (NHKM) and that two miRNAs (hsa-miR-503 and -200a) were down-regulated in NHK31M and CIN-612-9EM compared to NHKM. When the comparison was extended to include HPV18- and HPV11-associated NHK, the analysis showed that hsa-miR-199a, -199a-AS, -145, -214, -143, and -151, were up-regulated in all HPV-associated NHK compared to NHKM cells, strongly suggesting that up-regulation of these miRNAs is linked to the presence of an HPV genome in the cells. Also, hsa-miR-503 was down-regulated between NHK18M and NHKM cells but not between NHK11M and NHKM. As HPV11 is usually associated with benign mucosal lesions, in contrast to HPV31 and HPV18, these data suggest that differential expression of hsa-miR-503 between an HPV-associated NHK and NHK is linked to the oncogenic potential of the HPV type. In addition, a total of nine miRNAs (hsa-let7a, -let7d, -let7i, -let7g, and hsa-miR-96, -25, -422, -20a, -and -196b) were found up-regulated in all the HPV18- and HPV31-associated cell lines (NHK18M, NHK31M and CIN-612-9EM) compared to HPV11-associated NHK (NHK11M). The differential expression of these miRNAs may be linked to the higher oncogenic potential of HPV18 and HPV31 compared to HPV11. Importantly, no miRNA was found differentially expressed between NHK31M and CIN-612-9EM or between NHK18M and CIN-612-9EM, suggesting that the effect of the presence of an HPV in primary keratinocytes is conserved among HPV types of similar oncogenicity. Two miRNAs (hsa-miR-200a and -30a-5p) were found significantly up-regulated in NHK18M compared to NHK31M, but the fold-change observed is less than 2-fold.

### Example 4

### miRNA Expression Changes Induced by Cell Differentiation in NHK, HPV-Associated NHK, and HPV31-Associated Cell Line (CIN-612 9E)

miRNAs have been shown to have important roles in many cellular processes, including cell differentiation. Because HPV survival is dependent on the expression of the keratinocyte differentiation program, the inventors investigated whether the presence of HPV affects the normal changes induced by cellular differentiation in the miRNA expression profile of NHK. Therefore, the investigators identified the miRNA expression profile of primary keratinocytes before and after induction of differentiation and compared those with the microRNA expression profiles of HPV-associated NHK before and after differentiation.

Prior to induction of cellular differentiation, monolayer cell cultures were transferred in semisolid medium containing methylcellulose. Culture under these conditions induces keratinocyte differentiation and, as a consequence, stimulates viral late functions such as genomic amplification and late transcription. Cell extraction and total RNA isolation were performed at 24 hr and 48 hr after induction of differentiation. Cells were grown, and total RNA was prepared as described above in Example 1. MicroRNAs were purified and analyzed on microarrays as described above in Example 2. For each cell type, miRNA expression profiles of differentiated cells were compared with those of cells before differentiation (monolayer cells, M). However, as no significant difference was noted between 24 hr and 48 hr of differentiation, and since the cellular response to the differentiation signal is not yet complete at 24 hr, we have only reported the miRNAs differentially expressed between undifferentiated cells (cells grown in monolayers, M) and cells that were induced to differentiate for 48 hr (48) in methylcellulose.

NHK before differentiation (NHKM) vs NHK 48 hr after differentiation (NHK48). A total of 22 human miRNAs were expressed with a significant differential expression in NHK after 48 hr of treatment with methylcellulose (p-value (NHK48-NHKM)<0.00298701) (Table 6). Among these, nine miRNAs were up-regulated and five miRNAs were down-regulated with at least a 2-fold change (|ΔH|(NHK48 vs NHKM)≧0.69), as a result of induction of cell differentiation. Among the up-regulated miRNAs, hsa-miR-203 was up-regulated by more than 20-fold, and hsa-miR-192 and -194 were up-regulated between 5- and 10-fold. Among the five down-regulated miRNAs, hsa-miR-503 was down-regulated by more than 5-fold at 48 hr after induction of differentiation (Table 6). Interestingly, none of the miRNAs significantly differentially expressed between HPV-associated cell lines and NHK, i.e. hsa-miR-199a, -199a-AS, -214, -145, and 143, was shown to have its level of expression significantly affected by the induction of differentiation in NHK cells.

TABLE 6
miRNAs differentially expressed between NHK before differentiation and 48 hr after
differentiation. NHKM, NHK monolayer cells before differentiation; NHK48, NHK 48 hr after
differentiation. Negative ΔH values indicate down-regulation in NHK48.

| | | | p-value | | | p-value | | |
|---|---|---|---|---|---|---|---|---|
| | p-value | p-value | (Type * | Mean | Mean | (NHK48 − | ΔH (NHK48 − | Fold |
| miRNA | (Type) | (Treatment) | Treatment) | (NHKM) | (NHK48) | NHKM) | NHKM) | Change |
| hsa-miR-203 | 5.34E−02 | 2.52E−07 | 1.15E−02 | 7.30 | 10.62 | 2.56E−06 | 3.32 | 27.6 |
| hsa-miR-192 | 5.37E−02 | 4.62E−05 | 5.56E−01 | 2.96 | 5.10 | 5.68E−04 | 2.14 | 8.5 |
| hsa-miR-194 | 3.32E−02 | 4.21E−04 | 5.06E−01 | 2.99 | 4.94 | 1.99E−03 | 1.95 | 7.1 |
| hsa-miR-491 | 1.50E−02 | 9.72E−05 | 3.37E−01 | 3.16 | 4.59 | 9.53E−04 | 1.43 | 4.2 |
| hsa-miR-224 | 9.29E−02 | 1.68E−04 | 4.01E−01 | 6.34 | 7.63 | 2.08E−03 | 1.29 | 3.6 |
| hsa-miR-96 | 2.78E−04 | 3.38E−04 | 7.62E−03 | 3.92 | 5.21 | 2.88E−04 | 1.29 | 3.6 |
| ambi-miR-7027 | 1.40E−01 | 2.23E−04 | 6.34E−02 | 2.33 | 3.57 | 3.47E−04 | 1.24 | 3.5 |
| hsa-miR-132 | 5.94E−04 | 2.35E−05 | 1.90E−01 | 3.28 | 4.43 | 4.45E−04 | 1.15 | 3.2 |
| hsa-miR-30e-5p | 4.31E−03 | 2.57E−04 | 2.38E−01 | 5.78 | 6.50 | 1.05E−03 | 0.72 | 2.1 |
| hsa-miR-27a | 3.10E−02 | 1.82E−02 | 1.72E−02 | 8.29 | 8.95 | 1.09E−03 | 0.66 | 1.9 |
| hsa-miR-151 | 1.47E−04 | 5.04E−02 | 4.54E−02 | 4.23 | 4.74 | 2.32E−03 | 0.51 | 1.7 |
| hsa-miR-26a | 8.29E−04 | 5.78E−04 | 5.16E−01 | 7.75 | 8.24 | 2.28E−03 | 0.49 | 1.6 |
| hsa-miR-30a-5p | 6.75E−06 | 2.48E−06 | 2.42E−02 | 6.90 | 7.39 | 4.61E−05 | 0.49 | 1.6 |
| hsa-miR-200a | 3.03E−05 | 3.75E−06 | 5.47E−05 | 6.42 | 6.87 | 1.22E−04 | 0.45 | 1.6 |
| hsa-miR-29a | 1.21E−04 | 3.47E−04 | 3.39E−02 | 8.78 | 9.18 | 2.19E−03 | 0.40 | 1.5 |
| hsa-miR-30d | 6.33E−05 | 2.84E−07 | 7.65E−03 | 6.69 | 7.02 | 3.00E−04 | 0.33 | 1.4 |
| hsa-miR-30c | 4.71E−05 | 8.63E−06 | 2.56E−02 | 6.85 | 7.16 | 1.93E−03 | 0.32 | 1.4 |
| hsa-miR-130a | 6.47E−03 | 7.49E−04 | 3.88E−01 | 7.35 | 6.51 | 2.55E−03 | −0.85 | 2.3 |
| hsa-miR-197 | 1.12E−02 | 1.75E−04 | 1.35E−02 | 4.77 | 3.48 | 7.95E−05 | −1.29 | 3.6 |
| hsa-miR-181b | 5.01E−02 | 7.25E−03 | 1.88E−01 | 7.53 | 6.21 | 2.96E−03 | −1.32 | 3.7 |
| hsa-miR-130b | 2.08E−02 | 2.95E−03 | 4.62E−02 | 6.06 | 4.70 | 7.78E−04 | −1.36 | 3.9 |
| hsa-miR-503 | 3.25E−04 | 9.22E−05 | 1.91E−01 | 7.37 | 5.29 | 3.37E−04 | −2.08 | 8.0 |

HPV31-associated NHK before differentiation (NHK31M) vs HPV31-associated NHK 48 hr after differentiation (NHK3148). Thirteen miRNAs were expressed with a significant differential expression after 48 hr of induction of cellular differentiation with methylcellulose (p-value (NHK3148-NHK31M)<0.00181818), including eleven up-regulated miRNAs in NHK3148 compared to NHK31M by more than 2-fold (ΔH(NHK3148 vs NHK31M)≧0.69) (Table 7). Among these, four miRNAs (hsa-miR-203, -30b, -192, and -193b) were up-regulated between 5-and 10-fold in NHK3148. No miRNA was observed to be down-regulated after induction of differentiation.

TABLE 7
miRNAs differentially expressed between HPV31-associated-NHK before
differentiation and 48 hr after differentiation. NHK31M, HPV31-associated NHK monolayer
cells before differentiation; NHK3148, HPV31-associated NHK 48 hr after differentiation.

| | | | p-value | | | p-value | ΔH | |
|---|---|---|---|---|---|---|---|---|
| | p-value | p-value | (Type * | Mean | Mean | (NHK3148 − | (NHK3148 − | Fold |
| miRNA | (Type) | (Treatment) | Treatment) | (NHK31M) | (NHK3148) | NHK31M) | NHK31M) | Change |
| hsa-miR-203 | 5.34E−02 | 2.52E−07 | 1.15E−02 | 7.85 | 10.16 | 2.86E−05 | 2.31 | 10.1 |
| hsa-miR-30b | 1.14E−02 | 1.16E−04 | 1.95E−01 | 5.42 | 7.38 | 4.26E−04 | 1.96 | 7.1 |
| hsa-miR-192 | 5.37E−02 | 4.62E−05 | 5.56E−01 | 2.99 | 4.84 | 1.35E−03 | 1.85 | 6.3 |
| hsa-miR-193b | 8.00E−03 | 6.56E−05 | 2.42E−02 | 5.84 | 7.46 | 1.94E−04 | 1.62 | 5.0 |
| hsa-miR-210 | 2.68E−02 | 3.58E−05 | 2.36E−02 | 5.45 | 7.05 | 1.25E−03 | 1.60 | 5.0 |
| hsa-miR-491 | 1.50E−02 | 9.72E−05 | 3.37E−01 | 3.62 | 5.03 | 1.06E−03 | 1.41 | 4.1 |
| hsa-miR-132 | 5.94E−04 | 2.35E−05 | 1.90E−01 | 3.58 | 4.58 | 9.92E−04 | 1.00 | 2.7 |
| hsa-miR-200b | 1.94E−04 | 1.18E−04 | 1.30E−02 | 6.80 | 7.80 | 5.83E−05 | 1.00 | 2.7 |
| hsa-miR-200a | 3.03E−05 | 3.75E−06 | 5.47E−05 | 5.65 | 6.59 | 9.40E−07 | 0.94 | 2.5 |
| hsa-miR-495 | 2.26E−04 | 4.70E−01 | 1.29E−03 | 1.39 | 2.27 | 4.88E−04 | 0.88 | 2.4 |
| hsa-miR-30d | 6.33E−05 | 2.84E−07 | 7.65E−03 | 6.56 | 7.27 | 1.95E−06 | 0.71 | 2.0 |
| hsa-miR-30c | 4.71E−05 | 8.63E−06 | 2.56E−02 | 6.86 | 7.49 | 2.58E−05 | 0.64 | 1.9 |
| hsa-miR-30a-5p | 6.75E−06 | 2.48E−06 | 2.42E−02 | 7.19 | 7.75 | 1.80E−05 | 0.56 | 1.8 |

HPV18-associated NHK before differentiation (NHK18M) vs HPV18-associated NHK 48 hr after differentiation (NHK1848). Four miRNAs were up-regulated with a significant differential expression in NHK1848 versus NHK18M after 48 hr of induction of differentiation of the cells with methylcellulose (p-value (NHK1848-NHKM)<0.00051948) by more than 2-fold (|ΔH|(NHK1848 vs NHK18M)≧0.69) (Table 8). Among these, two miRNAs (hsa-miR-210 and -203), were up-regulated between 5- and 10-fold in NHK1848 (Table 8). No miRNA was observed to be down regulated after induction of differentiation.

TABLE 8
miRNAs differentially expressed between HPV18-associated-NHK before
differentiation and 48 hr after differentiation. NHK18M, HPV18-associated NHK monolayer
cells before differentiation; NHK1848, HPV18-associated NHK 48 hr after differentiation.
Negative ΔH values indicate down-regulation in NHK1848.

| | | | p-value | | | p-value | ΔH | |
|---|---|---|---|---|---|---|---|---|
| | p-value | p-value | (Type * | Mean | Mean | (NHK1848 − | (NHK1848 − | Fold |
| miRNA | (Type) | (Treatment) | Treatment) | (NHK18M) | (NHK1848) | NHK18M) | NHK18M) | Change |
| hsa-miR-210 | 2.68E−02 | 3.58E−05 | 2.36E−02 | 4.44 | 6.79 | 1.20E−04 | 2.35 | 10.5 |
| hsa-miR-203 | 5.34E−02 | 2.52E−07 | 1.15E−02 | 8.02 | 9.95 | 9.33E−05 | 1.93 | 6.9 |
| hsa-miR-30d | 6.33E−05 | 2.84E−07 | 7.65E−03 | 6.77 | 7.24 | 2.95E−05 | 0.47 | 1.6 |
| hsa-miR-30c | 4.71E−05 | 8.63E−06 | 2.56E−02 | 7.09 | 7.51 | 3.66E−04 | 0.42 | 1.5 |

HPV11-associated NHK before differentiation (NHK11M) vs HPV11-associated NHK 48 hr after differentiation (NHK1148). Twelve human miRNAs were expressed with a significant differential expression in HPV11-associated NHK after induction of differentiation for 48 hr, using methylcellulose (p-value (NHK1148-NHK11M)<0.00168831) (Table 9), including six up-regulated and two down-regulated in HPV11-differentiated cells by at least 2-fold (Table 9). Among these, only hsa-let-7g was overexpressed by more than 5-fold and only hsa-miR-330 was down-regulated by more than 5-fold (Table 9).

TABLE 9
miRNAs differentially expressed between HPV11-associated-NHK before
differentiation and 48 hr after differentiation. NHK11M, HPV11-associated NHK monolayer
cells before differentiation; NHK1148, HPV11-associated NHK 48 hr after differentiation.
Negative ΔH values indicate down-regulation in NHK1148.

| | | | p-value | | | p-value | ΔH | |
|---|---|---|---|---|---|---|---|---|
| | p-value | p-value | (Type * | Mean | Mean | (NHK1148 − | (NHK1148 − | Fold |
| miRNA | (Type) | (Treatment) | Treatment) | (NHK1148) | (NHK11M) | NHK11M) | NHK11M) | Change |
| hsa-let-7g | 1.90E−03 | 4.43E−03 | 1.93E−02 | 6.40 | 3.86 | 5.90E−04 | 2.54 | 12.7 |
| hsa-miR-203 | 5.34E−02 | 2.52E−07 | 1.15E−02 | 10.08 | 8.60 | 1.59E−03 | 1.48 | 4.4 |
| hsa-miR-96 | 2.78E−04 | 3.38E−04 | 7.62E−03 | 3.69 | 2.37 | 8.44E−04 | 1.32 | 3.7 |
| hsa-let-7a | 2.58E−04 | 8.01E−03 | 2.63E−03 | 8.95 | 7.69 | 1.51E−04 | 1.26 | 3.5 |
| hsa-miR-302b | 2.03E−01 | 1.11E−01 | 7.70E−03 | 2.61 | 1.51 | 1.28E−03 | 1.10 | 3.0 |
| hsa-let-7i | 2.21E−04 | 5.46E−02 | 7.97E−03 | 6.48 | 5.40 | 6.98E−04 | 1.07 | 2.9 |
| hsa-miR-29a | 1.21E−04 | 3.47E−04 | 3.39E−02 | 8.49 | 7.98 | 1.57E−03 | 0.52 | 1.7 |
| hsa-miR-31 | 1.18E−02 | 1.69E−03 | 8.47E−02 | 9.27 | 8.81 | 1.64E−03 | 0.46 | 1.6 |
| hsa-miR-30d | 6.33E−05 | 2.84E−07 | 7.65E−03 | 6.78 | 6.42 | 5.34E−04 | 0.37 | 1.4 |
| hsa-miR-106b | 1.40E−04 | 7.43E−02 | 1.20E−02 | 5.73 | 6.22 | 1.20E−03 | −0.49 | 1.6 |
| ambi-miR- | 7.91E−02 | 1.18E−04 | 3.94E−03 | 1.16 | 2.48 | 5.07E−05 | −1.32 | 3.7 |
| 7097 | | | | | | | | |
| hsa-miR-330 | 3.88E−04 | 8.51E−05 | 7.38E−03 | 1.40 | 3.03 | 9.27E−05 | −1.64 | 5.1 |

CIN-612 9E cell line before differentiation (CIN-612-9EM) vs CIN-612 9E cell line 48 hr after differentiation (CIN-612-9E48). Two miRNAs were expressed with a significant differential expression in CIN-612 9E cell line after 48 hr of induction of differentiation by methylcellulose (p-value (CIN-612-9E48-CIN-612-9EM)<0.00025974). Only hsa-miR-203 was up-regulated in CIN-612-9E48 by more than 5-fold (Table 10).

TABLE 10
miRNAs differentially expressed between CIN-612 9Ecells before differentiation and
48 hr after differentiation. CIN-612-9EM, CIN-612 9E monolayer cells before differentiation;
CIN-612-9E48, CIN-612 9E cells 48 hr after differentiation.

| | | | | | | p-value | ΔH | |
|---|---|---|---|---|---|---|---|---|
| | | | | | | (CIN-612- | (CIN-612- | |
| | p-value | p-value | p-value | Mean | Mean | 9E48 − CIN- | 9E48 − CIN- | Fold |
| miRNA | (Type) | (Treatment) | (Type * Treatment) | (CIN-612-9EM) | (CIN-612-9E48) | 612-9EM) | 612-9EM) | Change |
| hsa-miR-230 | 5.34E−02 | 2.52E−07 | 1.15E−02 | 7.02 | 9.86 | 7.07E−05 | 2.84 | 17.2 |
| hsa-miR-30d | 6.33E−05 | 2.84E−07 | 7.65E−03 | 6.87 | 7.39 | 1.46E−04 | 0.52 | 1.7 |

Comparison of the miRNA expression changes induced by differentiation in HPV-associated NHK against those induced by differentiation of uninfected NHK revealed that, in general, fewer miRNAs had significantly different expression levels in HPV-associated NHK following 48 hr of methylcellulose-induced differentiation. One miRNA, hsa-miR-203, exhibited significantly different expression following methylcellulose-induced differentiation in NHK and in all HPV-associated NHK evaluated here, including CIN-612 9E. hsa-miR-203 was expressed at levels 4.3-fold to 27-fold higher following cellular differentiation. Up-regulation of hsa-miR-203 may represent an important feature of keratinocyte differentiation that must be maintained in cells infected with HPV. In addition to hsa-miR-203, seven miRNAs (hsa-miR-194, -491, -224, -96, -132, -30e-5p and ambi-miR-7027) were up-regulated in NHK48 compared to NHKM but not differentially expressed in any of the HPV-associated cell lines after induction of differentiation. Similarly, five miRNAs (hsa-miR-130a, -197, -181b, -130b, and -503) were found down-regulated in NHK after induction of differentiation but not in the cell lines associated with HPV18 and HPV31, including CIN-612 9E after induction of differentiation. These miRNAs are most likely involved in the normal differentiation program of keratinocytes but are not necessary for the differentiation of HPV-infected cells.

Eight miRNAs (hsa-miR-30b, -193b, -491, -132, -200b, -200a, -495, -and -30d) were found only up-regulated (2-7 fold) in HPV31-associated NHK after induction of differentiation. However, none of these miRNAs was up-regulated in CIN-612 9E after induction of differentiation. hsa-miR-203 and hsa-miR-192 were up-regulated in NHK and in HPV31-associated NHK after induction of differentiation and hsa-miR-203 and hsa-miR-210 were up-regulated in HPV31- and HPV18-associated NHK but not in the other HPV-associated cell lines.

### Example 5

### miRNA Expression Differences Among NHK, HPV-Associated NHK, and HPV31-Associated Cell Line (CIN-612 9E) After 48 Hr of Induced Cell Differentiation

HPV31-associated NHK (NHK3148) vs NHK (NHK48) at 48 hr after differentiation. Comparison between HPV31-associated NHK and NHK cells, both harvested 48 hr after induction of differentiation, revealed only five miRNAs expressed with a significant differential expression (p-value (NHK3148-NHK48)<0.000909091) and at least a 2-fold change between NHK3148 and NHK48 (Table 11). All were expressed at a higher level in NHK3148 by at least 100-fold (hsa-miR-199a-AS and -199a), 30-fold (hsa-miR-214 and -145) or 10-fold (hsa-miR-143). These miRNAs were also observed in Example 3 above (Example 3, Table 2) to be the most differentially expressed miRNAs between NHK31M (monolayer cells) and NHKM (monolayer cells). However, the expression differences between the two cell lines were greater after 48 hr of methylcellulose-induced cell differentiation for hsa-miR-199a and -199a_AS, but were reduced for hsa-miR-214, -145, and -143. No miRNA was significantly down-regulated in HPV3148 cells. In particular, the absence of down-regulation of hsa-miR-503 can be explained by the observation that this miRNA is down-regulated after induction of differentiation, as shown in NHK48 versus NHKM, indicating that it is not further down-regulated in HPV3148 (Table 6),

TABLE 11
MicroRNAs differentially expressed between HPV31-associated NHK 48 hr after
differentiation and NHK 48 hr after differentiation. NHK3148, HPV31-associated NHK 48 hr
after differentiation; NHK48, NHK 48 hr after differentiation.

| | | | p-value | | | p-value | ΔH | |
|---|---|---|---|---|---|---|---|---|
| | p-value | p-value | (Type * | Mean | Mean | (NHK3148H − | (NHK3148H − | Fold |
| miRNA | (Type) | (Treatment) | Treatment) | (NHK48H) | (NHK3148H) | NHK48H) | NHK48H) | Change |
| hsa-miR-199a_AS | 4.17E−07 | 7.72E−02 | 2.54E−02 | 1.37 | 6.98 | 4.34E−07 | 5.61 | 272.0 |
| hsa-miR-199a | 5.94E−06 | 6.57E−01 | 6.47E−01 | 1.40 | 6.08 | 1.34E−05 | 4.69 | 108.5 |
| hsa-miR-214 | 3.04E−05 | 2.22E−01 | 5.15E−01 | 2.65 | 6.22 | 1.38E−04 | 3.57 | 35.6 |
| hsa-miR-145 | 4.39E−05 | 5.01E−01 | 5.29E−01 | 1.51 | 5.04 | 6.84E−05 | 3.53 | 34.1 |
| hsa-miR-143 | 1.22E−04 | 2.36E−01 | 4.03E−01 | 1.91 | 4.53 | 1.86E−04 | 2.62 | 13.8 |
| hsa-miR-30a_5p | 6.75E−06 | 2.48E−06 | 2.42E−02 | 7.39 | 7.75 | 2.98E−04 | 0.36 | 1.4 |

HPV18-associated NHK (NHK1848) vs NHK (NHK48) at 48 hr after differentiation. Eleven miRNAs were expressed with a significant differential expression between HPV18-associated NHK and NHK, 48 hr after induction of differentiation (p-value (NHK1848-NHK48)<0.00168831) (Table 12), corresponding to five up-regulated and two down-regulated miRNAs in NHK1848 compared to NHK48 cells by at least 2-fold (|ΔH|(NHK1848 vs NHK48)>0.69). The five miRNAs expressed at a higher level in NHK1848 cells are the same as those miRNAs observed above in Example 3 (Example 3, Table 3) that are up-regulated in NHK18M (monolayer cells) compared to NHKM (monolayer cells). However, the expression differences observed between the two cell lines were greater after 48 hr of methylcellulose-induced cell differentiation for hsa-miR-199a, -199a AS (>100-fold), hsa-miR-145 (>30-fold), and hsa-miR-143 (>10-fold) but was lower for hsa-miR-214 (>30-fold). The two down-regulated miRNAs in NHK1848, hsa-miR-424 and hsa-miR-96, were down-regulated by 3- to 13-fold in NHK1848 compared to NHK48. hsa-miR-503, which is down-regulated in non-infected NHK after induction of differentiation, was not further down-regulated in HPV18-associated NHK after induction of differentiation.

TABLE 12
MicroRNAs differentially expressed between HPV18-associated NHK 48 hr after
differentiation and NHK 48 hr after differentiation. NHK1848, HPV18-associated NHK 48 hr
after differentiation; NHK48, NHK 48 hr after differentiation.

| | | | p-value | | | p-value | □H | |
|---|---|---|---|---|---|---|---|---|
| | p-value | p-value | (Type * | Mean | Mean | (NHK1848 − | (NHK1848 − | Fold |
| miRNA | (Type) | (Treatment) | Treatment) | (NHK48) | (NHK1848) | NHK48) | NHK48) | Change |
| hsa-miR-199a_AS | 4.17E−07 | 7.72E−02 | 2.54E−02 | 1.37 | 6.74 | 5.88E−07 | 5.36 | 213.3 |
| hsa-miR-199a | 5.94E−06 | 6.57E−01 | 6.47E−01 | 1.40 | 6.19 | 1.15E−05 | 4.79 | 120.7 |
| hsa-miR-145 | 4.39E−05 | 5.01E−01 | 5.29E−01 | 1.51 | 5.09 | 6.33E−05 | 3.57 | 35.6 |
| hsa-miR-214 | 3.04E−05 | 2.22E−01 | 5.15E−01 | 2.65 | 6.16 | 1.52E−04 | 3.52 | 33.7 |
| hsa-miR-143 | 1.22E−04 | 2.36E−01 | 4.03E−01 | 1.91 | 4.84 | 9.23E−05 | 2.93 | 18.7 |
| hsa-miR-30a-5p | 6.75E−06 | 2.48E−06 | 2.42E−02 | 7.39 | 7.89 | 3.94E−05 | 0.50 | 1.6 |
| hsa-miR-30c | 4.71E−05 | 8.63E−06 | 2.56E−02 | 7.16 | 7.51 | 1.05E−03 | 0.35 | 1.4 |
| hsa-miR-200a | 3.03E−05 | 3.75E−06 | 5.47E−05 | 6.87 | 6.55 | 9.81E−04 | −0.32 | 1.4 |
| hsa-miR-200b | 1.94E−04 | 1.18E−04 | 1.30E−02 | 8.22 | 7.63 | 1.46E−03 | −0.59 | 1.8 |
| hsa-miR-96 | 2.78E−04 | 3.38E−04 | 7.62E−03 | 5.21 | 4.17 | 1.07E−03 | −1.04 | 2.8 |
| hsa-miR-424 | 3.21E−03 | 5.75E−01 | 6.05E−01 | 5.31 | 2.77 | 1.37E−03 | −2.54 | 12.7 |

HPV11-associated NHK (NHK1148) vs NHK (NHK48) at 48 hr after differentiation. Twenty-eight human miRNAs were expressed with a significant differential expression between HPV11-associated NHK and NHK, 48 hr after induction of differentiation (p-value (NHK1148-NHK48)<0.00415584), corresponding to five miRNAs expressed at a higher level and eighteen miRNAs expressed at a lower level in HPV11-associated NHK cells after differentiation, by at least 2-fold (|ΔH|(NHK1148 vs NHK48)>0.69) (Table 13). The miRNAs expressed at a higher level in NHK1148 compared to NHK48 cells were the same miRNAs that were observed in Example 3 above (Example 3, Table 4) to be expressed at a higher level in NHK11M (monolayer cells) compared to NHKM (monolayer cells). However, the expression differences observed between the two cell lines were greater after 48 hr of methylcellulose-induced cell differentiation for hsa-miR-199a-AS, -199a, -145, and -143 and ranged from more than a 100 for both hsa-miR-199a-AS and -199a, to between 10- and 35-fold for hsa-miR-145 and -143. It was reduced for hsa-miR-214 (>40-fold). Among the miRNAs expressed at a lower level in NHK1148 compared to NHK48, hsa-miR-19a was down-regulated more than 10-fold, and hsa-miR-196b was expressed between 5- and 10-fold lower in NHK1148 versus NHK48. In addition, seven miRNAs were down-regulated in HPV11-associated NHK before and after induction of differentiation by at least a 2-fold change, corresponding to hsa-let7d and hsa-miR-200b, -96, -25, -422, -19a, and -196b (Table 13).

TABLE 13
MicroRNAs differentially expressed between HPV11-associated NHK 48 hr after
differentiation and NHK 48 hr after differentiation. NHK1148, HPV11-associated NHK 48 hr
after differentiation; NHK48, NHK 48 hr after differentiation.

| | | | p-value | | | p-value | ΔH | |
|---|---|---|---|---|---|---|---|---|
| | p-value | p-value | (Type * | Mean | Mean | (NHK1148 − | (NHK1148 − | Fold |
| miRNA | (Type) | (Treatment) | Treatment) | (NHK48) | (NHK1148) | NHK48) | NHK48H) | Change |
| hsa-miR- | 4.17E−07 | 7.72E−02 | 2.54E−02 | 1.37 | 6.60 | 6.99E−07 | 5.23 | 186.6 |
| 199a_AS | | | | | | | | |
| hsa-miR-199a | 5.94E−06 | 6.57E−01 | 6.47E−01 | 1.40 | 6.09 | 1.32E−05 | 4.70 | 109.4 |
| hsa-miR-214 | 3.04E−05 | 2.22E−01 | 5.15E−01 | 2.65 | 6.44 | 9.49E−05 | 3.79 | 44.2 |
| hsa-miR-145 | 4.39E−05 | 5.01E−01 | 5.29E−01 | 1.51 | 4.99 | 7.57E−05 | 3.48 | 32.3 |
| hsa-miR-143 | 1.22E−04 | 2.36E−01 | 4.03E−01 | 1.91 | 4.49 | 2.07E−04 | 2.58 | 13.1 |
| hsa-miR-30d | 6.33E−05 | 2.84E−07 | 7.65E−03 | 7.02 | 6.78 | 2.13E−03 | −0.24 | 1.3 |
| hsa-miR-30c | 4.71E−05 | 8.63E−06 | 2.56E−02 | 7.16 | 6.74 | 3.60E−04 | −0.42 | 1.5 |
| hsa-miR-106b | 1.40E−04 | 7.43E−02 | 1.20E−02 | 6.27 | 5.73 | 2.11E−04 | −0.54 | 1.7 |
| hsa-miR-26a | 8.29E−04 | 5.78E−04 | 5.16E−01 | 8.24 | 7.62 | 5.98E−04 | −0.62 | 1.9 |
| hsa-miR-29a | 1.21E−04 | 3.47E−04 | 3.39E−02 | 9.18 | 8.49 | 8.33E−05 | −0.69 | 2.0 |
| hsa-miR-200a | 3.03E−05 | 3.75E−06 | 5.47E−05 | 6.87 | 6.10 | 3.56E−06 | −0.77 | 2.2 |
| hsa-let-7d | 2.04E−03 | 3.84E−02 | 7.50E−02 | 8.50 | 7.68 | 4.14E−03 | −0.82 | 2.3 |
| hsa-miR-375 | 1.21E−03 | 9.30E−01 | 1.25E−03 | 2.33 | 1.45 | 5.98E−04 | −0.88 | 2.4 |
| ambi-miR-7097 | 7.91E−02 | 1.18E−04 | 3.94E−03 | 2.06 | 1.16 | 1.53E−04 | −0.91 | 2.5 |
| hsa-miR-15b | 3.57E−04 | 1.29E−02 | 1.91E−01 | 6.93 | 5.97 | 3.06E−03 | −0.96 | 2.6 |
| hsa-miR-200b | 1.94E−04 | 1.18E−04 | 1.30E−02 | 8.22 | 7.22 | 6.07E−05 | −0.99 | 2.7 |
| hsa-miR-25 | 6.37E−04 | 3.55E−03 | 2.13E−01 | 6.45 | 5.36 | 4.00E−03 | −1.09 | 3.0 |
| hsa-miR-148b | 3.15E−03 | 8.61E−01 | 2.71E−01 | 3.87 | 2.75 | 2.20E−03 | −1.12 | 3.1 |
| hsa-miR-422a | 2.45E−04 | 7.87E−03 | 2.76E−01 | 5.15 | 3.96 | 3.18E−03 | −1.18 | 3.3 |
| hsa-miR-330 | 3.88E−04 | 8.51E−05 | 7.38E−03 | 2.64 | 1.40 | 1.44E−04 | −1.25 | 3.5 |
| hsa-miR-148a | 4.66E−03 | 4.68E−03 | 3.66E−01 | 6.03 | 4.68 | 4.02E−03 | −1.34 | 3.8 |
| hsa-miR-517_AS | 7.78E−02 | 8.88E−03 | 1.24E−01 | 2.19 | 0.80 | 2.26E−03 | −1.39 | 4.0 |
| hsa-miR-516-3p | 6.76E−01 | 1.61E−02 | 3.45E−02 | 2.17 | 0.76 | 3.50E−03 | −1.41 | 4.1 |
| hsa-miR-96 | 2.78E−04 | 3.38E−04 | 7.62E−03 | 5.21 | 3.69 | 1.06E−04 | −1.52 | 4.6 |
| hsa-miR-365 | 1.10E−02 | 9.68E−02 | 1.19E−01 | 4.82 | 3.20 | 1.47E−03 | −1.62 | 5.0 |
| hsa-miR-32 | 4.82E−04 | 7.91E−01 | 1.04E−01 | 2.19 | 0.43 | 7.48E−05 | −1.76 | 5.8 |
| hsa-miR-196b | 5.90E−05 | 9.15E−02 | 8.27E−02 | 4.26 | 2.31 | 9.35E−05 | −1.96 | 7.1 |
| hsa-miR-19a | 1.40E−03 | 7.37E−01 | 6.79E−01 | 5.48 | 3.08 | 4.84E−04 | −2.41 | 11.1 |

HPV31-associated CIN-612 9E cell line (CIN-612-9E48) vs NHK (NHK48) at 48 hr after differentiation. Eleven human miRNAs were expressed with a significant differential expression between CIN-612-9E48 and NHK48 (p-value (CIN-612-9E48-NHK48)<0.00155844), corresponding to six miRNAs expressed at higher levels and one miRNA expressed at a lower level in CIN-612-9E48 versus NHK48 by at least 2-fold (|□H|(CIN-612-9E48 vs NHK48)>0.69) (Table 14). Among these, hsa-miR-199a_AS was over-expressed by at least 100-fold in CIN-612-9E48 compared to NHK48; hsa-miR-199a was over-expressed by at least 50-fold, and hsa-miR-214, -145, and -143 were over-expressed between 10- and 30-fold in CIN-612-9E48 compared to NHK48 cells. Among these miRNAs, hsa-miR-199a differential expression was greater between CIN-612-9E48 versus NHK48 than between CIN-612-9EM compared with NHKM, whereas hsa-miR-199aAS, -214, -145, and -143 differential expressions were lower. In addition, hsa-miR-196a was the only miRNA down-regulated in CIN-612-9E48 cells compared to NHK48, with a lower expression level of 10-fold (Table 14).

TABLE 14
MicroRNAs differentially expressed between HPV31-associated CIN-612 9E cell line
48 hr after differentiation and NHK 48 hr after differentiation. CIN-612-9E48, HPV31-
associated CTN-612 9E cell line 48 hr after differentiation; NHK48, NHK 48 hr after
differentiation.

| | | | | | | p-value | ΔH | |
|---|---|---|---|---|---|---|---|---|
| | p-value | p-value | p-value | Mean | Mean | (CIN-612-9E48 vs | (CIN-612-9E48 − | Fold |
| miRNA | (Type) | (Treatment) | (Type * Treatment) | (NHK48) | (CIN-612-9E48) | NHK48) | NHK48) | Change |
| hsa-miR-199a_AS | 4.17E−07 | 7.72E−02 | 2.54E−02 | 1.37 | 6.55 | 2.99E−06 | 5.17 | 176.5 |
| hsa-miR-199a | 5.94E−06 | 6.57E−01 | 6.47E−01 | 1.40 | 5.78 | 7.85E−05 | 4.38 | 79.7 |
| hsa-miR-214 | 3.04E−05 | 2.22E−01 | 5.15E−01 | 2.65 | 5.98 | 7.24E−04 | 3.34 | 28.1 |
| hsa-miR-145 | 4.39E−05 | 5.01E−01 | 5.29E−01 | 1.51 | 4.70 | 4.65E−04 | 3.18 | 24.1 |
| hsa-miR-143 | 1.22E−04 | 2.36E−01 | 4.03E−01 | 1.91 | 4.37 | 9.22E−04 | 2.46 | 11.7 |
| hsa-miR-422b | 3.87E−03 | 9.42E−01 | 2.57E−02 | 5.71 | 6.88 | 7.96E−04 | 1.17 | 3.2 |
| hsa-miR-151 | 1.47E−04 | 5.04E−02 | 4.54E−02 | 4.74 | 5.43 | 1.28E−03 | 0.69 | 2.0 |
| hsa-miR-30d | 6.33E−05 | 2.84E−07 | 7.65E−03 | 7.02 | 7.39 | 4.72E−04 | 0.37 | 1.5 |
| hsa-miR-200a | 3.03E−05 | 3.75E−06 | 5.47E−05 | 6.87 | 6.41 | 3.71E−04 | −0.46 | 1.6 |
| hsa-miR-29a | 1.21E−04 | 3.47E−04 | 3.39E−02 | 9.18 | 8.58 | 6.93E−04 | −0.60 | 1.8 |
| hsa-miR-196a | 3.36E−04 | 3.02E−01 | 4.45E−01 | 5.91 | 3.61 | 2.47E−04 | −2.30 | 10.0 |

Common and different miRNAs differentially expressed between HPV-associated cell lines and HPV-negative NHK cells, after induction of differentiation for 48 h in methylcellulose. The data in Tables 11, 12, 13, and 14 demonstrate that five miRNAs (hsa-miR-199a, -199a-AS, -214, -145, and -143) are expressed at significantly higher levels in all HPV-associated NHK and in CIN-612 9E than in NHK, following 48 hr of methylcellulose-induced differentiation. These five miRNAs are also expressed at significantly higher levels in HPV-associated NHK than in NHK, in undifferentiated monolayer cells (Example 3, Tables 2, 3, 4, and 5). These miRNAs are also up-regulated in the HPV31-associated, low-grade, CIN-derived cell line (CIN-612 9E) compared to NHK, indicating that the effect is not an artifact due to the transfection of HPV genomes into NHK and selection of the transfected cells. Furthermore, the up-regulation of the miRNAs is independent of the oncogenic potential of the HPV type, as HPV31 and HPV18 are types associated with cervical cancer but HPV11 is associated with benign mucosal lesions that do not usually transform into cancer. Up-regulation of these miRNAs may represent a cellular host-response to the presence of HPV. However, this cellular host response is not the anti-viral response induced by interferon, as none of these miRNAs is up-regulated after treatment of the cells with interferon-beta (data not shown).

hsa-miR-503 is down-regulated in undifferentiated HPV18- and HPV31-associated cell lines, including CIN-612 9E, when compared to undifferentiated NHK. Down-regulation of hsa-miR-503 is no longer observed when HPV-18- and HPV-31 cell lines are compared with NHK after induction of differentiation. As hsa-miR-503 is down-regulated in non-infected NHK after differentiation, this observation indicates that the level of expression of hsa-miR-503 in HPV18- and HPV31-associated cell lines is not affected by differentiation. hsa-miR-503 is not differentially expressed between undifferentiated HPV11-associated NHK and NHK and is still not differentially expressed after induction of differentiation of these cells. This indicates that hsa-miR-503 is down-regulated in HPV11-associated NHK after induction of differentiation, as observed in NHK. Therefore, HPV11-associated NHK behaves as NHK regarding the expression level of hsa-miR-503.

Other differences were noticed between HPV11- and HPV18- or HPV31-associated cell lines after induction of differentiation. Seven miRNAs were down-regulated in HPV11-associated NHK compared to HPV18- and HPV31-associated cell lines, including CIN-612 9E, after induction of differentiation by at least 2-fold. These miRNAs correspond to ambi-miR-7097 and hsa-miR-106b, -16, -330, -196b, -422a, and -32. Among these, only hsa-miR-422a was also down-regulated in undifferentiated HPV11-associated NHK compared to undifferentiated HPV18- and HPV31-associated cell lines. In addition to these miRNAs, hsa-miR-30c, hsa-miR-25, and ambi-miR-7026 were down-regulated in HPV11-associated NHK compared to HPV18- and HPV31-associated NHK after induction of differentiation. These observations indicate that oncogenic and non oncogenic HPV types affect differently the normal differentiation program of keratinocytes. Importantly, no difference was observed between HPV18- and HPV31-associated cell lines, including CIN-612 9E, after induction of differentiation, further indicating that oncogenic HPV types share similar mechanisms, despite their diversity.

### Example 7

### Microarray Data Validation by QRT-PCR

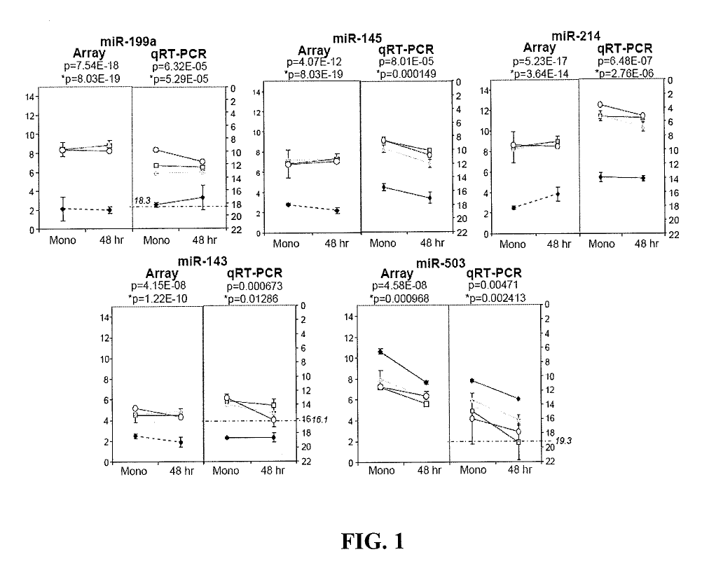

To verify array data, the inventors performed real-time qRT-PCR reactions to quantify four (hsa-miR-199a, -214, -145, and -143) of the five miRNAs most up-regulated between HPV-associated NHK or HPV31-associated CIN-612 9E cell line and NHK and to quantify hsa-miR-503, the most down-regulated miRNA in HPV-associated NHK compared to NHK. qRT-PCR reactions utilized 15 ng of input total RNA from HPV31-associated NHK, HPV18-associated NHK, HPV31-associated CIN-612 9E cell line, and NHK and were performed using TaqMan® MicroRNA Assays (Applied Biosystems; Foster City, Calif., USA) according to the manufacturer's instructions. Reaction mixtures were incubated in a 7900HT Fast Real-Time PCR System (Applied Biosystems). Initial data analysis was done using the 7500 Fast System SDS 2.3 software. miRNA expression data obtained with primer sets specific for the indicated miRNAs were normalized to miR-16 expression level for each sample (ΔCT=miRNA Ct-miR-16 Ct). Comparisons of array and qRT-PCR data are illustrated in FIG. 1. qRT-PCR results confirmed the results of the array analyses and demonstrated that qRT-PCR is a useful method for identifying miRNA expression changes in HPV-infected human keratinocytes.

TABLE 15
miRNAs Sequences

| | | |
|---|---|---|
| miRNA | Mature sequence | Precursor |

| | | |
|---|---|---|
| ambi_miR_7027 | AAAUGGUGCCCUAGU | |
| | GACUAC | |
| | (SEQ ID NO: 1) | |
| ambi_miR_7076 | AAUCCUUGGAACCUA | CUUGAAUCCUUGGAACCUA |
| | GGUGUGAGU | GGUGUGAGUGCUAUUUCAG |
| | (SEQ ID NO: 2) | UGCAACACACCUAUUCAAG |
| | | GAUUCAAA |
| | | (SEQ ID NO: 3) |
| ambi_miR_7086 | ACCACUGACCGUUGA | |
| | CUGUACC | |
| | (SEQ ID NO: 4) | |
| hsa_let_7a | UGAGGUAGUAGGUUG | UGGGAUGAGGUAGUAGGUU |
| | UAUAGUU | GUAUAGUUUUAGGGUCACA |
| | (SEQ ID NO: 5) | CCCACCACUGGGAGAUAAC |
| | | UAUACAAUCUACUGUCUUU |
| | | CCUA |
| | | (SEQ ID NO: 6) |
| hsa_let_7c | UGAGGUAGUAGGUUG | GCAUCCGGGUUGAGGUAGU |
| | UAUGGUU | AGGUUGUAUGGUUUAGAGU |
| | (SEQ ID NO: 7) | UACACCCUGGGAGUUAACU |
| | | GUACAACCUUCUAGCUUUC |
| | | CUUGGAGC |
| | | (SEQ ID NO: 8) |
| hsa_let 7d | AGAGGUAGUAGGUUG | CCUAGGAAGAGGUAGUAGG |
| | CAUAGUU | UUGCAUAGUUUUAGGGCAG |
| | (SEQ ID NO: 9) | GGAUUUUGCCCACAAGGAG |
| | | GUAACUAUACGACCUGCUG |
| | | CCUUUCUUAGG |
| | | (SEQ ID NO: 10) |
| hsa_let_7e | UGAGGUAGGAGGUUG | CCCGGGCUGAGGUAGGAGG |
| | UAUAGUU | UUGUAUAGUUGAGGAGGAC |
| | (SEQ ID NO: 11) | ACCCAAGGAGAUCACUAUA |
| | | CGGCCUCCUAGGUUUCGCC |
| | | AGG |
| | | (SEQ ID NO: 12) |
| hsa_let_7g | UGAGGUAGUAGUUUG | AGGCUGAGGUAGUAGUUUG |
| | UACAGUU | UACAGUUUGAGGGUCUAUG |
| | (SEQ ID NO: 13) | AUACCACCCGGUACAGGAG |
| | | AUAACUGUACAGGCCACUG |
| | | CCUUGCCA |
| | | (SEQ ID NO: 14) |
| hsa_let_7i | UGAGGUAGUAGUUUG | CUGGCUGAGGUAGUAGUUU |
| | UGCUGUU | GUGCUGUUGGUCGGGUUGU |
| | (SEQ ID NO: 15) | GACAUUGCCCGCUGUGGAG |
| | | AUAACUGCGCAAGCUACUG |
| | | CCUUGCUA |
| | | (SEQ ID NO: 16) |
| hsa_miR_106b | UAAAGUGCUGACAGU | CCUGCCGGGGCUAAAGUGC |
| | GCAGAU | UGACAGUGCAGAUAGUGGU |
| | (SEQ ID NO: 17) | CCUCUCCGUGCUACCGCAC |
| | | UGUGGGUACUUGCUGCUCG |
| | | AGCAGG |
| | | (SEQ ID NO: 18) |
| hsa_miR_130a | UUCACAUUGUGCUAC | UGCUGCUGGCCAGAGCUCU |
| | UGUCUGC | UUUCACAUUGUGCUACUGU |
| | (SEQ ID NO: 19) | CUGCACCUGUCACUAGCAG |
| | | UGCAAUGUUAAAAGGGCAU |
| | | UGGCCGUGUAGUG |
| | | (SEQ ID NO: 20) |
| hsa_miR_130b | CAGUGCAAUGAUGAA | GGCCUGCCCGACACUCUUU |
| | AGGGCAU | CCCUGUUGCACUACUAUAG |
| | (SEQ ID NO: 21) | GCCGCUGGGAAGCAGUGCA |
| | | AUGAUGAAAGGGCAUCGGU |
| | | CAGGUC |
| | | (SEQ ID NO: 22) |
| hsa_miR_132 | UAACAGUCUACAGCC | CCGCCCCCGCGUCUCCAGG |
| | AUGGUCG | GCAACCGUGGCUUUCGAUU |
| | (SEQ ID NO: 23) | GUUACUGUGGGAACUGGAG |
| | | GUAACAGUCUACAGCCAUG |
| | | GUCGCCCCGCAGCACGCCC |
| | | ACGCGC |
| | | (SEQ ID NO: 24) |
| hsa_miR_142_3p | UGUAGUGUUUCCUAC | GACAGUGCAGUCACCCAUA |
| | UUUAUGGA | AAGUAGAAAGCACUACUAA |
| | (SEQ ID NO: 25) | CAGCACUGGAGGGUGUAGU |
| | | GUUUCCUACUUUAUGGAUG |
| | | AGUGUACUGUG |
| | | (SEQ ID NO: 26) |
| hsa_miR_143 | UGAGAUGAAGCACUG | GCGCAGCGCCCUGUCUCCC |
| | UAGCUC | AGCCUGAGGUGCAGUGCUG |
| | (SEQ ID NO: 27) | CAUCUCUGGUCAGUUGGGA |
| | | GUCUGAGAUGAAGCACUGU |
| | | AGCUCAGGAAGAGAGAAGU |
| | | UGUUCUGCAGC |
| | | (SEQ ID NO: 28) |
| hsa_miR_145 | GUCCAGUUUUCCCAG | CACCUUGUCCUCACGGUCC |
| | GAAUCCCU | AGUUUUCCCAGGAAUCCCU |
| | (SEQ ID NO: 29) | UAGAUGCUAAGAUGGGGAU |
| | | UCCUGGAAAUACUGUUCUU |
| | | GAGGUCAUGGUU |
| | | (SEQ ID NO: 30) |
| hsa_miR_148a | UCAGUGCACUACAGA | GAGGCAAAGUUCUGAGACA |
| | ACUUUGU | CUCCGACUCUGAGUAUGAU |
| | (SEQ ID NO: 31) | AGAAGUCAGUGCACUACAG |
| | | AACUUUGUCUC |
| | | (SEQ ID NO: 32) |
| hsa_miR_148b | UCAGUGCAUCACAGA | CAAGCACGAUUAGCAUUUG |
| | ACUUUGU | AGGUGAAGUUCUGUUAUAC |
| | (SEQ ID NO: 33) | ACUCAGGCUGUGGCUCUCU |
| | | GAAAGUCAGUGCAUCACAG |
| | | AACUUUGUCUCGAAAGCUU |
| | | UCUA |
| | | (SEQ ID NO: 34) |
| hsa_miR_151 | UCGAGGAGCUCACAG | UUUCCUGCCCUCGAGGAGC |
| | UCUAGU | UCACAGUCUAGUAUGUCUC |
| | (SEQ ID NO: 35) | AUCCGCUACUAGACUGAAG |
| | | CUGCUUGAGGACAGGGAUG |
| | | GUCAUACUCACCUC |
| | | (SEQ ID NO: 36) |
| hsa_miR_15b | UAGCAGCACAUCAUG | UUGAGGCCUUAAAGUACUG |
| | GUUUACA | UAGCAGCACAUCAUGGUUU |
| | (SEQ ID NO: 37) | ACAUGCUACAGUCAAGAUG |
| | | CGAAUCAUUAUUUGCUGCU |
| | | CUAGAAAUUUAAGGAAAUU |
| | | CAU |
| | | (SEQ ID NO: 38) |
| hsa_miR_16 | UAGCAGCACGUAAAU | GUCAGCAGUGCCUUAGCAG |
| | AUUGGCG | CACGUAAAUAUUGGCGUUA |
| | (SEQ ID NO: 39) | AGAUUCUAAAAUUAUCUCC |
| | | AGUAUUAACUGUGCUGCUG |
| | | AAGUAAGGUUGA |
| | | (SEQ ID NO: 40) |
| hsa_miR_181b | AACAUUCAUUGCUGU | CCUGUGCAGAGAUUAUUUU |
| | CGGUGGG | UUAAAAGGUCACAAUCAAC |
| | (SEQ ID NO: 41) | AUUCAUUGCUGUCGGUGGG |
| | | UUGAACUGUGUGGACAAGC |
| | | UCACUGAACAAUGAAUGCA |
| | | ACUGUGGCCCCGCUU |
| | | (SEQ ID NO: 42) |
| hsa_miR_192 | CUGACCUAUGAAUUG | GCCGAGACCGAGUGCACAG |
| | ACAGCC | GGCUCUGACCUAUGAAUUG |
| | (SEQ ID NO: 43) | ACAGCCAGUGCUCUCGUCU |
| | | CCCCUCUGGCUGCCAAUUC |
| | | CAUAGGUCACAGGUAUGUU |
| | | CGCCUCAAUGCCAGC |
| | | (SEQ ID NO: 44) |
| hsa_miR_193b | AACUGGCCCUCAAAG | GUGGUCUCAGAAUCGGGGU |
| | UCCCGCU | UUUGAGGGCGAGAUGAGUU |
| | (SEQ ID NO: 45) | UAUGUUUUAUCCAACUGGC |
| | | CCUCAAAGUCCGGCUUUUG |
| | | GGGUCAU |
| | | (SEQ ID NO: 46) |
| hsa_miR_194 | UGUAACAGCAACUCC | AUGGUGUUAUCAAGUGUAA |
| | AUGUGGA | CAGCAACUCCAUGUGGACU |
| | (SEQ ID NO: 47) | GUGUACCAAUUUCCAGUGG |
| | | AGAUGCUGUUACUUUUGAU |
| | | GGUUACCAA |
| | | (SEQ ID NO: 48) |
| hsa_miR_196a | UAGGUAGUUUCAUGU | GUGAAUUAGGUAGUUUCAU |
| | UGUUGGG | GUUGUUGGGCCUGGGUUUC |
| | (SEQ ID NO: 49) | UGAACACAACAACAUUAAA |
| | | CCACCCGAUUCAC |
| | | (SEQ ID NO: 50) |
| hsa_miR_196b | UAGGUAGUUUCCUGU | ACUGGUCGGUGAUUUAGGU |
| | UGUUGGG | AGUUUCCUGUUGUUGGGAU |
| | (SEQ ID NO: 51) | CCACCUUUCUCUCGACAGC |
| | | ACGACACUGCCUUCAUUAC |
| | | UUCAGUUG |
| | | (SEQ ID NO: 52) |
| hsa_miR_197 | UUCACCACCUUCUCC | GGCUGUGCCGGGUAGAGAG |
| | ACCCAGC | GGCAGUGGGAGGUAAGAGC |
| | (SEQ ID NO: 53) | UCUUCACCCUUCACCACCU |
| | | UCUCCACCCAGCAUGGCC |
| | | (SEQ ID NO: 54) |
| hsa_miR_199a | CCCAGUGUUCAGACU | GCCAACCCAGUGUUCAGAC |
| | ACCUGUUC | UACCUGUUCAGGAGGCUCU |
| | (SEQ ID NO: 55) | CAAUGUGUACAGUAGUCUG |
| | | CACAUUGGUUAGGC |
| | | (SEQ ID NO: 56) |
| hsa_miR_19a | UGUGCAAAUCUAUGC | GCAGUCCUCUGUUAGUUUU |
| | AAAACUGA | GCAUAGUUGCACUACAAGA |
| | (SEQ ID NO: 57) | AGAAUGUAGUUGUGCAAAU |
| | | CUAUGCAAAACUGAUGGUG |
| | | GCCUGC |
| | | (SEQ ID NO: 58) |
| hsa_miR_19b | UGUGCAAAUCCAUGC | CACUGUUCUAUGGUUAGUU |
| | AAAACUGA | UUGCAGGUUUGCAUCCAGC |
| | (SEQ ID NO: 59) | UGUGUGAUAUUCUGCUGUG |
| | | CAAAUCCAUGCAAAACUGA |
| | | CUGUGGUAGUG |
| | | (SEQ ID NO: 60) |
| hsa_miR_200a | UAACACUGUCUGGUA | CCGGGCCCCUGUGAGCAUC |
| | ACGAUGU | UUACCGGACAGUGCUGGAU |
| | (SEQ ID NO: 61) | UUCCCAGCUUGACUCUAAC |
| | | ACUGUCUGGUAACGAUGUU |
| | | CAAAGGUGACCCGC |
| | | (SEQ ID NO: 62) |
| hsa_miR_200b | UAAUACUGCCUGGUA | CCAGCUCGGGCAGCCGUGG |
| | AUGAUGA | CCAUCUUACUGGGCAGCAU |
| | (SEQ ID NO: 63) | UGGAUGGAGUCAGGUCUCU |
| | | AAUACUGCCUGGUAAUGAU |
| | | GACGGCGGAGCCCUGCACG |
| | | (SEQ ID NO: 64) |
| hsa_miR_203 | GUGAAAUGUUUAGGA | GUGUUGGGGACUCGCGCGC |
| | CCACUAG | UGGGUCCAGUGGUUCUUAA |
| | (SEQ ID NO: 65) | CAGUUCAACAGUUCUGUAG |
| | | CGCAAUUGUGAAAUGUUUA |
| | | GGACCACUAGACCCGGCGG |
| | | GCGCGGCGACAGCGA |
| | | (SEQ ID NO: 66) |
| hsa_miR_20a | UAAAGUGCUUAUAGU | GUAGCACUAAAGUGCUUAU |
| | GCAGGUAG | AGUGCAGGUAGUGUUUAGU |
| | (SEQ ID NO: 67) | UAUCUACUGCAUUAUGAGC |
| | | ACUUAAAGUACUGC |
| | | (SEQ ID NO: 68) |
| hsa_miR_210 | CUGUGCGUGUGACAG | ACCCGGCAGUGCCUCCAGG |
| | CGGCUGA | CGCAGGGCAGCCCCUGCCC |
| | (SEQ ID NO: 69) | ACCGCACACUGCGCUGCCC |
| | | CAGACCCACUGUGCGUGUG |
| | | ACAGCGGCUGAUCUGUGCC |
| | | UGGGCAGCGCGACCG |
| | | (SEQ ID NO: 70) |
| hsa_miR_214 | ACAGCAGGCACAGAC | GGCCUGGCUGGACAGAGUU |
| | AGGCAGU | GUCAUGUGUCUGCCUGUCU |
| | (SEQ ID NO: 71) | ACACUUGCUGUGCAGAACA |
| | | UCCGCUCACCUGUACAGCA |
| | | GGCACAGACAGGCAGUCAC |
| | | AUGACAACCCAGCGU |
| | | (SEQ ID NO: 72) |
| hsa_miR_22 | AAGCUGCCAGUUGAA | GGCUGAGCCGCAGUAGUUC |
| | GAACUGU | UUCAGUGGCAAGCUUUAUG |
| | (SEQ ID NO: 73) | UCCUGACCCAGCUAAAGCU |
| | | GCCAGUUGAAGAACUGUUG |
| | | CCCUCUGCC |
| | | (SEQ ID NO: 74) |
| hsa_miR_224 | CAAGUCACUAGUGGU | GGGCUUUCAAGUCACUAGU |
| | UCCGUU | GGUUCCGUUUAGUAGAUGA |
| | (SEQ ID NO: 75) | UUGUGCAUUGUUUCAAAAU |
| | | GGUGCCCUAGUGACUACAA |
| | | AGCCC |
| | | (SEQ ID NO: 76) |
| hsa_miR_25 | CAUUGCACUUGUCUC | GGCCAGUGUUGAGAGGCGG |
| | GGUCUGA | AGACUUGGGCAAUUGCUGG |
| | (SEQ ID NO: 77) | ACGCUGCCCUGGGCAUUGC |
| | | ACUUGUCUCGGUCUGACAG |
| | | UGCCGGCC |
| | | (SEQ ID NO: 78) |
| hsa_miR_26a | UUCAAGUAAUCCAGG | GUGGCCUCGUUCAAGUAAU |
| | AUAGGCU | CCAGGAUAGGCUGUGCAGG |
| | (SEQ ID NO: 79) | UCCCAAUGGGCCUAUUCUU |
| | | GGUUACUUGCACGGGGACG |
| | | C |
| | | (SEQ ID NO: 80) |
| hsa_miR_27a | UUCACAGUGGCUAAG | CUGAGGAGCAGGGCUUAGC |
| | UUCCGC | UGCUUGUGAGCAGGGUCCA |
| | (SEQ ID NO: 81) | CACCAAGUCGUGUUCACAG |
| | | UGGCUAAGUUCCGCCCCCC |
| | | AG |
| | | (SEQ ID NO: 82) |
| hsa_miR_27b | UUCACAGUGGCUAAG | ACCUCUCUAACAAGGUGCA |
| | UUCUGC | GAGCUUAGCUGAUUGGUGA |
| | (SEQ ID NO: 83) | ACAGUGAUUGGUUUCCGCU |
| | | UUGUUCACAGUGGCUAAGU |
| | | UCUGCACCUGAAGAGAAGG |
| | | UG |
| | | (SEQ ID NO: 84) |
| hsa_miR_29a | UAGCACCAUCUGAAA | AUGACUGAUUUCUUUUGGU |
| | UCGGUUA | GUUCAGAGUCAAUAUAAUU |
| | (SEQ ID NO: 85) | UUCUAGCACCAUCUGAAAU |
| | | CGGUUAU |
| | | (SEQ ID NO: 86) |
| hsa_miR_30a_5p | UGUAAACAUCCUCGA | GCGACUGUAAACAUCCUCG |
| | CUGGAAG | ACUGGAAGCUGUGAAGCCA |
| | (SEQ ID NO: 87) | CAGAUGGGCUUUCAGUCGG |
| | | AUGUUUGCAGCUGC |
| | | (SEQ ID NO: 88) |
| hsa_miR_30b | UGUAAACAUCCUACA | ACCAAGUUUCAGUUCAUGU |
| | CUCAGCU | AAACAUCCUACACUCAGCU |
| | (SEQ ID NO: 89) | GUAAUACAUGGAUUGGCUG |
| | | GGAGGUGGAUGUUUACUUC |
| | | AGCUGACUUGGA |
| | | (SEQ ID NO: 90) |
| hsa_miR_30c | UGUAAACAUCCUACA | ACCAUGCUGUAGUGUGUGU |
| | CUCUCAGC | AAACAUCCUACACUCUCAG |
| | (SEQ ID NO: 91) | CUGUGAGCUCAAGGUGGCU |
| | | GGGAGAGGGUUGUUUACUC |
| | | CUUCUGCCAUGGA |
| | | (SEQ ID NO: 92) |
| hsa_miR_30d | UGUAAACAUCCCCGA | GUUGUUGUAAACAUCCCCG |
| | CUGGAAG | ACUGGAAGCUGUAAGACAC |
| | (SEQ ID NO: 93) | AGCUAAGCUUUCAGUCAGA |
| | | UGUUUGCUGCUAC |
| | | (SEQ ID NO: 94) |
| hsa_miR_30e5p | UGUAAACAUCCUUGA | GGGCAGUCUUUGCUACUGU |
| | CUGGAAG | AAACAUCCUUGACUGGAAG |
| | (SEQ ID NO: 95) | CUGUAAGGUGUUCAGAGGA |
| | | GCUUUCAGUCGGAUGUUUA |
| | | CAGCGGCAGGCUGCCA |
| | | (SEQ ID NO: 96) |
| hsa_miR_31 | AGGCAAGAUGCUGGC | GGAGAGGAGGCAAGAUGCU |
| | AUAGCU | GGCAUAGCUGUUGAACUGG |
| | (SEQ ID NO: 97) | GAACCUGCUAUGCCAACAU |
| | | AUUGCCAUCUUUCC |
| | | (SEQ ID NO: 98) |
| hsa_miR_34a | UGGCAGUGUCUUAGC | GGCCAGCUGUGAGUGUUUC |
| | UGGUUGU | UUUGGCAGUGUCUUAGCUG |
| | (SEQ ID NO: 99) | GUUGUUGUGAGCAAUAGUA |
| | | AGGAAGCAAUCAGCAAGUA |
| | | UACUGCCCUAGAAGUGCUG |
| | | CACGUUGUGGGGCCC |
| | | (SEQ ID NO: 100) |
| hsa_miR_365 | UAAUGCCCCUAAAAA | ACCGCAGGGAAAAUGAGGG |
| | UCCUUAU | ACUUUUGGGGGCAGAUGUG |
| | (SEQ ID NO: 101) | UUUGCAUUCCACUAUCAUA |
| | | AUGCCCCUAAAAAUCCUUA |
| | | UUGCUCUUGCA |
| | | (SEQ ID NO: 102) |
| hsa_miR_379 | UGGUAGACUAUGGAA | AGAGAUGGUAGACUAUGGA |
| | CGUAGG | ACGUAGGCGUUAUGAUUUC |
| | (SEQ ID NO: 103) | UGACCUAUGUAACAUGGUC |
| | | CACUAACUCU |
| | | (SEQ ID NO: 104) |
| hsa_miR_422a | ACUGGACUUAGGGUC | GAGAGAAGCACUGGACUUA |
| | AGAAGGC | GGGUCAGAAGGCCUGAGUC |
| | (SEQ ID NO: 105) | CUCUCUGCUGCAGAUGGGC |
| | | UCUCUGUCCCUGAGCCAAG |
| | | CUUUGUCCUCCCUGG |
| | | (SEQ ID NO: 106) |
| hsa_miR_422b | ACUGGACUUGGAGUC | AGGGCUGCUGACUCCAGGU |
| | AGAAGG | CCUGUGUGUUACCUAGAAA |
| | (SEQ ID NO: 107) | UAGCACUGGACUUGGAGUC |
| | | AGAAGGCCU |
| | | (SEQ ID NO: 108) |
| hsa_miR_424 | CAGCAGCAAUUCAUG | CGAGGGGAUACAGCAGCAA |
| | UUUUGAA | UUCAUGUUUUGAAGUGUUC |
| | (SEQ ID NO: 109) | UAAAUGGUUCAAAACGUGA |
| | | GGCGCUGCUAUACCCCCUC |
| | | GUGGGGAAGGUAGAAGGUG |
| | | GGG |
| | | (SEQ ID NO: 110) |
| hsa_miR_491 | AGUGGGGAACCCUUC | UUGACUUAGCUGGGUAGUG |
| | CAUGAGG | GGGAACCCUUCCAUGAGGA |
| | (SEQ ID NO: 111) | GUAGAACACUCCUUAUGCA |
| | | AGAUUCCCUUCUACCUGGC |
| | | UGGGUUGG |
| | | (SEQ ID NO: 112) |
| hsa_miR_495 | AAACAAACAUGGUG | UGGUACCUGAAAAGAAGUU |
| | CACUUCUU | GCCCAUGUUAUUUUCGCUU |
| | (SEQ ID NO: 113) | UAUAUGUGACGAAACAAAG |
| | | AUGGUGCACUUCUUUUUCG |
| | | GUAUCA |
| | | (SEQ ID NO: 114) |
| hsa_miR_503 | UAGCAGCGGGAACAG | UGCCCUAGCAGCGGGAACA |
| | UUCUGCAG | GUUCUGCAGUGAGCGAUCG |
| | (SEQ ID NO: 115) | GUGCUCUGGGGUAUUGUUU |
| | | CCGCUGCCAGGGUA |
| | | (SEQ ID NO: 116) |
| hsa_miR_92 | UAUUGCACUUGUCCC | CUUUCUACACAGGUUGGGA |
| | GGCCUGU | UCGGUUGCAAUGCUGUGUU |
| | (SEQ ID NO: 117) | UCUGUAUGGUAUUGCACUU |
| | | GUCCCGGCCUGUUGAGUUU |
| | | GG |
| | | (SEQ ID NO: 118) |
| hsa_miR_96 | UUUGGCACUAGCACA | UGGCCGAUUUUGGCACUAG |
| | UUUUUGCU | CACAUUUUUGCUUGUGUCU |
| | (SEQ ID NO: 119) | CUCCGCUCUGAGCAAUCAU |
| | | GUGCAGUGCCAAUAUGGGA |
| | | AA |
| | | (SEQ ID NO: 120) |
| mmu_miR_155 | UUAAUGCUAAUUGUG | CUGUUAAUGCUAAUUGUGA |
| | AUAGGGGU | UAGGGGUUUUGGCCUCUGA |
| | (SEQ ID NO: 121) | CUGACUCCUACCUGUUAGC |
| | | AUUAACAG |
| | | (SEQ ID NO: 122) |
| mmu_miR_192 | CUGACCUAUGAAUUG | CGUGCACAGGGCUCUGACC |
| | ACAGCC | UAUGAAUUGACAGCCAGUA |
| | (SEQ ID NO: 123) | CUCUUUUCUCUCCUCUGGC |
| | | UGCCAAUUCCAUAGGUCAC |
| | | AGGUAUGUUCACC |
| | | (SEQ ID NO: 124) |
| mmu_miR_199b | ACAGUAGUCUGCACA | CCAGAGGAUACCUCCACUC |
| | UUGGUUA | CGUCUACCCAGUGUUUAGA |
| | (SEQ ID NO: 125) | CUACCUGUUCAGGACUCCC |
| | | AAAUUGUACAGUAGUCUGC |
| | | ACAUUGGUUAGGCUGGGCU |
| | | GGGUUAGACCCUCGG |
| | | (SEQ ID NO: 126) |
| mmu_miR_202 | UUCCUAUGCAUAUAC | GUUCCUUUUUCCUAUGCAU |
| | UUCUUU | AUACUUCUUUGUGGAUCUG |
| | (SEQ ID NO: 127) | GUCUAAAGAGGUAUAGCGC |
| | | AUGGGAAGAUGGAGC |
| | | (SEQ ID NO: 128) |
| mmu_miR_292_5p | ACUCAAACUGGGGGC | CAGCCUGUGAUACUCAAAC |
| | UCUUUUG | UGGGGGCUCUUUUGGAUUU |
| | (SEQ ID NO: 129) | UCAUCGGAAGAAAAGUGCC |
| | | GCCAGGUUUUGAGUGUCAC |
| | | CGGUUG |
| | | (SEQ ID NO: 130) |
| mmu_miR_337 | GAACGGCGUCAUGCA | CAGUGUAGUGAGAAGUUGG |
| | GGAGUU | GGGGUGGGAACGGCGUCAU |
| | (SEQ ID NO: 131) | GCAGGAGUUGAUUGCACAG |
| | | CCAUUCAGCUCCUAUAUGA |
| | | UGCCUUUCUUCACCCCCUU |
| | | CA |
| | | (SEQ ID NO: 132) |
| mmu_miR_409 | AGGUUACCCGAGCAA | UGGUACUCGGAGAGAGGUU |
| | CUUUGCAU | ACCCGAGCAACUUUGCAUC |
| | (SEQ ID NO: 133) | UGGAGGACGAAUGUUGCUC |
| | | GGUGAACCCCUUUUCGGUA |
| | | UCA |
| | | (SEQ ID NO: 134) |
| hsa_miR_199a_ | UACAGUAGUCUGCAC | |
| AS | AUUGGUU | |
| | (SEQ ID NO: 135) | |

### REFERENCES

The following references, to the extent that they provide exemplary procedural or other details supplementary to those set forth herein, are specifically incorporated herein by reference.
U.S. Pat. No. 4,337,063
U.S. Pat. No. 4,404,289
U.S. Pat. No. 4,405,711
U.S. Pat. No. 4,659,774
U.S. Pat. No. 4,682,195
U.S. Pat. No. 4,683,202
U.S. Pat. No. 4,704,362
U.S. Pat. No. 4,816,571
U.S. Pat. No. 4,959,463
U.S. Pat. No. 5,141,813
U.S. Pat. No. 5,143,854
U.S. Pat. No. 5,202,231
U.S. Pat. No. 5,214,136
U.S. Pat. No. 5,221,619
U.S. Pat. No. 5,223,618
U.S. Pat. No. 5,242,974
U.S. Pat. No. 5,264,566
U.S. Pat. No. 5,268,486
U.S. Pat. No. 5,288,644
U.S. Pat. No. 5,324,633
U.S. Pat. No. 5,378,825
U.S. Pat. No. 5,384,261
U.S. Pat. No. 5,405,783
U.S. Pat. No. 5,412,087
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,786
U.S. Pat. No. 5,468,613
U.S. Pat. No. 5,470,710
U.S. Pat. No. 5,470,967
U.S. Pat. No. 5,472,672
U.S. Pat. No. 5,480,980
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,580,726
U.S. Pat. No. 5,580,732
U.S. Pat. No. 5,583,013
U.S. Pat. No. 5,593,839
U.S. Pat. No. 5,599,672
U.S. Pat. No. 5,599,695
U.S. Pat. No. 5,602,240
U.S. Pat. No. 5,602,244
U.S. Pat. No. 5,610,287
U.S. Pat. No. 5,610,289
U.S. Pat. No. 5,614,617
U.S. Pat. No. 5,623,070
U.S. Pat. No. 5,624,711
U.S. Pat. No. 5,631,134
U.S. Pat. No. 5,637,683
U.S. Pat. No. 5,639,603
U.S. Pat. No. 5,645,897
U.S. Pat. No. 5,652,099
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,670,663
U.S. Pat. No. 5,672,697
U.S. Pat. No. 5,681,947
U.S. Pat. No. 5,695,940
U.S. Pat. No. 5,700,637
U.S. Pat. No. 5,700,922
U.S. Pat. No. 5,705,629
U.S. Pat. No. 5,708,154
U.S. Pat. No. 5,714,606
U.S. Pat. No. 5,728,525
U.S. Pat. No. 5,744,305
U.S. Pat. No. 5,763,167
U.S. Pat. No. 5,777,092
U.S. Pat. No. 5,792,847
U.S. Pat. No. 5,800,992
U.S. Pat. No. 5,807,522
U.S. Pat. No. 5,830,645
U.S. Pat. No. 5,837,196
U.S. Pat. No. 5,847,219
U.S. Pat. No. 5,858,988
U.S. Pat. No. 5,859,221
U.S. Pat. No. 5,871,928
U.S. Pat. No. 5,872,232
U.S. Pat. No. 5,876,932
U.S. Pat. No. 5,886,165
U.S. Pat. No. 5,919,626
U.S. Pat. No. 6,004,755
U.S. Pat. No. 6,087,102
U.S. Pat. No. 6,251,666
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Pat. No. 6,723,509
U.S. patent application Ser. No. 09/545,207
U.S. patent application Ser. No. 10/667,126
U.S. patent application Ser. No. 11/141,707
U.S. patent application Ser. No. 11/273,640
U.S. Patent Prov. Appn. Ser. No. 60/575,743
U.S. Patent Prov. Appn. Ser. No. 60/649,584
U.S. Patent Prov. Appn. Ser. No. 60/826,173
Andersen et al., Cancer Res., 64(15):5245-5250, 2004.
Arap et al., Cancer Res., 55(6): 1351-1354, 1995.
Austin-Ward and Villaseca, Revista Medica de Chile, 126(7):838-845, 1998.
Bedell et al., J Virol., 65(5):2254-60, 1991.
Benjamini, J. Royal Statistical Society, Series B., 57:289-300, 1995.
Bosch and de Sanjose, Dis Markers., 23(4):213-27, 2007.
Bosch et al., J. Natl. Cancer Inst., 87(11):796-802, 1995.
Bukowski et al., Clinical Cancer Res., 4(10):2337-2347, 1998.
Cai et al., J. Virol., 80(21):10890-3, 2006.
Caldas et al., Cancer Res., 54:3568-3573, 1994.
Calin and Croce, Nat. Rev. Cancer, 6(11):857-866, 2006.
Carrington and Ambros, Science, 301(5631):336-338, 2003.
Chen et al. Mol. Endocrinol., 19:441-458, 2005.
Cheng et al., Cancer Res., 54(21):5547-5551, 1994.
Christodoulides et al., Microbiology, 144(Pt 11):3027-3037, 1998.
Clifford et al., Br. J. Cancer, 88(1):63-73, 2003.
Cogliano et al., Lancet Oncol., 6(4):204, 2005.
Cox, J. Fam. Pract., Suppl:3-9, 2006.
Cummins et al., In: IRT: Nucleosides and nucleosides, La Jolla Calif., 72, 1996.
Davidson et al., J. Immunother., 21(5):389-398, 1998.
Davison et al., Meth Enzymol., 411:14-34, 2006.
de Villiers et al., Virology, 324(1):17-27, 2004.
Denli et al., Trends Biochem. Sci., 28:196, 2003.
Didenko, Biotechniques, 31(5):1106-16, 1118, 1120-1, 2001.
Doorbar et al., Nature, 352(6338):824-7, 1991.
Doorbar, Clin. Sci. (Lond), 110(5):525-41, 2006.
Draghici, In: Data Analysis Tools for DNA Microarrays, Chapman and Hall/CRC Publishing, 2003.
D'Souza et al., New Engl. J. Med., 356:1944-1956, 2007.
Emptage et al., Neuron, 29(1):197-208, 2001.
EP 266,032
EP 373 203
EP 785 280
EP 799 897
Esquela-Kerscher and Slack, Nat. Rev. Cancer, 6(4):259-269, 2006.
Fehrmann and Laimins, Methods Mol. Biol., 292:317-30, 2005.
Frattini et al., Proc. Natl. Acad. Sci. USA, 93(7):3062-7, 1996.
Froehler et al., Nucleic Acids Res., 14(13):5399-5407, 1986.
Gillison et al., J. Natl. Cancer Inst., 92(9):709-20, 2000.
Griffey et al., J. Mass Spectrom, 32(3):305-13, 1997.
Griffiths-Jones et al., Nucleic Acids Res., 34:D140-D144, 2006.
Hanibuchi et al., Int. J. Cancer, 78(4):480-485, 1998.
Harfe, Curr. Opin. Genet. Dev., 15(4):410-5, 2005.
He et al., Nature, 447(7148):1130-4, 2007.
Hellstrand et al., Acta Oncologica, 37(4):347-353, 1998.
Huber et al., Bioinformatics, 18(S1):S96-S104, 2002.
Hui and Hashimoto, Infection Immun., 66(11):5329-5336, 1998.
Hummel et al., J. Virol., 66(10):6070-80, 1992.
Hussussian et al., Nat. Genet., 8(1):15-21, 1994.
Itakura and Riggs, Science, 209:1401-1405, 1980.
Jemal et al., CA Cancer J. Clin., 57:43-66, 2007.
Jopling et al., Science, 309(5740):1577-81, 2005.
Kamb et al., Nat. Genet., 8(1):23-26, 1994.
Kamb et al., Science, 2674:436-440, 1994.
Klostermeier and Millar, Biopolymers, 61(3):159-79, 2001-2002.
Lambert, J. Virol., 65(7):3417-20, 1991.
Lecellier et al., Science, 308(5721):557-60, 2005.
Lee et al., Biochim. Biophys. Acta, 1582:175-177, 2002.
Longworth and Laimins, Microbiol. Mol. Biol. Rev., 68(2):362-72, 2004.
Marsters et al., Recent Prog. Horm. Res., 54:225-234, 1999.
McLaughlin-Drubin et al., Virology, 312(1):1-7, 2003.
Meyers et al., J. Virol., 71(10):7381-6, 1997.
Meyers et al., Science, 257(5072):971-3, 1992.
Meyers, In: Methods in Cell Science, 18:201-210, Kluwer Academic Publishers, Netherlands, 1996.
Mori et al., Cancer Res., 54(13):3396-3397, 1994.
Nobori et al., Nature, 368(6473):753-756, 1994.
Okamoto et al., Proc. Natl. Acad. Sci. USA, 91(23):11045-11049, 1994.
Olsen et al., Dev. Biol., 216:671, 1999.
Orlow et al., Cancer Res, 54(11):2848-2851, 1994.
Orlow et al., Int. J. Oncol., 15(1):17-24, 1994.
PCT Appln. WO 0138580
PCT Appln. WO 0168255
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100012
PCT Appln. WO 03100448A1
PCT Appln. WO 04020085
PCT Appln. WO 04027093
PCT Appln. WO 09923256
PCT Appln. WO 09936760
PCT Appln. WO 93/17126
PCT Appln. WO 95/11995
PCT Appln. WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 97/43450
PCT Appln. WO 99/35505
Pietras et al., Oncogene, 17(17):2235-2249, 1998.
Qin et al., Proc. Natl. Acad. Sci. USA, 95(24):14411-14416, 1998.
Rader et al., Oncogene, 5(4):571-6, 1990.
Remington's Pharmaceutical Sciences” 15^{th} Ed., 1035-1038 and 1570-1580, 1990.
Rose et al., Transplantation, 82(4):570-3, 2006.
Sambrook et al., In: DNA microaarays: a Molecular Cloning Manual, Cold Spring Harbor Laboratory Press, Cold Spring Harbor, N.Y., 2003.
Sambrook et al., In: Molecular cloning: a Laboratory Manual, 2^{nd} Ed., Cold Spring Harbor Laboratory Press, Cold Spring Harbor, N.Y., 1989.
Sambrook et al. In: Molecular cloning: a Laboratory Manual, 3^{rd} Ed., Cold Spring Harbor Laboratory Press, Cold Spring Harbor, N.Y., 2001.
Scaria et al., Cell Microbiol., (12):2784-94 2007.
Scaria et al., Retrovirology, 3:68, 2006.
Seggerson et al., Dev. Biol., 243:215, 2002.
Serrano et al., Nature, 366:704-707, 1993.
Serrano et al., Science, 267(5195):249-252, 1995.
Sevignani et al., Mamm. Genome, 17(3):189-202, 2006.
Shingara et al., RNA, 11:1461-1470, 2005.
Smith et al., Int. J. Cancer, 121(3):621-32, 2007.
Stubenrauch and Laimins, Semin. Cancer Biol., 9(6):379-86, 1999.
U.K. Patent 8 803 000
Walboomers et al., J. Pathol., 189(1):12-9, 1999.
Wiemer, Eur. J. Cancer, 43(10):1529-44, 2007.
Wilson and Laimins, Methods Mol. Med., 119:157-69, 2005.
Zhang et al., Dev. Biol., 302(1):1-12, 2007.
zur Hausen, Nat. Rev. Cancer, 2(5):342-50, 2002.

1. A method of modulating Human Papillomavirus (HPV) infection of a cell comprising administering to the cell an isolated
(a) nucleic acid inhibitor of hsa-miR-199a, hsa-miR-199a-AS, hsa-miR-214, hsa-miR-145, hsa-miR-143, hsa-miR-151, hsa-miR-30a_{—}5p, hsa-miR-130a, hsa-miR-197, hsa-miR-181b, hsa-miR-130b and/or hsa-miR-203; or
(b) nucleic acid having a hsa-miR-503, hsa-miR-194, hsa-miR-491, hsa-miR-224, hsa-miR-96, hsa-miR-132, hsa-miR-30e-5p, hsa miR-200a, and/or ambi-miR-7027 activity;
in an amount sufficient to modulate HPV infection.

2. The method of claim 1, wherein a hsa-miR-503 is administered.

3. The method of claim 1, wherein an inhibitor of hsa-miR-199a, hsa-miR-199a-AS, hsa-miR-214, hsa-miR-145, hsa-miR-143, hsa-miR-151, hsa-miR-30a_{—}5p is administered.

4. (canceled)

5. (canceled)

6. The method of claim 1, wherein the cell is in a subject having, suspected of having, or at risk of developing a HPV infection.

7. The method of claim 6, wherein the HPV infection is associated with a precancerous or cancerous condition.

8. The method of claim 7, wherein the precancerous condition is cervical intraepithelial neoplasia (CIN).

9. The method of claim 1, wherein the cell is a cancer cell.

10. The method of claim 1 wherein the cell is a keratinocyte.

11. The method of claim 1 wherein said HPV infection is reduced.

12. The method of claim 1 wherein said HPV infection is eliminated

13. The method of claim 1, wherein the isolated therapeutic nucleic acid is a recombinant nucleic acid.

14-18. (canceled)

19. The method of claim 1, wherein the therapeutic nucleic acid is a synthetic nucleic acid.

20. (canceled)

21. (canceled)

22. The method of claim 1, wherein the nucleic acid is comprised in a pharmaceutical formulation.

23. The method of claim 22, wherein the pharmaceutical formulation is a lipid composition, a nanoparticle composition, or a biocompatible and biodegradable composition.

24. (canceled)

25. (canceled)

26. The method of claim 1, further comprising administering 2, 3, 4, 5, 6, or more therapeutic nucleic acids.

27. The method claim 26, wherein the miRNAs are comprised in a single composition.

28. The method of claim 1, wherein viability of the cell is reduced, proliferation of the cell is reduced, metastasis of the cell is reduced, or the cell's sensitivity to therapy is increased.

29. (canceled)

30. (canceled)

31. A method of selecting a miRNA to be administered to a subject suspected of having, or having a propensity for developing a HPV infection comprising:
(a) determining an expression profile of one or more miRNA selected from ambi-miR-7027, hsa-miR-30a_{—}5p, hsa-miR-30e_{—}5p, hsa-miR-96, hsa-miR-130a, hsa-miR-130b, hsa-miR-132, hsa-miR-143, hsa-miR-145, hsa-miR-151, hsa-miR-181b, hsa-miR-194, hsa-miR-197, hsa-miR-199a, hsa-miR-199a-AS, hsa-miR-200a, hsa-miR-203, hsa-miR-214, hsa-miR-224, hsa-miR-491, and/or hsa-miR-503;
(b) assessing the sensitivity of the subject to a therapeutic nucleic acid based on the expression profile; and
(c) selecting one or more therapeutic nucleic acid for administration to the patient based on the assessed sensitivity.

32-34. (canceled)

35. A method of detecting HPV infection in a biological sample comprising evaluating expression levels of hsa-mir-199a, hsa-miR-199a-AS, hsa-miR-214, hsa-miR-145, hsa-miR-143, hsa-miR-151, hsa-miR-503, hsa-miR-30a_{—}5p, hsa-miR-200a, or combinations thereof.

36. The method of claim 35, wherein the biological sample is from a mucous membrane, skin, cervix, anus, rectum, penis, vulva, vagina, oropharynx, nasopharynx, trachea, esophagus, or epiglottis.

37-41. (canceled)
